(19) United States
(12) Patent Application Publication
(10) Pub. No.: US 2024/0237513 A1
(43) Pub. Date: Jul. 11, 2024

(54) COMPOUND, MATERIAL FOR ORGANIC ELECTROLUMINESCENT ELEMENT, ORGANIC ELECTROLUMINESCENT ELEMENT, AND ELECTRONIC APPLIANCE

(71) Applicant: IDEMITSU KOSAN CO., LTD., Tokyo (JP)

(72) Inventors: Tasuku HAKETA, Chiyoda-ku, Tokyo (JP); Kiyoshi IKEDA, Chiyoda-ku, Tokyo (JP); Shota TANAKA, Chiyoda-ku, Tokyo (JP); Yusuke TAKAHASHI, Chiyoda-ku, Tokyo (JP); Takuto FUKAMI, Chiyoda-ku, Tokyo (JP); Tsukasa SAWATO, Chiyoda-ku, Tokyo (JP)

(73) Assignee: IDEMITSU KOSAN CO., LTD., Tokyo (JP)

(21) Appl. No.: 18/287,222

(22) PCT Filed: Apr. 28, 2022
(86) PCT No.: PCT/JP2022/019218

§ 371 Date: Oct. 17, 2023

(30) Foreign Application Priority Data
Apr. 28, 2021 (JP) 2021-076693

(51) Int. Cl. H10K 85/60; C07D 307/91; C09K 11/06; H10K 50/15

(52) U.S. Cl. CPC H10K 85/636; C07D 307/91; C09K 11/06; H10K 85/633; C09K 2211/1018; H10K 50/156; H10K 85/626; H10K 85/6574

(57) ABSTRACT

The present invention is to provide a compound and a material for organic electroluminescent devices that further improve the performance of an organic EL device, an organic electroluminescent device having further improved device performance, and an electronic appliance including such an organic electroluminescent device. Provided is a compound represented by the following formula (1) (the signs in the formula are as defined in the description), a material for organic electroluminescent devices, the material containing the compound, an organic electroluminescent device containing the compound, and an electronic appliance including such an organic electroluminescent device.

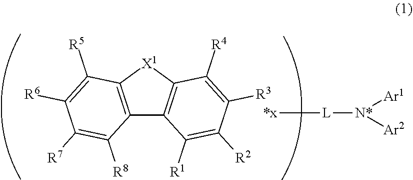

### TECHNICAL FIELD

The present invention relates to a compound, a material for organic electroluminescent devices, an organic electroluminescent device, and an electronic appliance including the organic electroluminescent device.

### BACKGROUND ART

In general, an organic electroluminescent device (hereinafter sometimes referred to as an “organic EL device”) is composed of an anode, a cathode, and an organic layer interposed between the anode and the cathode. When a voltage is applied between the two electrodes, electrons from the cathode side and holes from the anode side are injected into a light emitting region, and the injected electrons and holes are recombined in the light emitting region to generate an excited state, which then returns to the ground state to emit light. Accordingly, development of a material that efficiently transports electrons or holes into the light emitting region to promote recombination of electrons and holes is important for providing a high performance organic EL device.

PTLs 1 to 3 disclose compounds to be used as a material for organic electroluminescent devices.

### CITATION LIST

### Patent Literature

PTL 1: US 2018/226585 A1
PTL 2: JP 2014-527021 A
PTL 3: JP 2018-524289 A

### SUMMARY OF INVENTION

### Technical Problem

Although many compounds for organic EL devices have conventionally been reported, a compound that further increases the performance of an organic EL device remains to be desired.

The present invention has been made for solving the above problem, and an object of the present invention is to provide a compound and a material for organic electroluminescent devices that further improve the performance of an organic EL device, an organic EL device having further improved device performance, and an electronic appliance including such an organic EL device.

As a result of intensive and extensive studies on the performance of organic EL devices containing novel compounds, the present inventors have found that an organic EL device containing a compound represented by the following formula (1) shows further improved performance.

In an aspect, the present invention provides a compound represented by the following formula (1):

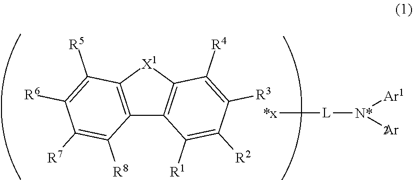

wherein
N* is a central nitrogen atom;
X¹ is an oxygen atom or a sulfur atom;
one selected from R¹ to R³ and R⁶ to R⁸ is a single bond bonded to *x;
R⁴ and R⁵, and R¹ to R³ and R⁶ to R⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R⁴ and R⁵, and R¹ to R³ and R⁶ to R⁸ that are not a single bond each are not bonded to each other and form no ring;
L is an unsubstituted phenylene group;
Ar¹ and Ar² are each independently represented by any of the following formulae (1-a) to (1-f);

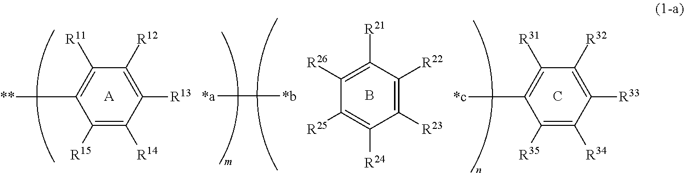

wherein
** represents a binding position to the central nitrogen atom N*;
one selected from R¹¹ to R¹⁵ is a single bond bonded to *a, one selected from R²¹ to R²⁶ is a single bond bonded to *b, and another one selected from R²¹ to R²⁶ is a single bond bonded to *c;
R¹¹ to R¹⁵ that are not a single bond are each independently a hydrogen atom, an unsubstituted alkyl group having 1 to 6 carbon atoms, or an unsubstituted aryl group having 6 to 12 ring carbon atoms;
R²¹ to R²⁶ that are not a single bond are each independently a hydrogen atom, an unsubstituted alkyl group having 1 to 6 carbon atoms, or an unsubstituted aryl group having 6 to 12 ring carbon atoms;
R³¹ to R³⁵ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R¹¹ to R¹⁵ that are not a single bond and of R²¹ to R²⁶ that are not a single bond each are bonded to each other to form a substituted or unsubstituted monocyclic ring having 3 or more and 6 or less ring atoms or each are not bonded to each other and form no ring;
one or more combinations of combinations each including adjacent two or more of R³¹ to R³⁵ that are not a single bond each are not bonded to each other and form no ring;
m is 0 or 1,
n is 0 or 1, and
m≥n, provided that
when m and n is 0, *c is bonded to the central nitrogen atom N*, and
when m is 1 and n is 0, one selected from R¹¹ to R¹⁵ is a single bond bonded to *c;

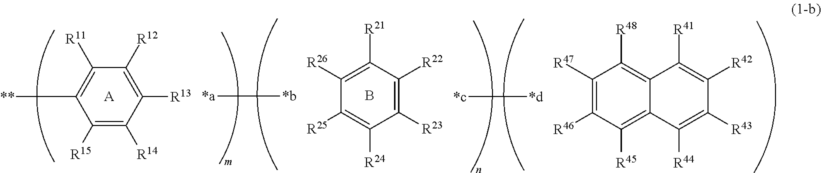

wherein
**, *a, *b, *c, R¹¹ to R¹⁵, R²¹ to R²⁶, m, and n are as defined in the formula (1-a);
one selected from R⁴¹ to R⁴⁸ is a single bond bonded to *d, and R⁴¹ to R⁴⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
when R⁴¹ to R⁴⁸ that are not a single bond are each a hydrogen atom, all the hydrogen atoms are each a protium atom;
one or more combinations of combinations each including adjacent two or more of R⁴¹ to R⁴⁸ that are not a single bond each are not bonded to each other and form no ring;

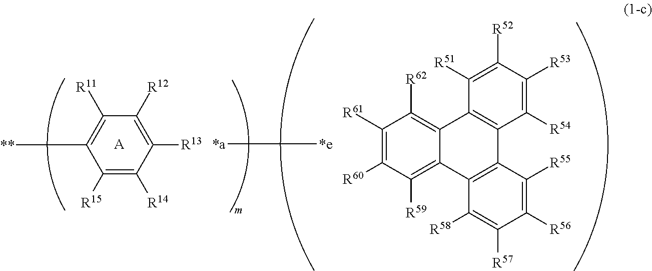

wherein
**, *a, R¹¹ to R¹⁵, and m are as defined in the formula (1-a);
one selected from R⁵¹ to R⁶² is a single bond bonded to *e, and R⁵¹ to R⁶² that are not a single bond are each independently a hydrogen atom or a substituted or unsubstituted alkyl group having 1 to 6 carbon atoms;
one or more combinations of combinations each including adjacent two or more of R⁵¹ to R⁶² that are not a single bond each are not bonded to each other and form no ring;
provided that when m is 0, *a is bonded to the central nitrogen atom N*;

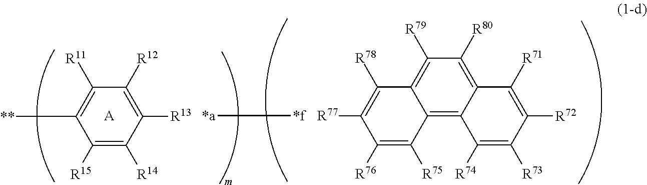

wherein
**, *a, R¹¹ to R¹⁵, and m are as defined in the formula (1-a);
one selected from R⁷¹ to R⁷⁸ is a single bond bonded to *f, and R⁷⁹ and R⁸⁰, and R⁷¹ to R⁷⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R⁷⁹ and R⁸⁰, and R⁷¹ to R⁷⁸ that are not a single bond each are not bonded to each other and form no ring;
provided that when m is 0, *a is bonded to the central nitrogen atom N*;

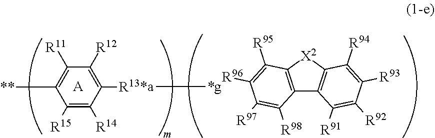

wherein
**, *a, R¹¹ to R¹⁵, and m are as defined in the formula (1-a);
X² is an oxygen atom, a sulfur atom, or CR^{A}R^{B};
R^{A} and R^{B} are each independently a substituted or unsubstituted alkyl group having 1 to 6 carbon atoms or a substituted or unsubstituted aryl group having 6 to 12 ring carbon atoms;
R^{A} and R^{B} are bonded to each other to form a substituted or unsubstituted monocyclic ring, are bonded to each other to form a substituted or unsubstituted condensed ring, or are not bonded to each other and form no ring;
one selected from R⁹¹ to R⁹⁸ is a single bond bonded to *g, and R⁹¹ to R⁹⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R⁹¹ to R⁹⁸ that are not a single bond each are bonded to each other to form a substituted or unsubstituted monocyclic ring having 3 or more and 6 or less ring atoms, or each are not bonded to each other and form no ring;
provided that when m is 0 and X² is an oxygen atom, one selected from R⁹² to R⁹⁷ is a single bond bonded to *g, and *a is bonded to the central nitrogen atom N*.

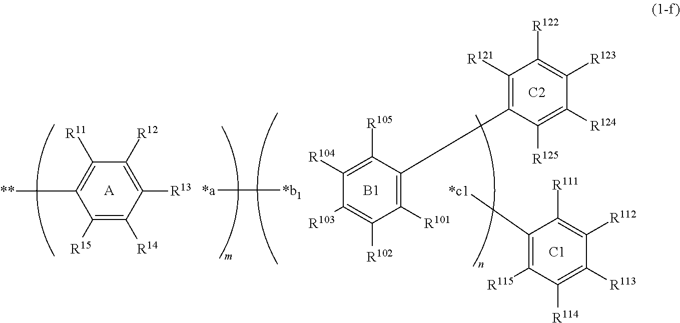

wherein
**, *a, R¹¹ to R¹⁵, and m are as defined in the formula (1-a);
one selected from R¹⁰¹ to R¹⁰⁵ is a single bond bonded to *b1, and another one selected from R¹⁰¹ to R¹⁰⁵ is a single bond bonded to *c1;
R¹¹¹ to R¹¹⁵ and R¹²¹ to R¹²⁵ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R¹⁰¹ to R¹⁰⁵ that are not a single bond are each independently a hydrogen atom, an unsubstituted alkyl group having 1 to 6 carbon atoms, or an unsubstituted aryl group having 6 to 12 ring carbon atoms;
one or more combinations of combinations each including adjacent two or more of R¹⁰¹ to R¹⁰⁵ that are not a single bond each are not bonded to each other and form no ring;
one or more combinations of combinations each including adjacent two or more of R¹¹¹ to R¹¹⁵ and R¹²¹ to R¹²⁵ each are not bonded to each other and form no ring;
provided that when m is 0, *a is bonded to the central nitrogen atom N*; and wherein in the formula (1), at least one of hydrogen atoms contained in L, R¹¹ to R¹⁵ that are not a single bond, R²¹ to R²⁶ that are not a single bond, R⁵¹ to R⁶² that are not a single bond, R⁷¹ to R⁸⁰ that are not a single bond, R⁹¹ to R⁹⁸ that are not a single bond, R^{A} to R^{B}, and R¹⁰¹ to R¹⁰⁵ that are not a single bond is a deuterium atom.

In another aspect, the present invention provides a material for organic electroluminescent devices, the material containing the compound represented by the formula (1).

In still another aspect, the present invention provides an organic electroluminescent device including a cathode, an anode, and organic layers between the cathode and the anode, the organic layers including a light emitting layer, at least one layer of the organic layers containing the compound represented by the formula (1).

In yet another aspect, the present invention provides an electronic appliance including the organic electroluminescent device.

### Advantageous Effects of Invention

An organic EL device containing the compound represented by the formula (1) shows improved device performance.

### BRIEF DESCRIPTION OF DRAWINGS

FIG. 1 is a schematic illustration showing an example of the layer configuration of an organic EL device according to an aspect of the present invention.

FIG. 2 is a schematic illustration showing another example of the layer configuration of an organic EL device according to an aspect of the present invention.

FIG. 3 is a schematic illustration showing still another example of the layer configuration of an organic EL device according to an aspect of the present invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A)

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

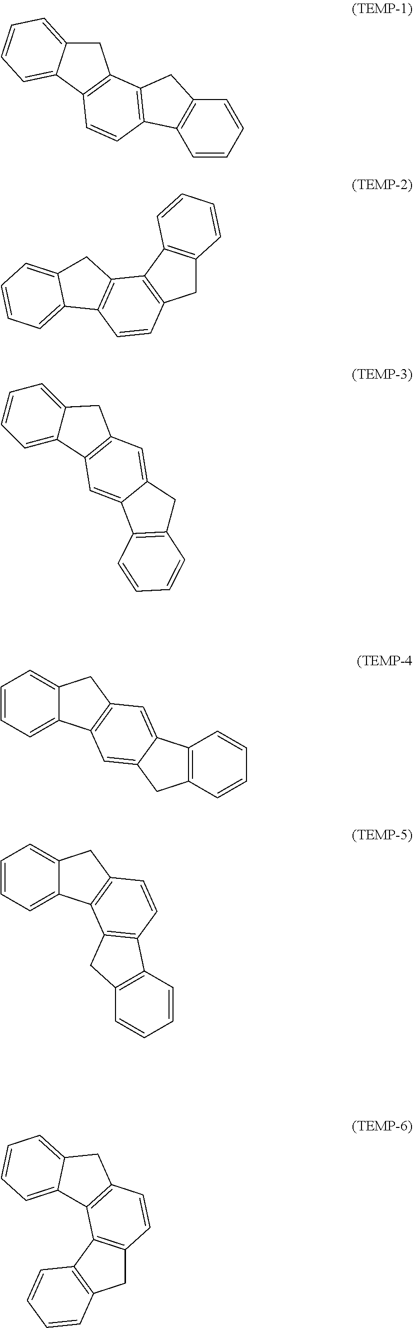

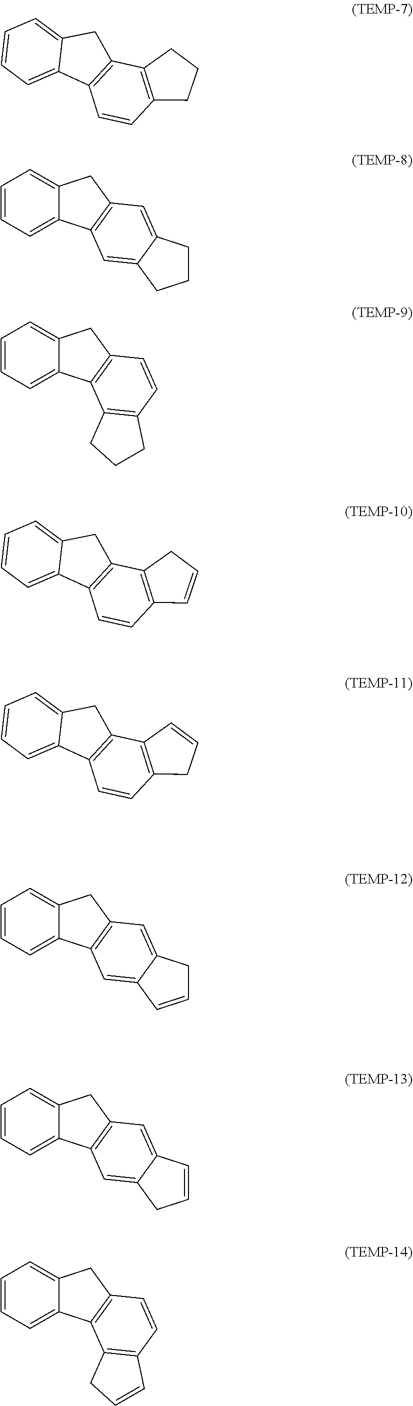

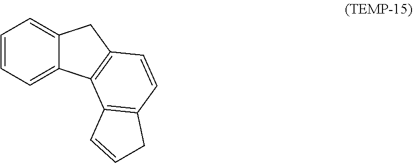

### Substituted Aryl Group (Set of Specific Examples G1B)

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

### Unsubstituted Heterocyclic Group Containing Nitrogen Atom (Set of Specific Examples G2A1)

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and a diazacarbazolyl group.

### Unsubstituted Heterocyclic Group Containing Oxygen Atom (Set of Specific Examples G2A2)

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing Sulfur Atom (Set of Specific Examples G2A3)

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

### Monovalent Heterocyclic Group Derived by Removing One Hydrogen Atom from Ring Structures Represented by General Formulae (TEMP-16) to (TEMP-33) (Set of Specific Examples G2A4)

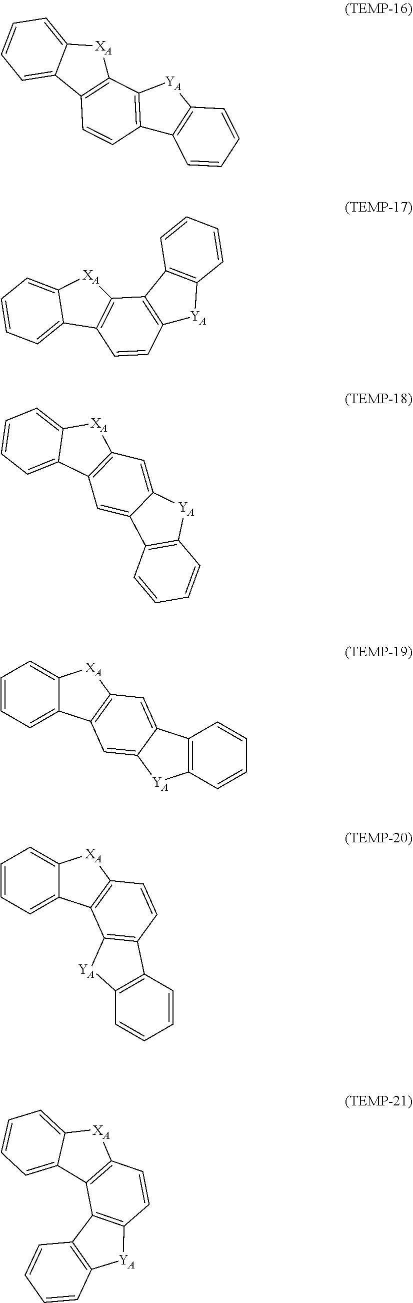

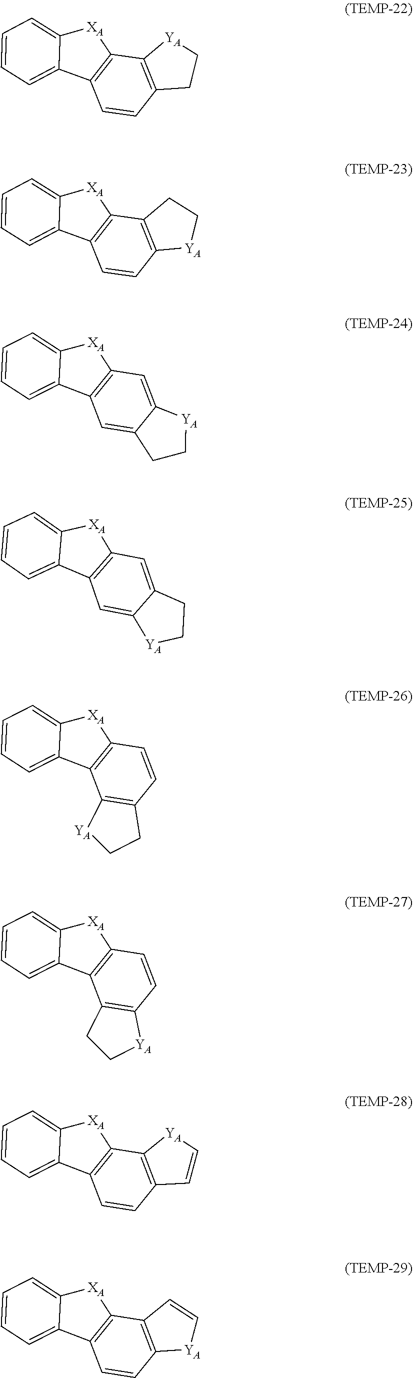

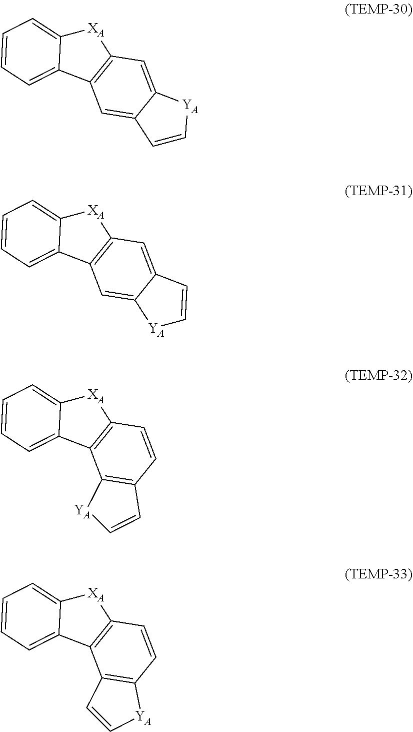

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

### Substituted Heterocyclic Group Containing Nitrogen Atom (Set of Specific Examples G211)

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and
a biphenylquinazolinyl group.

### Substituted Heterocyclic Group Containing Oxygen Atom (Set of Specific Examples G2B2)

a phenyldibenzofuranyl group,
a methyldibenzofuranyl group,
a t-butyldibenzofuranyl group, and
a monovalent residual group of spiro[9H-xanthene-9,9′-[9H]fluorene].

### Substituted Heterocyclic Group Containing Sulfur Atom (Set of Specific Examples G2B3)

a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

### Group Formed by Substituting One or More Hydrogen Atom of Monovalent Heterocyclic Group Derived from Ring Structures Represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A)

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B)

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A)

a vinyl group,
an allyl group,
a 1-butenyl group,
a 2-butenyl group, and
a 3-butenyl group.

### Substituted Alkenyl Group (Set of Specific Examples G4B)

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

### Unsubstituted Alkynyl Group (Set of Specific Examples G5A)

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A)

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Set of Specific Examples G6B)

a 4-methylcyclohexyl group.
   Group represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

Group represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Group represented by —S—(R₉₀₅) In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G1),
—N(G2)(G2),
—N(G1)(G2),
—N(G3)(G3), and
—N(G6)(G6).
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G1l) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(GT), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

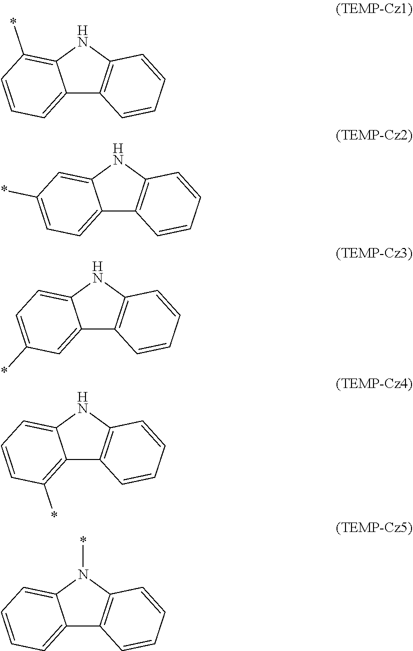

In the description herein, the (9-phenyl)carbazolyl group is specifically any one of the following groups unless otherwise indicated in the description.

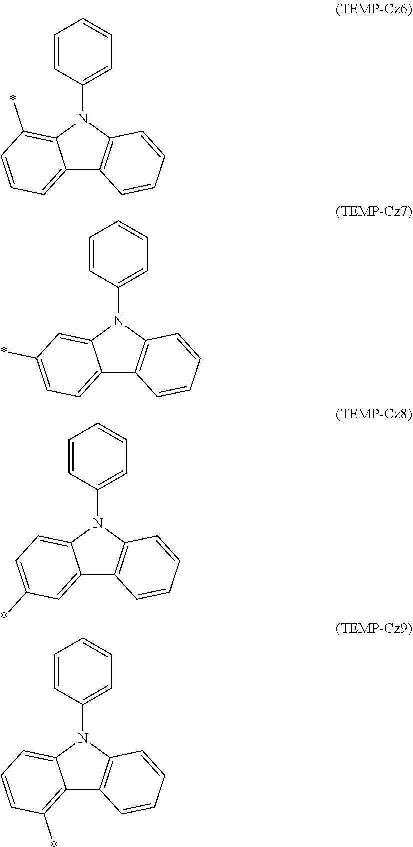

In the general formulae (TEMP-Czl) to (TEMP-Cz9), * represents a bonding site.

In the description herein, the dibenzofuranyl group and the dibenzothiophenyl group are specifically any one of the following groups unless otherwise indicated in the description.

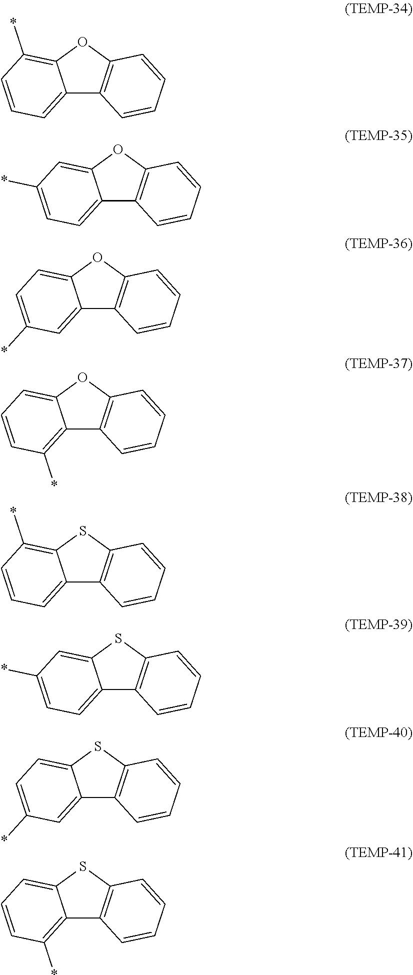

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

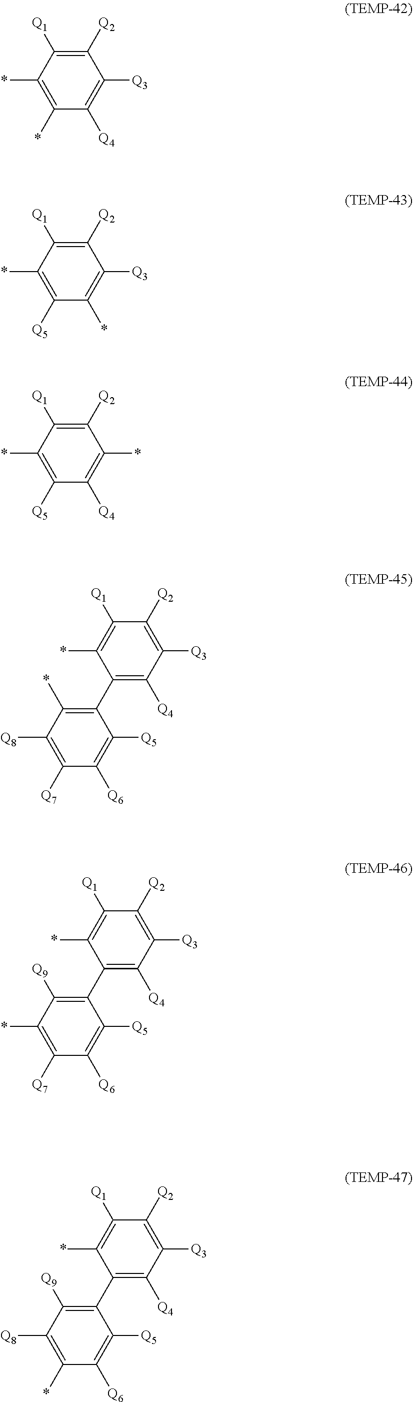

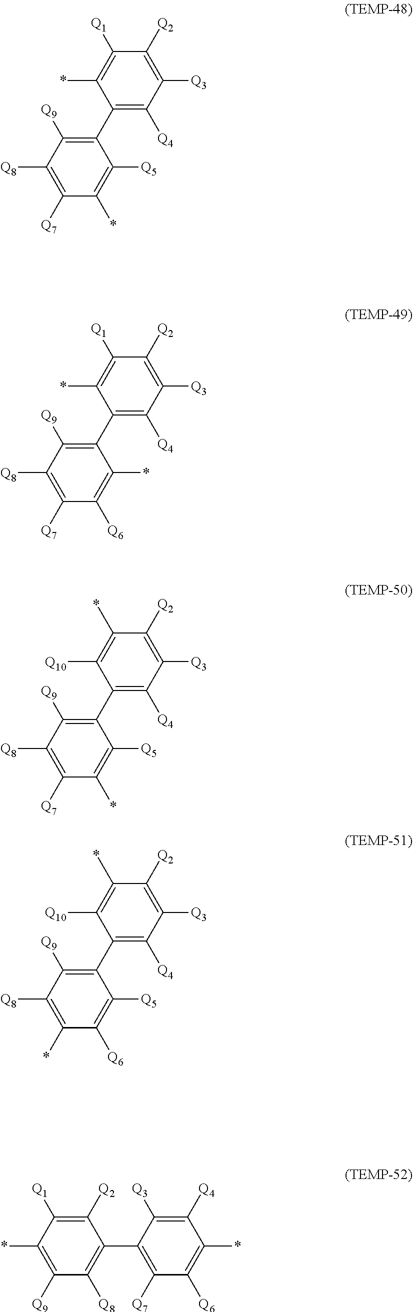

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

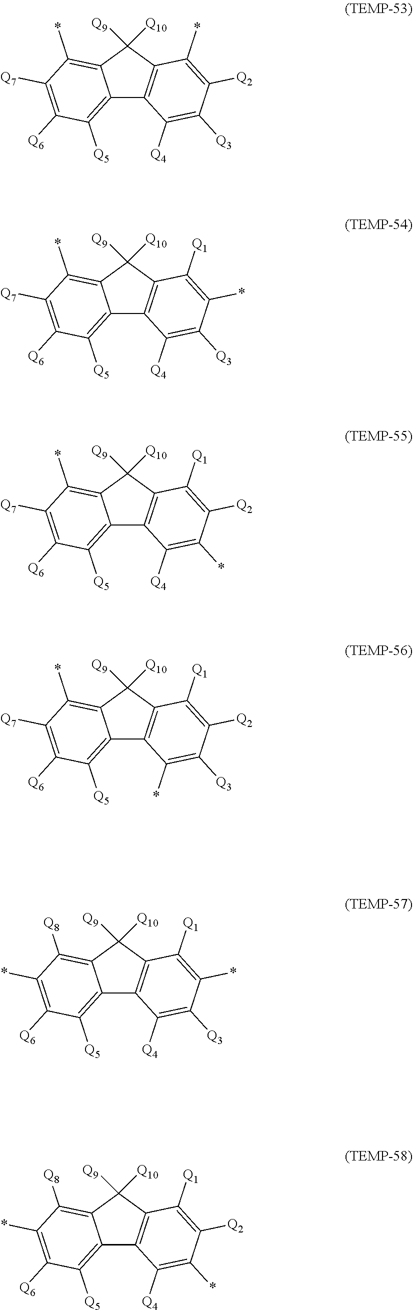

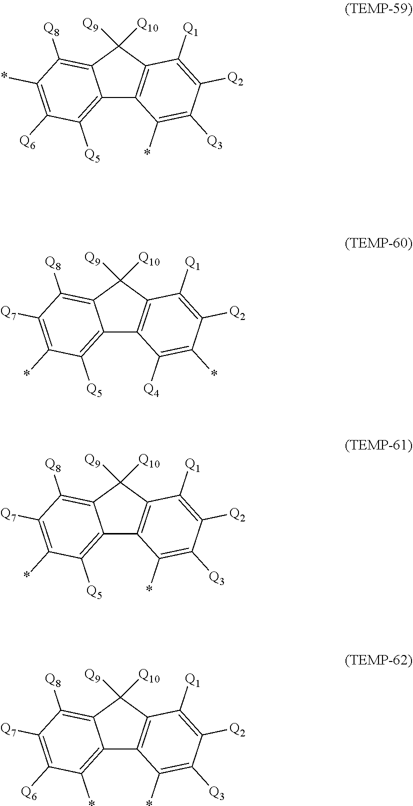

In the general formulae (TEMP-53) to (TEMP-6), Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

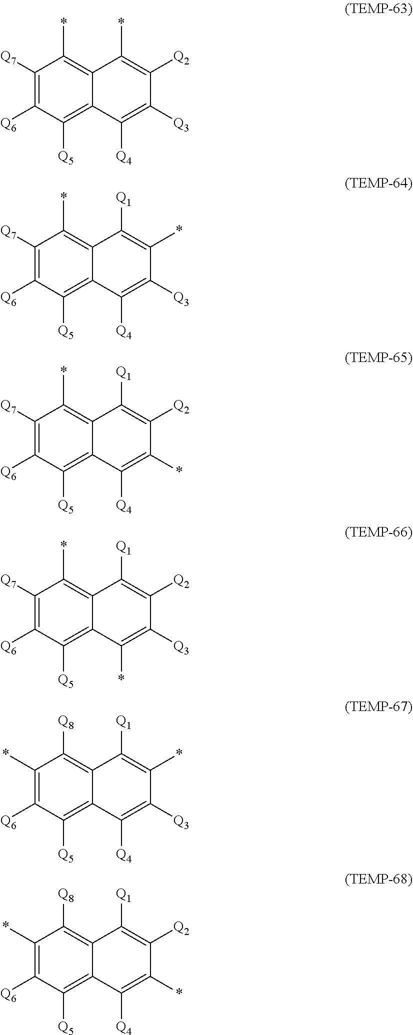

In the general formulae (TEMP-63) to (TEMP-68), Q₁ to Q₅ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

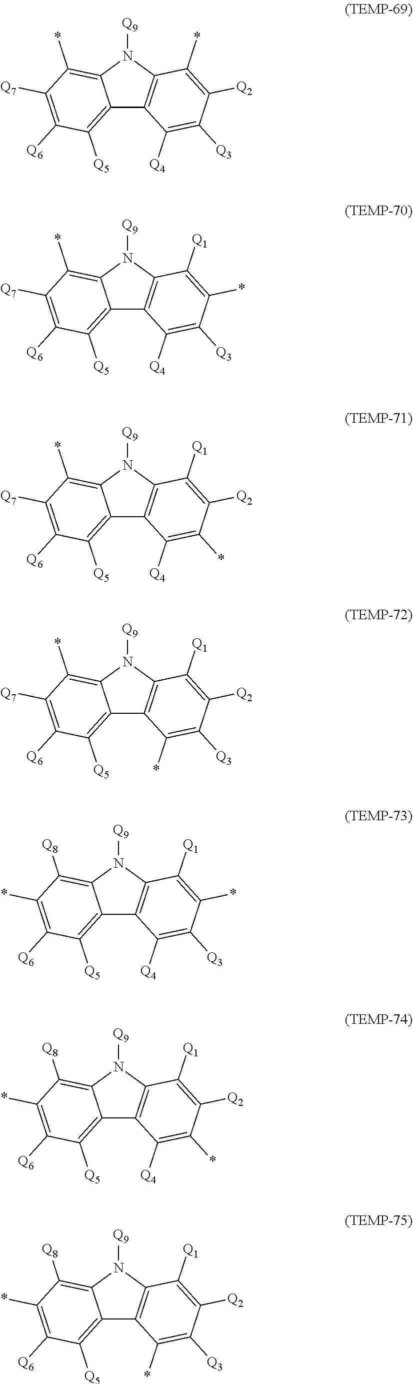

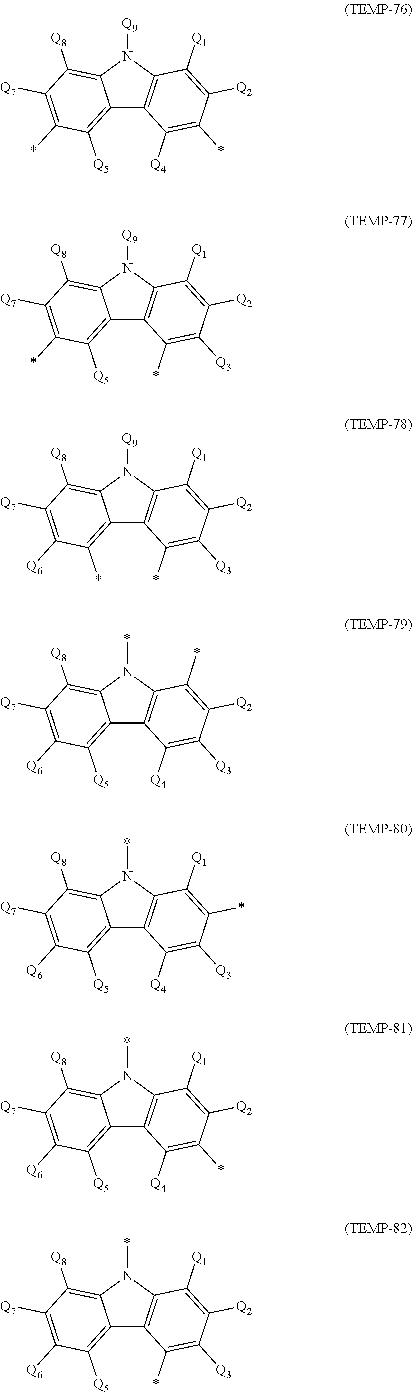

In the general formulae (TEMP-69) to (TEMP-82), Q₁ to Q₉ each independently represent a hydrogen atom or a substituent.

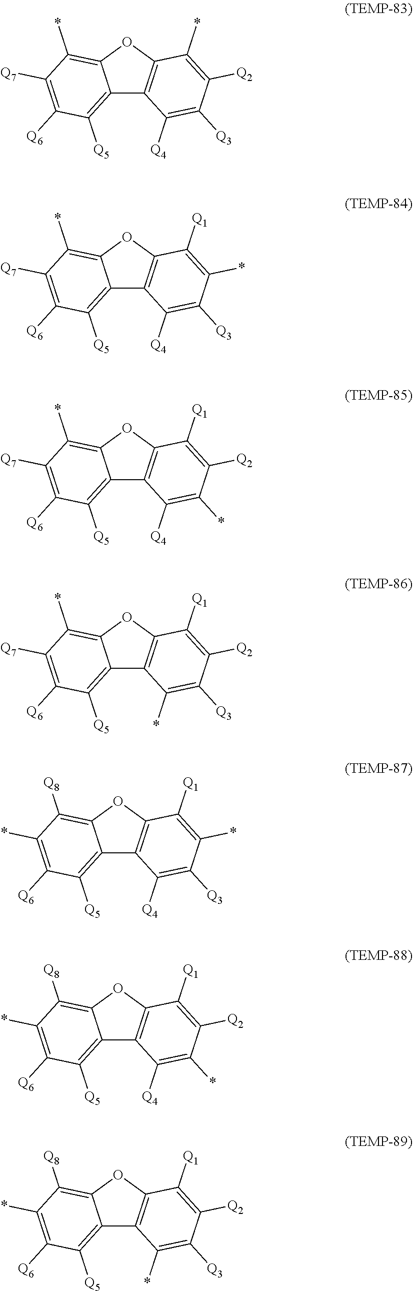

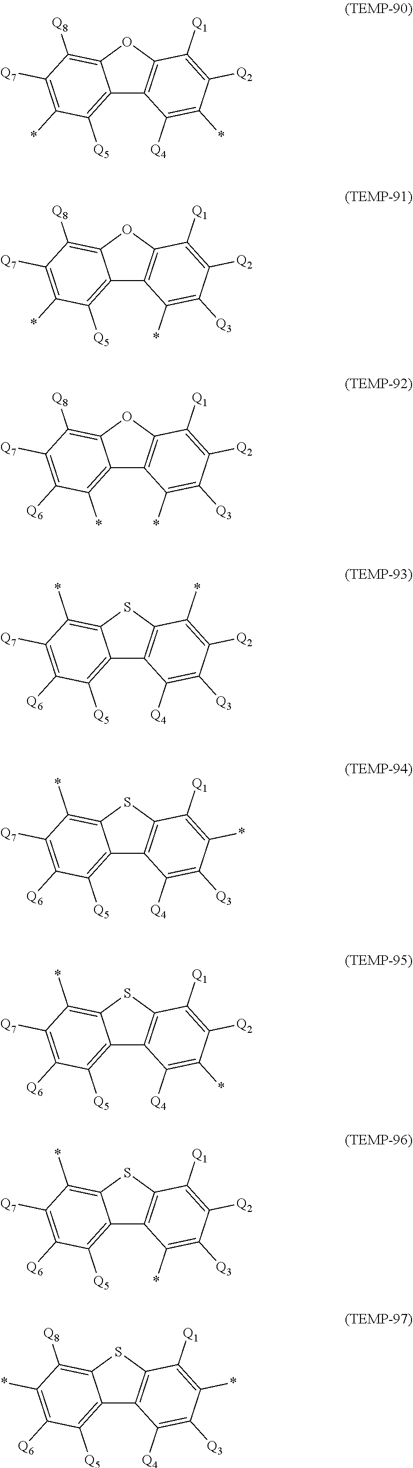

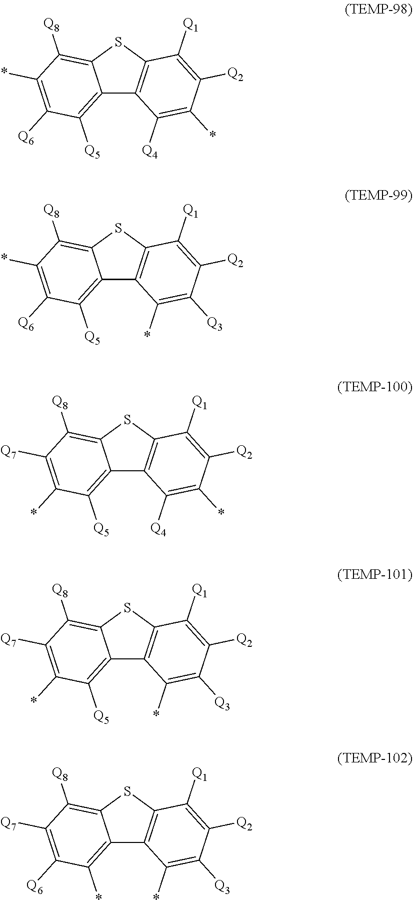

In the general formulae (TEMP-83) to (TEMP-102), Q₁ to Q₅ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

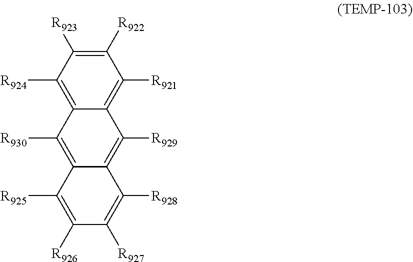

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

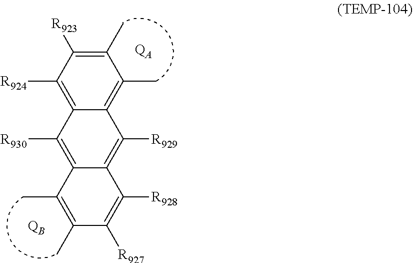

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Qc, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Qc share R₉₂₂.

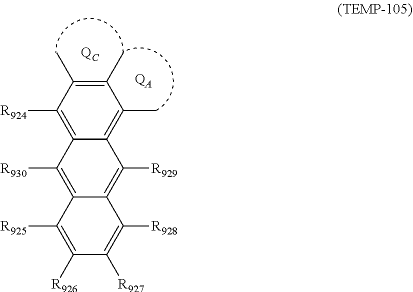

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Qc formed in the general formula (TEMP-105) each are a “condensed ring”. The ring Q_{A} and the ring Qc in the general formula (TEMP-105) form a condensed ring through condensation of the ring Q_{A} and the ring Qc. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or a non-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

The compound of the present invention will be described below.

The compound according to an aspect of the present invention is represented by the following formula (1),

The compounds of the present invention represented by the formula (1) and the formulae encompassed in the formula (1) which are described later are sometimes referred to simply as the “inventive compound (1)” or the “inventive compound”.

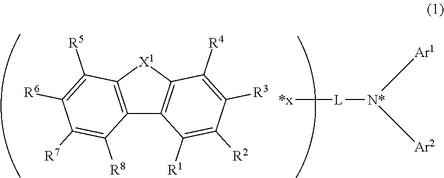

The signs in the formula (1) and the formulae encompassed in the formula (1) which are described later will be described below. Note that the same sign has the same meaning.

In the formula (1), N* is a central nitrogen atom.

In the formula (1), X¹ is an oxygen atom or a sulfur atom, and preferably an oxygen atom.

In the formula (1), one selected from R¹ to R³ and R⁶ to R⁸ is a single bond bonded to *x.

R⁴ and R⁵, and R¹ to R³ and R⁶ to R⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms, and preferably a hydrogen atom.

One or more combinations of combinations each including adjacent two or more of R⁴ and R⁵, and R¹ to R³ and R⁶ to R⁸ that are not a single bond each are not bonded to each other and form no ring.

The unsubstituted alkyl group represented by R⁴ and R⁵, and R¹ to R³ and R⁶ to R⁸ that are not a single bond is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R⁴ and R⁵, and R¹ to R³ and R⁶ to R⁸ that are not a single bond is preferably a phenyl group, a biphenyl group, or a naphthyl group, and more preferably a phenyl group.

The unsubstituted heterocyclic group represented by R⁴ and R⁵, and R¹ to R³ and R⁶ to R⁸ that are not a single bond is preferably a pyridyl group or a quinazolinyl group.

In the formula (1), L is an unsubstituted phenylene group.

The unsubstituted phenylene group may be any of a p-phenylene group, a m-phenylene group, and an o-phenylene group, preferably a p-phenylene group or a m-phenylene group, and more preferably a p-phenylene group.

All the hydrogen atoms directly bonded to the phenylene group represented by L are preferably a deuterium atom.

In the formula (1), Ar¹ and Ar² are each independently represented by any of the following formulae (1-a) to (1-f.

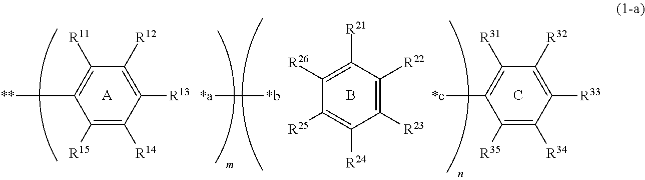

In the formula (1-a), ** represents a binding position to the central nitrogen atom N*.

In the formula (1-a), one selected from R¹¹ to R¹⁵ is a single bond bonded to *a, one selected from R²¹ to R²⁶ is a single bond bonded to *b, and another one selected from R²¹ to R²⁶ is a single bond bonded to *c.

R¹¹ to R¹⁵ that are not a single bond are each independently a hydrogen atom, an unsubstituted alkyl group having 1 to 6 carbon atoms, or an unsubstituted aryl group having 6 to 12 ring carbon atoms.

R²¹ to R²⁶ that are not a single bond are each independently a hydrogen atom, an unsubstituted alkyl group having 1 to 6 carbon atoms, or an unsubstituted aryl group having 6 to 12 ring carbon atoms.

The unsubstituted alkyl group represented by R¹¹ to R¹⁵ and R²¹ to R²⁶ is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R¹¹ to R¹⁵ and R²¹ to R²⁶ is preferably a phenyl group, a biphenyl group, or a naphthyl group, and more preferably a phenyl group.

In the formula (1-a), R³¹ to R³⁵ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms.

The unsubstituted alkyl group represented by R³¹ to R³⁵ is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R³¹ to R³⁵ is preferably a phenyl group, a biphenyl group, or a naphthyl group, and more preferably a phenyl group.

The unsubstituted heterocyclic group represented by R³¹ to R³⁵ is preferably a pyridyl group or a quinazolinyl group.

In the formula (1-a), one or more combinations of combinations each including adjacent two or more of R¹¹ to R¹⁵ that are not a single bond and of R²¹ to R²⁶ that are not a single bond each are bonded to each other to form a substituted or unsubstituted monocyclic ring having 3 or more and 6 or less ring atoms, or each are not bonded to each other and form no ring.

In the formula (1-a), one or more combinations of combinations each including adjacent two or more of R³¹ to R³⁵ that are not a single bond each are not bonded to each other and form no ring.

The unsubstituted monocyclic ring formed by R¹¹ to R¹⁵ that are not a single bond or R²¹ to R²⁶ that are not a single bond is, for example, a benzene ring or a cyclohexane ring, and preferably a benzene ring.

In the formula (1-a),
m is 0 or 1,
n is 0 or 1, and
m≥n, provided that
when m and n is 0, *c is bonded to the central nitrogen atom N*, and
when m is 1 and n is 0, one selected from R¹¹ to R¹⁵ is a single bond bonded to *c.

In the formula (1-a), m and n may both be 0, m and n may respectively be 1 and 0, or m and n may both be 1, but it is preferred that m is 1, and it is more preferred that m and n are respectively 1 and 0.
when m is 1, it is preferred that all the hydrogen atoms represented by R¹¹ to R¹⁵ that are not a single bond are each a deuterium atom.

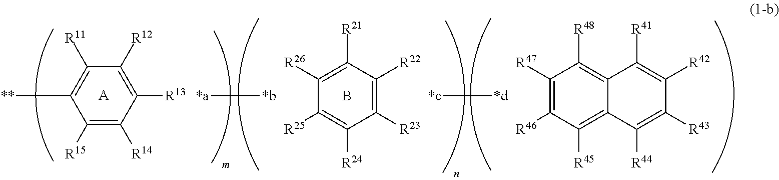

In the formula (1-b), **, *a, *b, *c, R¹¹ to R¹⁵, R²¹ to R²⁶, m, and n are as defined in the formula (1-a). In the formula (1-b), preferred groups represented by R¹¹ to R¹⁵ and R²¹ to R²⁶ are also the same as described for the formula (1-a).

In the formula (1-b), one selected from R⁴¹ to R⁴⁸ is a single bond bonded to *d, and R⁴¹ to R⁴⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms.

When R⁴¹ to R⁴⁸ that are not a single bond are each a hydrogen atom, all the hydrogen atoms are each a protium atom.

One or more combinations of combinations each including adjacent two or more of R⁴¹ to R⁴⁸ that are not a single bond each are not bonded to each other and form no ring.

The unsubstituted alkyl group represented by R⁴¹ to R⁴⁸ is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R⁴¹ to R⁴⁸ is preferably a phenyl group, a biphenyl group, or a naphthyl group, and more preferably a phenyl group.

The unsubstituted heterocyclic group represented by R⁴¹ to R⁴⁸ is preferably a pyridyl group or a quinazolinyl group.

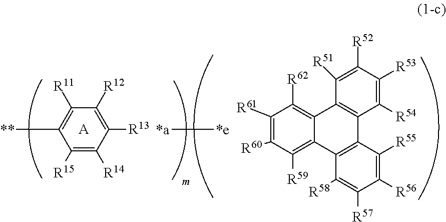

In the formula (1-c), **, *a, R¹¹ to R¹⁵, and m are as defined in the formula (1-a). In the formula (1-c), preferred groups represented by R¹¹ to R¹⁵ are also the same as described for the formula (1-a).

In the formula (1-c), one selected from R⁵¹ to R⁶² is a single bond bonded to *e. R⁵¹ to R⁶² that are not a single bond are each independently a hydrogen atom or a substituted or unsubstituted alkyl group having 1 to 6 carbon atoms.

One or more combinations of combinations each including adjacent two or more of R⁵¹ to R⁶² that are not a single bond each are not bonded to each other and form no ring.

Note that, when m is 0, *a is bonded to the central nitrogen atom N*.

The unsubstituted alkyl group represented by R⁵¹ to R⁶² is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

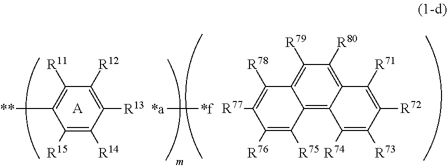

In the formula (1-d), **, *a, R¹¹ to R¹⁵, and m are as defined in the formula (1-a). In the formula (1-d), preferred groups represented by R¹¹ to R¹⁵ are also the same as described for the formula (1-a).

In the formula (1-d), one selected from R⁷¹ to R⁷⁸ is a single bond bonded to *f. R⁷⁹ and R⁸⁰, and R⁷¹ to R⁷⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms.

One or more combinations of combinations each including adjacent two or more of R⁷⁹ and R⁸⁰, and R⁷¹ to R⁷⁸ that are not a single bond each are not bonded to each other and form no ring.

Note that, when m is 0, *a is bonded to the central nitrogen atom N*.

The unsubstituted alkyl group represented by R⁷⁹ and R⁸⁰, and R⁷¹ to R⁷⁸ that are not a single bond is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R⁷⁹ and R⁸⁰, and R⁷¹ to R⁷⁸ that are not a single bond is preferably a phenyl group, a biphenyl group, or a naphthyl group, and more preferably a phenyl group.

The unsubstituted heterocyclic group represented by R⁷⁹ and R⁸⁰, and R⁷¹ to R⁷⁸ that are not a single bond is preferably a pyridyl group or a quinazolinyl group.

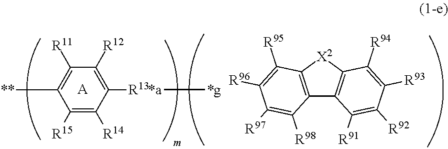

In the formula (1-e), **, *a, R¹¹ to R¹⁵, and m are as defined in the formula (1-a). In the formula (1-e), preferred groups represented by R¹¹ to R¹⁵ are also the same as described for the formula (1-a).

In the formula (1-e), X² is an oxygen atom, a sulfur atom, or CR^{A}R^{B}.

R^{A} and R^{B} are each independently a substituted or unsubstituted alkyl group having 1 to 6 carbon atoms or a substituted or unsubstituted aryl group having 6 to 12 ring carbon atoms.

The unsubstituted alkyl group represented by R^{A} and R^{B} is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R^{A} and R^{B} is preferably a phenyl group, a biphenyl group, or a naphthyl group, and more preferably a phenyl group. R^{A} and R^{B} are bonded to each other to form a substituted or unsubstituted monocyclic ring, are bonded to each other to form a substituted or unsubstituted condensed ring, or are not bonded to each other and form no ring.

The unsubstituted monocyclic ring formed by R^{A} and R^{B} is, for example, a benzene ring, a cyclopentane ring, or a cyclohexane ring.

The unsubstituted condensed ring formed by R^{A} and R^{B} is, for example, a naphthalene ring or an anthracene ring.

When R^{A} and R^{B} are bonded to each other to form an unsubstituted monocyclic ring or an unsubstituted condensed ring, R^{A} and R^{B}, together with a fluorene skeleton that is bonded thereto, may form a ring, such as a spirobifluorene skeleton or a spiro[9H-fluorene-9,1′-cyclopentane] skeleton.

In the formula (1-e), one selected from R⁹¹ to R⁹⁸ is a single bond bonded to *g. R⁹¹ to R⁹⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms.

The unsubstituted alkyl group represented by R⁹¹ to R⁹⁸ that are not a single bond is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R⁹¹ to R⁹⁸ that are not a single bond is preferably a phenyl group, a biphenyl group, or a naphthyl group, and more preferably a phenyl group.

The unsubstituted heterocyclic group represented by R⁹¹ to R⁹⁸ that are not a single bond is preferably a pyridyl group or a quinazolinyl group.

In the formula (1-e), one or more combinations of combinations each including adjacent two or more of R⁹¹ to R⁹⁸ that are not a single bond each are bonded to each other to form a substituted or unsubstituted monocyclic ring having 3 or more and 6 or less ring atoms, or each are not bonded to each other and form no ring.

Note that, when m is 0 and X² is an oxygen atom, one selected from R⁹² to R⁹⁷ is a single bond bonded to *g, and *a is bonded to the central nitrogen atom N*.

The unsubstituted monocyclic ring having 3 or more and 6 or less ring atoms formed by R⁹¹ to R⁹⁸ that are not a single bond is, for example, a benzene ring or a cyclohexane ring, and preferably a benzene ring.

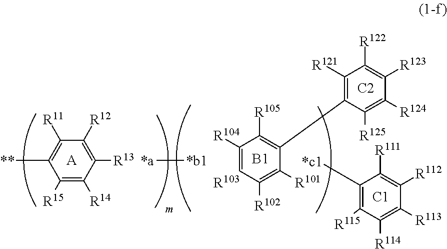

In the formula (1-f), **, *a, R¹¹ to R¹⁵, and m are as defined in the formula (1-a). In the formula (1-f), preferred groups represented by R¹¹ to R¹⁵ are also the same as described for the formula (1-a).

In the formula (1-f), one selected from R¹⁰¹ to R¹⁰⁵ is a single bond bonded to *b1 and another one selected from R¹⁰¹ to R¹⁰⁵ is a single bond bonded to *c1.

In the formula (1-f), R¹¹¹ to R¹¹⁵ and R¹²¹ to R¹²⁵ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms.

In the formula (1-f), R¹⁰¹ to R¹⁰⁵ that are not a single bond are each independently a hydrogen atom, an unsubstituted alkyl group having 1 to 6 carbon atoms, or an unsubstituted aryl group having 6 to 12 ring carbon atoms.

One or more combinations of combinations each including adjacent two or more of R¹⁰¹ to R¹⁰⁵ that are not a single bond each are not bonded to each other and form no ring.

In the formula (1-f), one or more combinations of combinations each including adjacent two or more of R¹¹¹ to R¹¹⁵ and R¹²¹ to R¹²⁵ each are not bonded to each other and form no ring.

In the formula (1-f), when m is 0, *a is bonded to the central nitrogen atom N*.

The unsubstituted alkyl group represented by R¹⁰¹ to R¹⁰⁵ that are not a single bond, R¹¹¹ to R¹¹⁵, and R¹²¹ to R¹²⁵ is preferably a methyl group, an ethyl group, an n-propyl group, an isopropyl group, an n-butyl group, an isobutyl group, an s-butyl group, or a t-butyl group, more preferably a methyl group, an ethyl group, an isopropyl group, or a t-butyl group, and further preferably a methyl group or a t-butyl group.

The unsubstituted aryl group represented by R¹⁰¹ to R¹⁰⁵ that are not a single bond, R¹¹¹ to R¹¹⁵, and R¹²¹ to R¹²⁵ is preferably a phenyl group, a biphenyl group, or a naphthyl group, and more preferably a phenyl group.

The unsubstituted heterocyclic group represented by R¹¹¹ to R¹¹⁵ and R¹²¹ to R¹²⁵ is preferably a pyridyl group or a quinazolinyl group.

In the formula (1), at least one of the hydrogen atoms contained in L, R¹¹ to R¹⁵ that are not a single bond, R²¹ to R²⁶ that are not a single bond, R⁵¹ to R⁶² that are not a single bond, R⁷¹ to R⁸⁰ that are not a single bond, R⁹¹ to R⁹⁸ that are not a single bond, R^{A} to R^{B}, and R¹⁰¹ to R¹⁰⁵ that are not a single bond is a deuterium atom.

In other words, the compound represented by the formula (1) has a structure in which at least one of the hydrogen atoms contained in L, R¹¹ to R¹⁵ that are not a single bond, R²¹ to R²⁶ that are not a single bond, R⁵¹ to R⁶² that are not a single bond, R⁷¹ to R⁸⁰ that are not a single bond, R⁹¹ to R⁹⁸ that are not a single bond, R^{A} to R^{B}, and R¹⁰¹ to R¹⁰⁵ that are not a single bond is a deuterium atom.

The deuterium atom contained in the inventive compound (1) will be described in detail later.

The inventive compound (1), for example, the inventive compound (1) is represented by any of the following formulae (1A) to (1C).

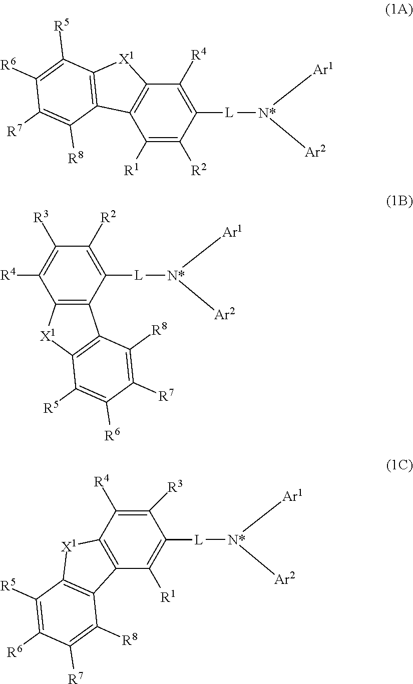

In the formula (1A), the formula (1B), and the formula (1C), Ar¹, Ar², L, N*, R¹ to R⁸, and X¹ are as defined in the formula (1).

In an aspect, the inventive compound (1) is represented by the following formulae (1Ab-1) to (1Ab-3), (1Bb-1) to (1Bb-3), or (1Cb-1) to (1Cb-3), and preferably represented by the following formula (1Ab-1), (1Bb-1), or (1Cb-1).

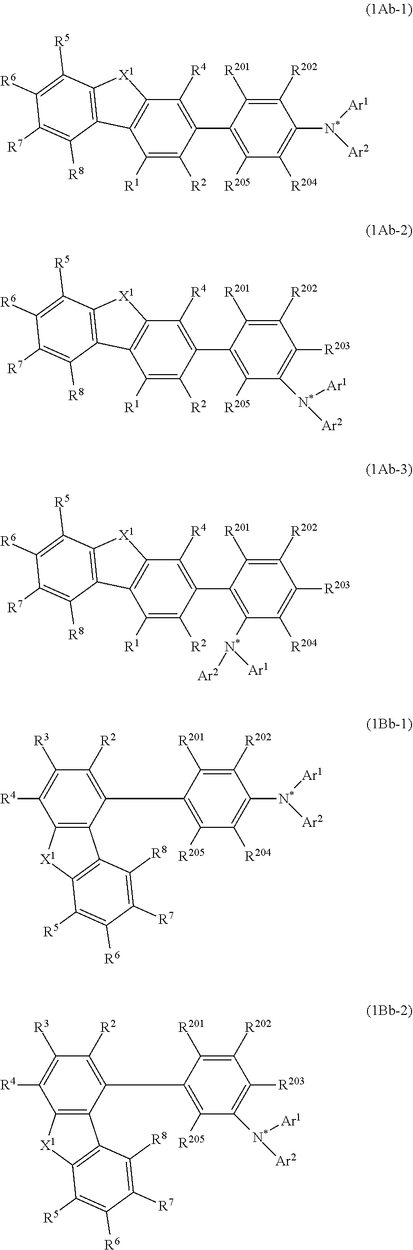

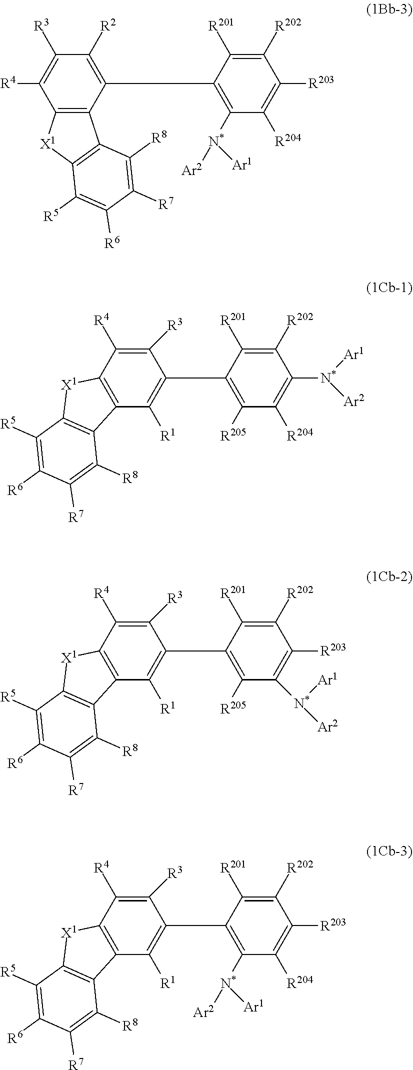

In the formula (1Ab-1) to the formula (1Ab-3), the formula (1Bb-1) to the formula (1Bb-3), and the formula (1Cb-1) to the formula (1Cb-3), Ar¹, Ar², N*, R¹ to R⁸, and X¹ are as defined in the formula (1). R²⁰¹ to R²⁰⁵ are each a hydrogen atom.

In compound (1), the combinations of one of Ar¹ and Ar² and the other of Ar¹ and Ar² include
the formula (1-a) and the formula (1-a),
the formula (1-a) and the formula (1-b),
the formula (1-a) and the formula (1-c),
the formula (1-a) and the formula (1-d),
the formula (1-a) and the formula (1-e),
the formula (1-a) and the formula (1-f),
the formula (1-b) and the formula (1-b),
the formula (1-b) and the formula (1-c),
the formula (1-b) and the formula (1-d),
the formula (1-b) and the formula (1-e),
the formula (1-b) and the formula (1-f),
the formula (1-c) and the formula (1-c),
the formula (1-c) and the formula (1-d),
the formula (1-c) and the formula (1-e),
the formula (1-c) and the formula (1-f),
the formula (1-d) and the formula (1-d),
the formula (1-d) and the formula (1-e),
the formula (1-d) and the formula (1-f),
the formula (1-e) and the formula (1-e),
the formula (1-e) and the formula (1-f), and,
the formula (1-f) and the formula (1-f).

Among them, a combination of
the formula (1-a) and the formula (1-a),
the formula (1-a) and the formula (1-b),
the formula (1-a) and the formula (1-c),
the formula (1-a) and the formula (1-e),
the formula (1-a) and the formula (1-f),
the formula (1-b) and the formula (1-b),
the formula (1-c) and the formula (1-e), or,
the formula (1-e) and the formula (1-e) is preferred.

The inventive compound (1) is, for example, represented by any of the following formulae (TA-3) to (1C-3).

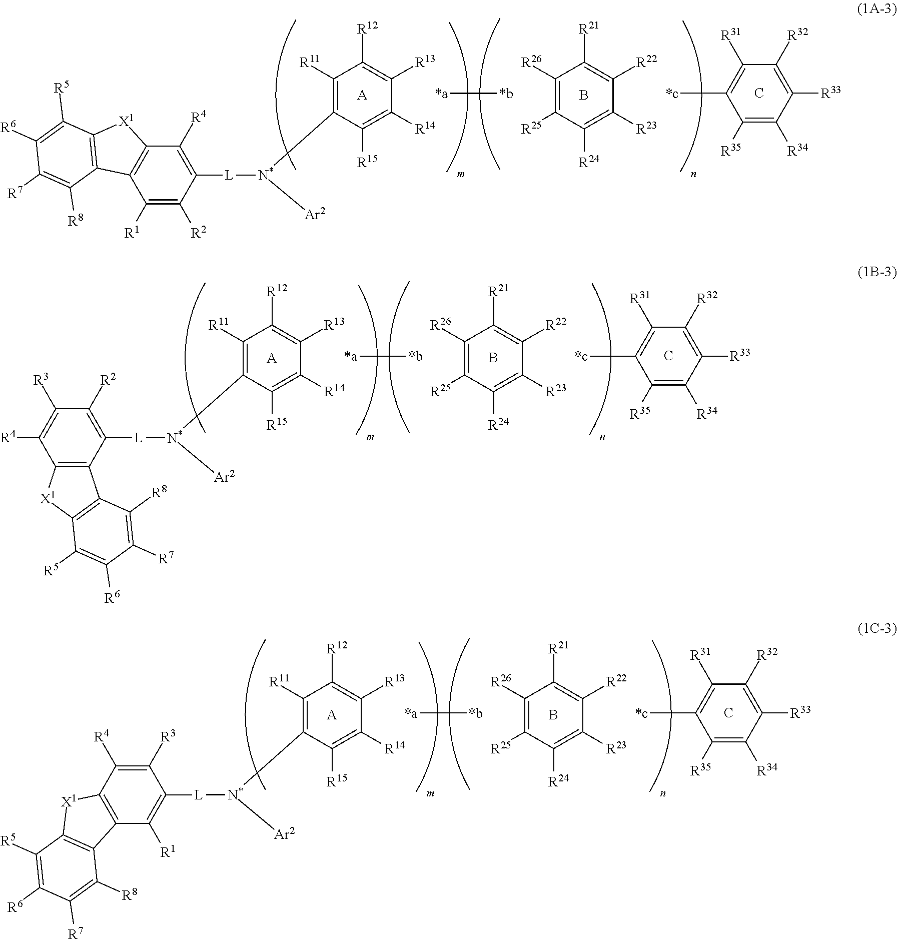

In the formula (1A-3), the formula (1B-3), and the formula (1C-3), Ar², L, N*, R¹ to R⁸, R¹¹ to R¹⁵, R²¹ to R²⁶, R³ to R³⁵, X¹, *a, *b, *c, m, and n are as defined in the formula (1).

The inventive compound (1) is, for example, represented by any of the following formulae (1A-4) to (1C-4).

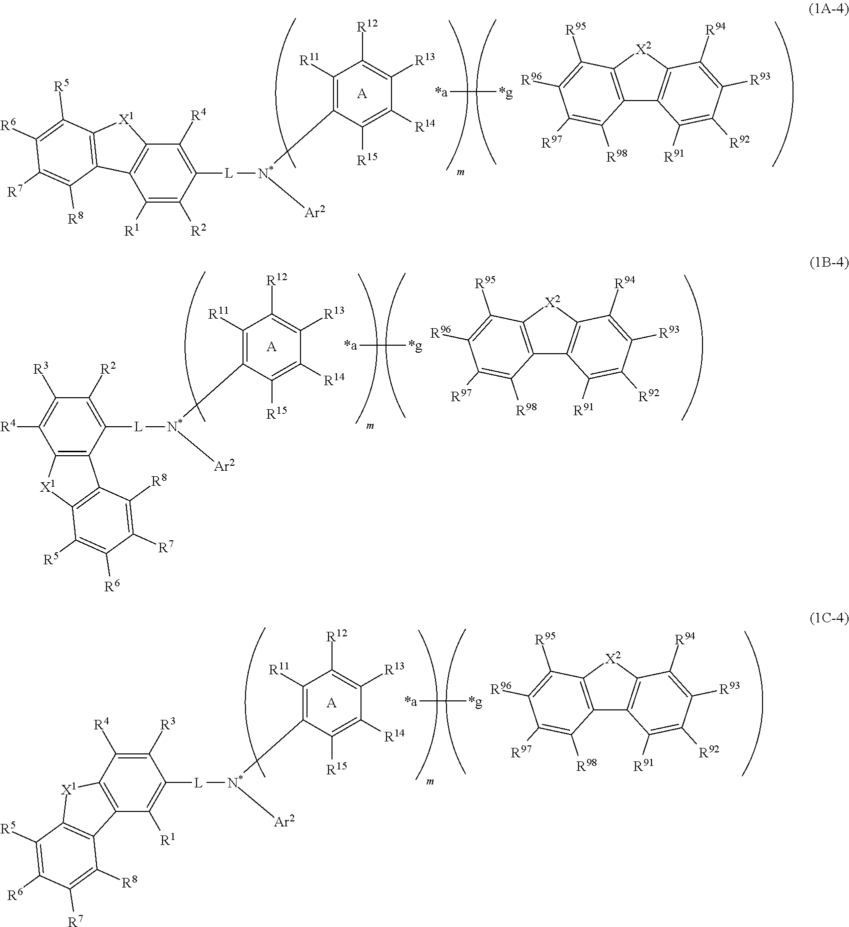

In the formula (1A-4), the formula (1B-4), and the formula (1C-4), Ar², L, N*, R¹ to R⁸, R¹¹ to R¹⁵, R⁹¹ to R⁹⁸, X¹, X², *a, *g, and m are as defined in the formula (1).

The inventive compound (1) is preferably represented by any of the following formulae (1-1) to (1-8), and more preferably represented by any of the following formulae (1-1) to (1-5).

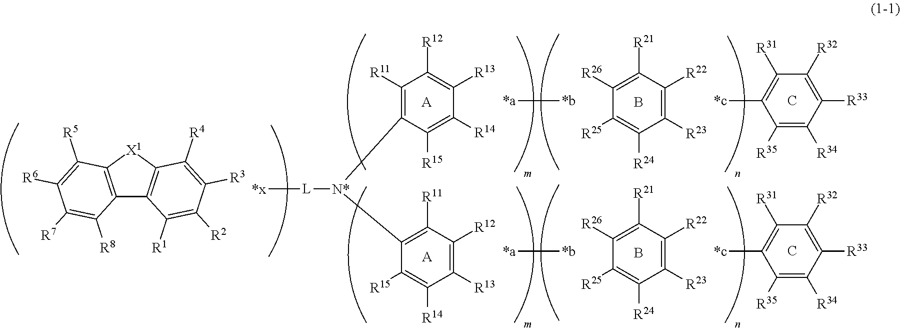

In the formula (1-1),
N*, X¹, R¹ to R⁸, *x, L, *a, *b, *c, R¹¹ to R¹⁵, R²¹ to R²⁶, R³¹ to R³⁵, m, and n are as defined in the formula (1) and the formula (1-a).

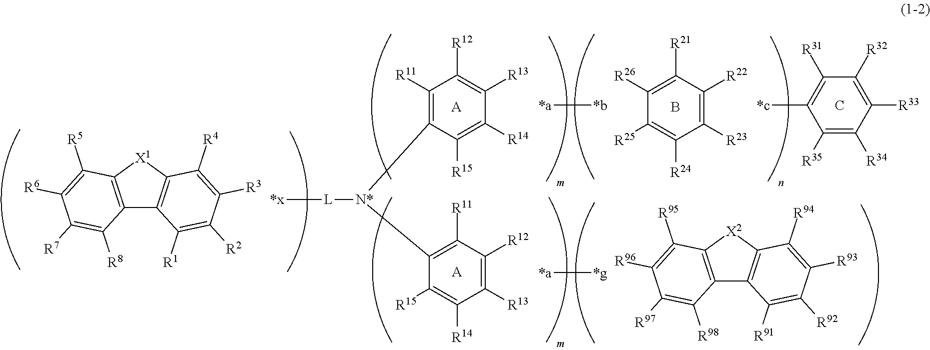

In the formula (1-2),
N*, X¹, R¹ to R⁸, *x, L, *a, *b, *c, *g, R¹¹ to R¹⁵, R²¹ to R²⁶, R³¹ to R³⁵, R⁹¹ to R⁹⁸, X², m, and n are as defined in the formula (1), the (1-a), and the formula (1-e).

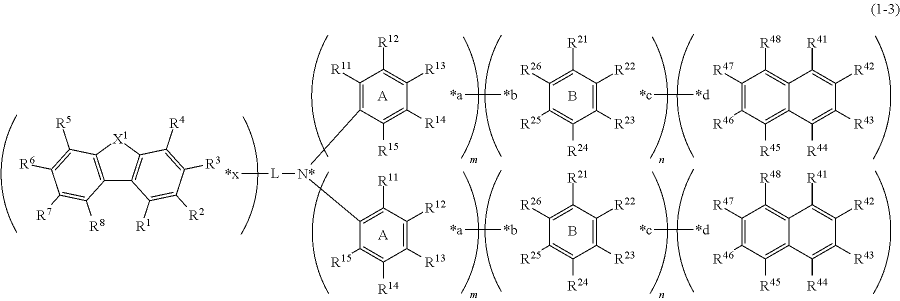

In the formula (1-3),
N*, X¹, R¹ to R⁸, *x, L, *a, *b, *c, *d, R¹¹ to R¹⁵, R²¹ to R²⁶, R⁴¹ to R⁴⁸, m, and n are as defined in the formula (1) and the formula (1-b).

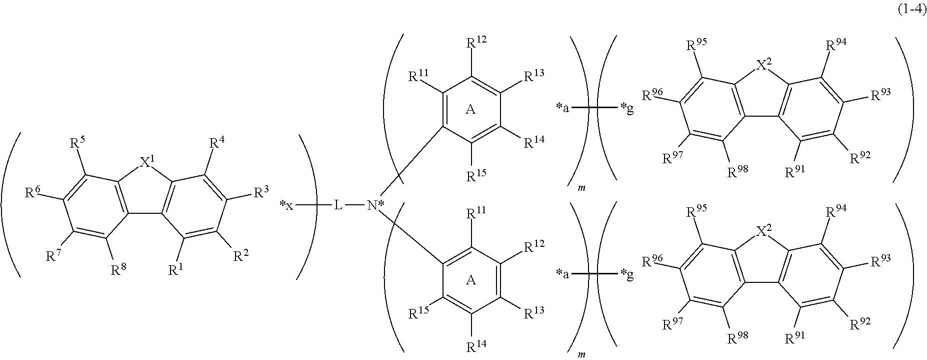

In the formula (1-4),
N*, X¹, R¹ to R⁸, *x, L, *a, *g, R¹¹ to R¹⁵, R⁹¹ to R⁹⁸, X², and m are as defined in the formula (1) and the formula (1-e).

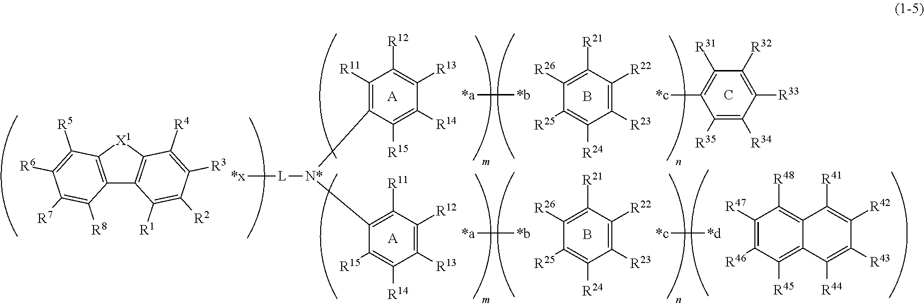

In the formula (1-5),
N*, X¹, R¹ to R⁸, *x, L, *a, *b, *c, *d, R¹¹ to R¹⁵, R²¹ to R²⁶, R³¹ to R³⁵, R⁴¹ to R⁴⁸, m, and n are as defined in the formula (1), the formula (1-a), and the formula (1-b).

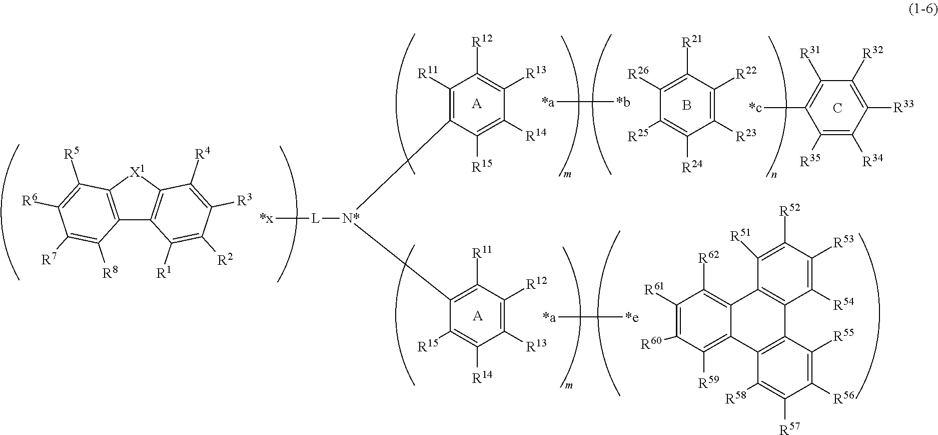

In the formula (1-6),
N*, X¹, R¹ to R⁸, *x, L, *a, *b, *c, *e, R¹¹ to R¹⁵, R²¹ to R²⁶, R³¹ to R³⁵, R⁵¹ to R⁶², m, and n are as defined in the formula (1), the formula (1-a), and the formula (1-c).

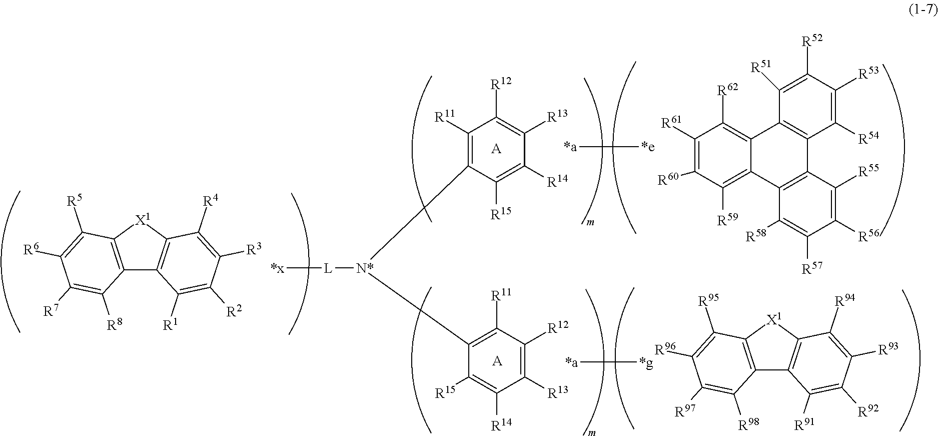

In the formula (1-7),
N*, X¹, R¹ to R⁸, *x, L, *a, *e, *g, R¹¹ to R¹⁵, R⁵¹ to R⁶², R⁹¹ to R⁹⁸, X², and m are as defined in the formula (1), the formula (1-c), and the formula (1-e).

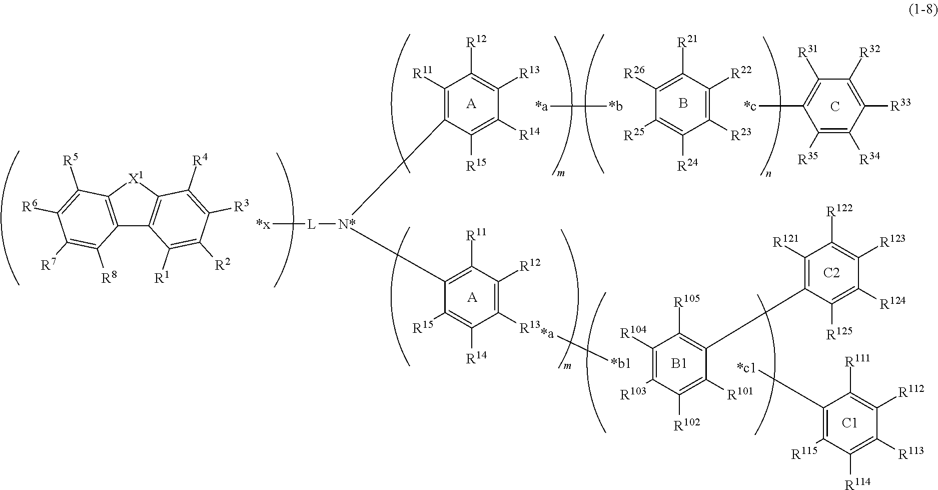

In the formula (1-8),
N*, X¹, R¹ to R⁸, *x, L, *a, *b, *c, *b1, *c1, R¹¹ to R¹⁵, R²¹ to R²⁶, R³¹ to R³⁵, R¹⁰¹ to R¹⁰⁵, R¹¹¹ to R¹¹⁵, R¹²¹ to R¹²⁵, m, and n are as defined in the formula (1), the formula (1-a), and the formula (1-f).

The inventive compound (1) is, for example, represented by any of the following formulae (1-1-A) to (1-8-C).

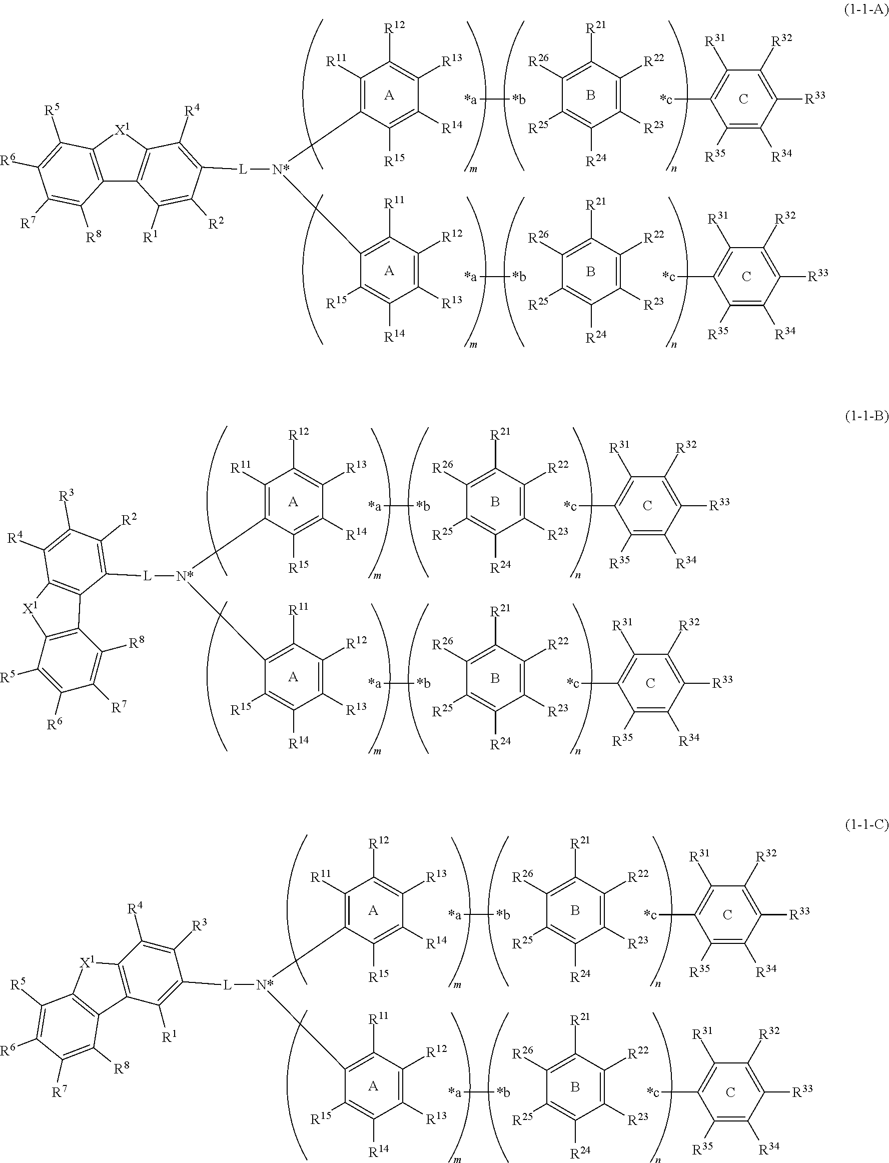

In the formula (1-1-A), the formula (1-1-B), and the formula (1-1-C),
N*, X¹, R¹ to R⁸, L, *a, *b, *c, R¹¹ to R¹⁵, R²¹ to R²⁶, R³¹ to R³⁵, m, and n are as defined in the formula (1) and the formula (1-a).

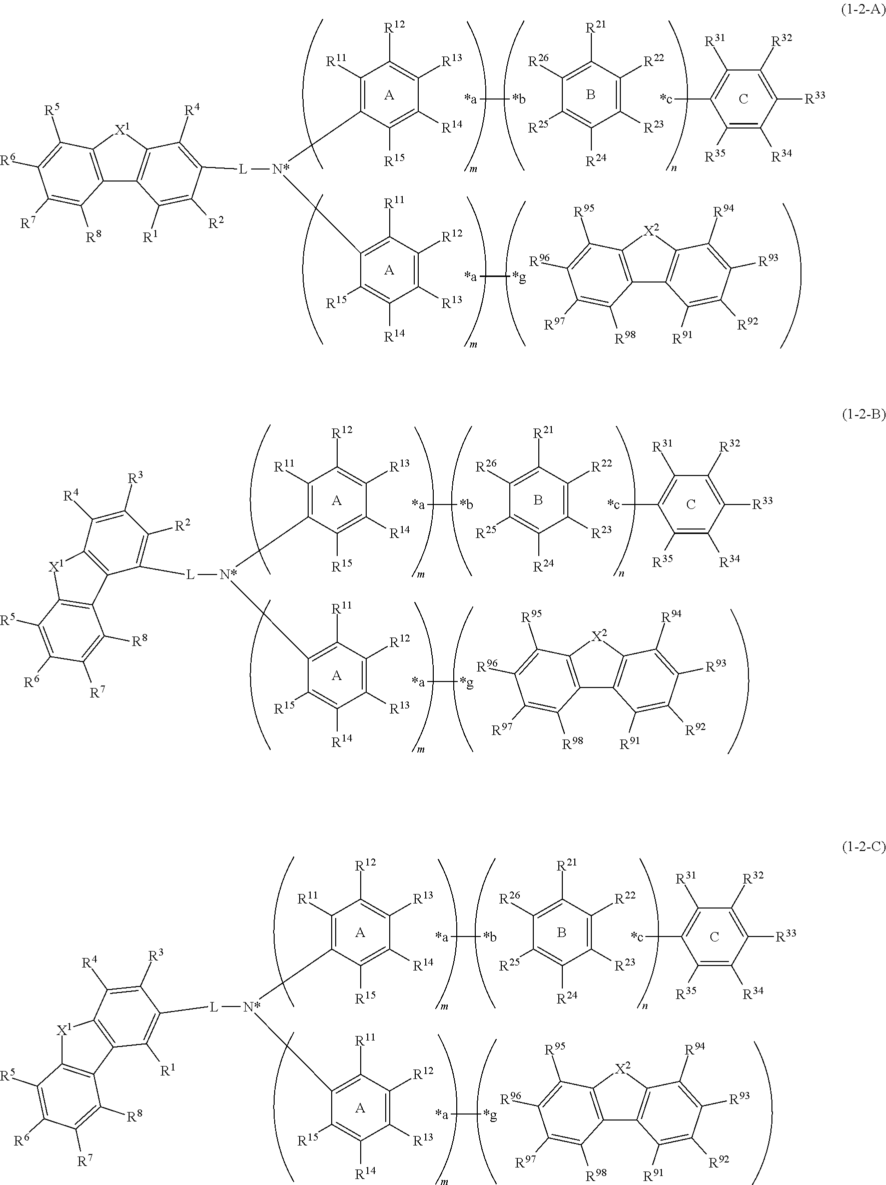

In the formula (1-2-A), the formula (1-2-B), and the formula (1-2-C),
N*, X¹, R¹ to R⁸, L, *a, *b, *C *g R¹¹ to R¹⁵, R²¹ to R²⁶, R³¹ to R³⁵, R⁹¹ to R⁹⁸, X², m, and n are as defined in the formula (1), the (1-a), and the formula (1-e).

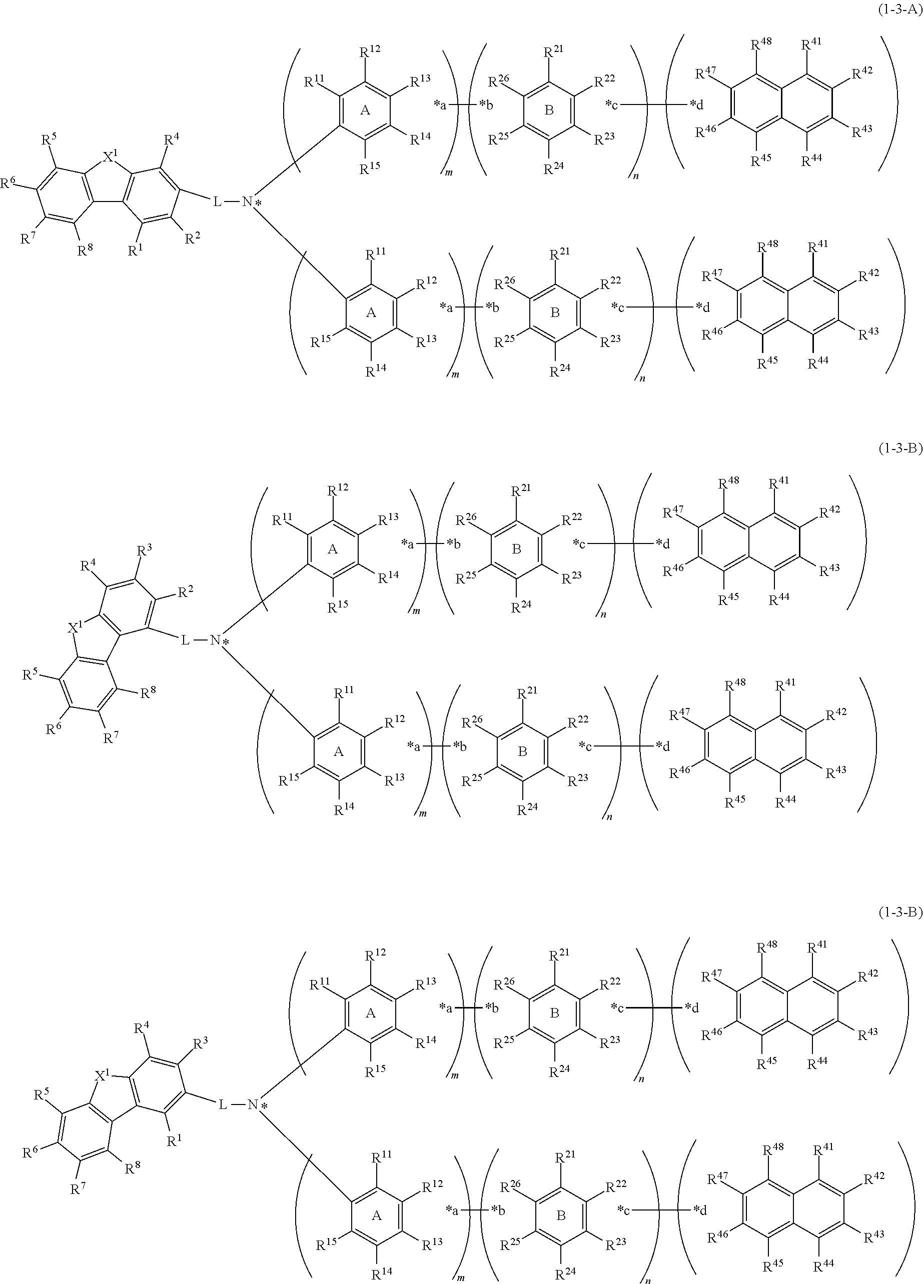

In the formula (1-3-A), the formula (1-3-B), and the formula (1-3-C),
N*, X¹, R¹ to R⁸, L, *a, *b, *c, *d, R¹¹ to R¹⁵, R²¹ to R²⁶, R⁴¹ to R⁴⁸, m, and n are as defined in the formula (1) and the formula (1-b).

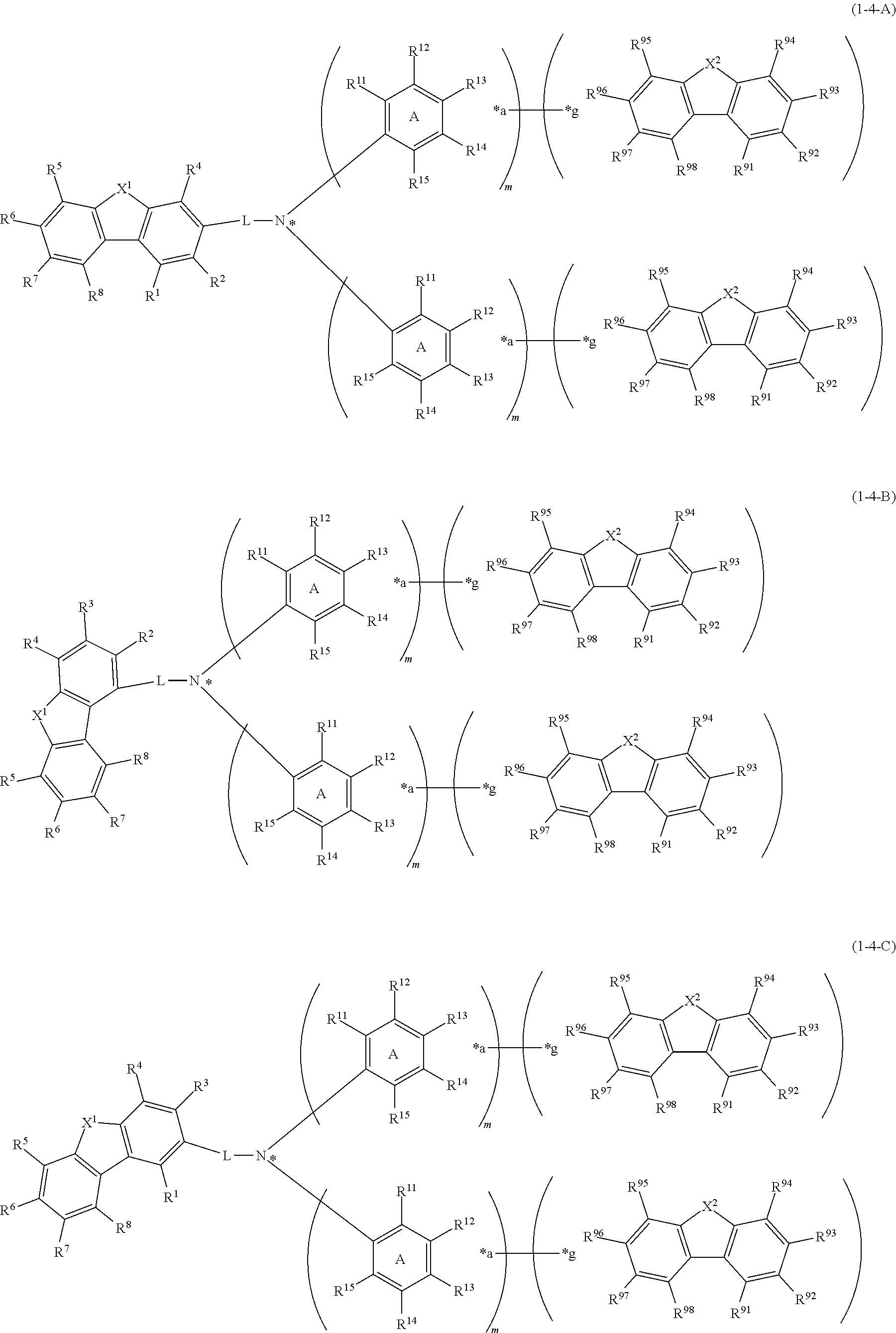

In the formula (1-4-A), the formula (1-4-B), and the formula (1-4-C),
N*, X¹, R¹ to R⁸, L, *a, *g, R¹¹ to R¹⁵, R⁹¹ to R⁹⁸, X², and m are as defined in the formula (1) and the formula (1-e).

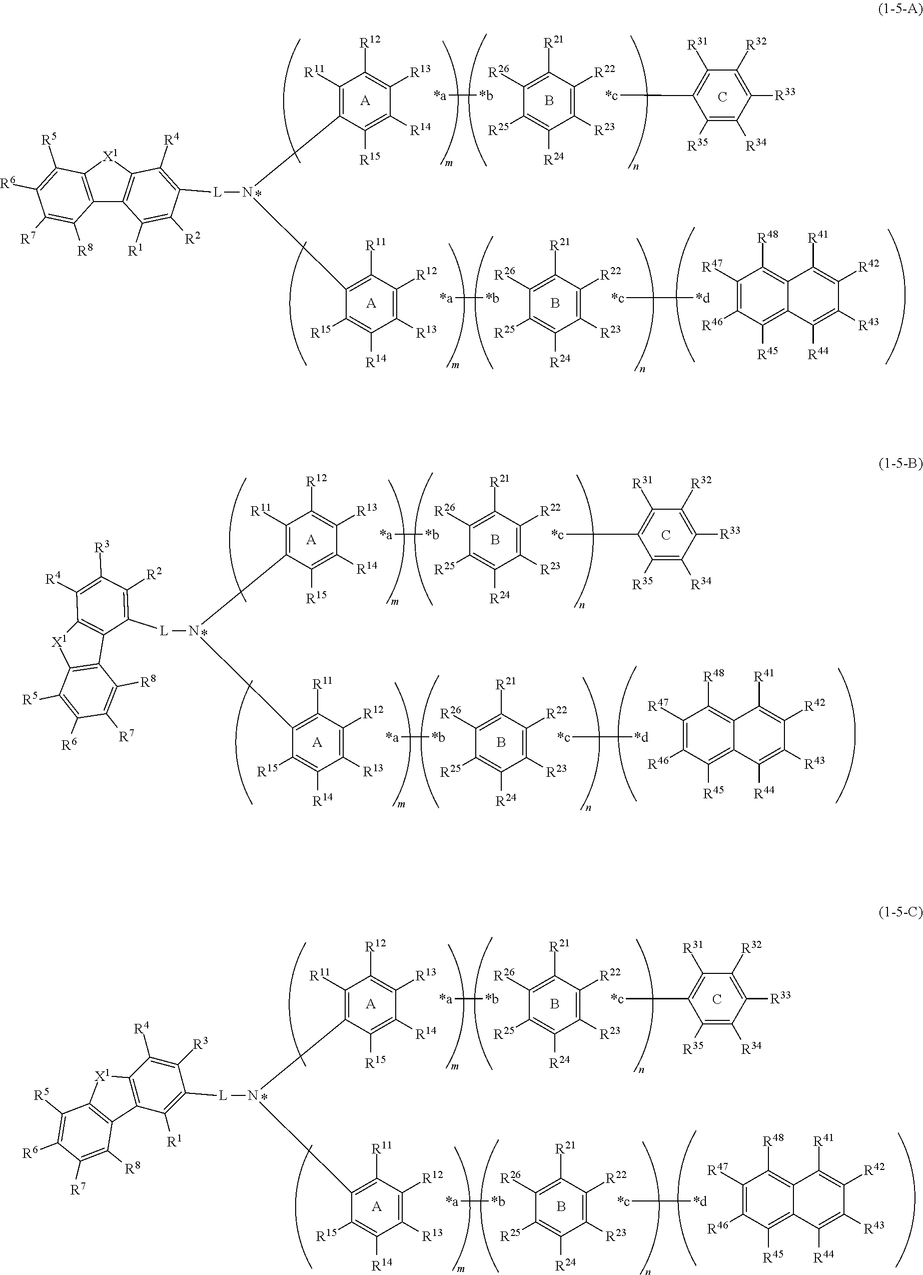

In the formula (1-5-A), the formula (1-5-B), and the formula (1-5-C),
N*, X¹, R¹ to R⁸, L, *a, *b, *c, *d, R¹¹ to R¹⁵, R²¹ to R²⁶, R³ to R³⁵, R⁴¹ to R⁴⁸, m, and n are as defined in the formula (1), the formula (1-a), and the formula (1-b).

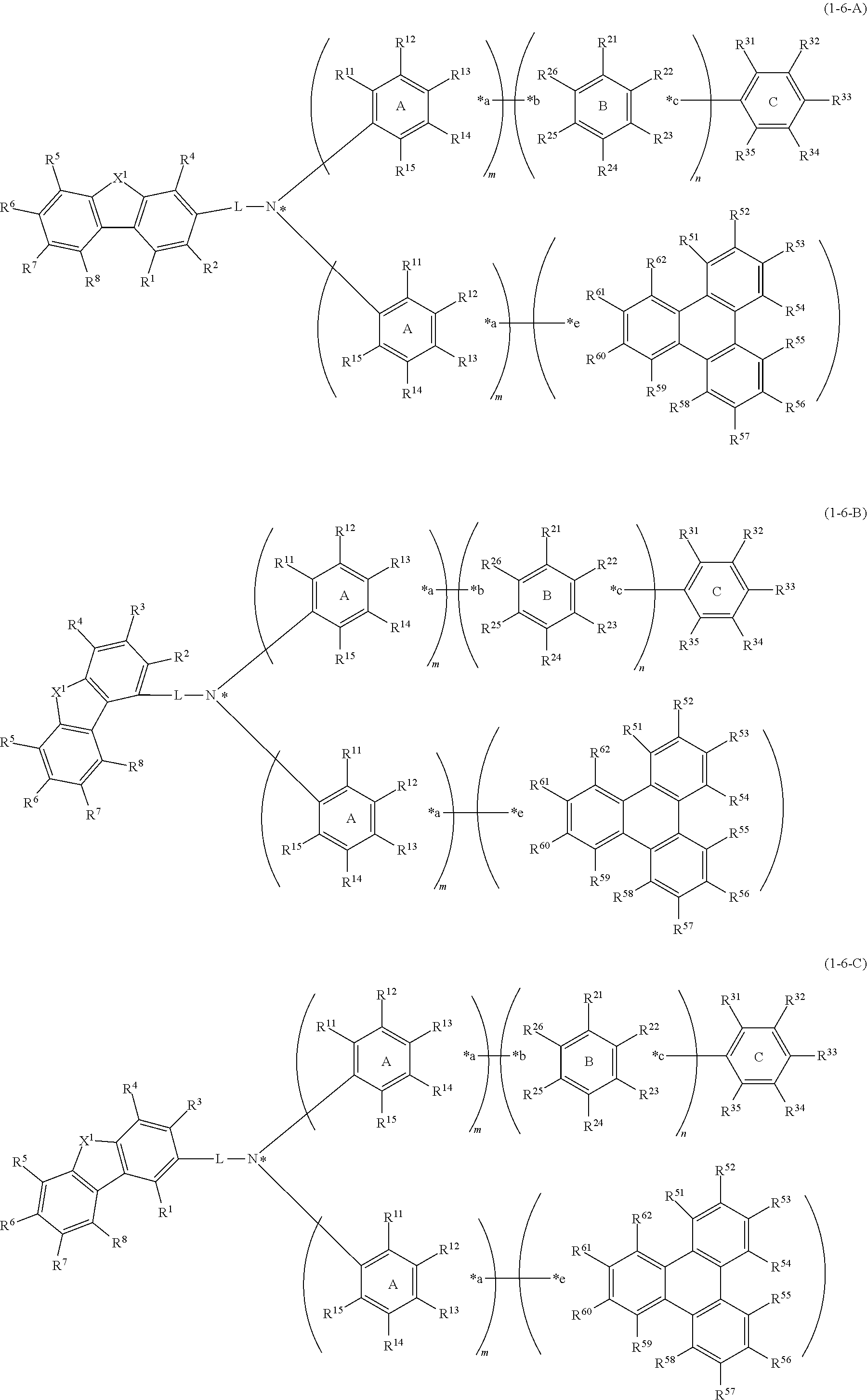

In the formula (1-6-A), the formula (1-6-B), and the formula (1-6-C),
N*, X¹, R¹ to R⁸, L, *a, *b, *c, *e, R¹¹ to R¹⁵, R²¹ to R²⁶, R³¹ to R³⁵, R⁵¹ to R⁶², m, and n are as defined in the formula (1), the formula (1-a), and the formula (1-c).

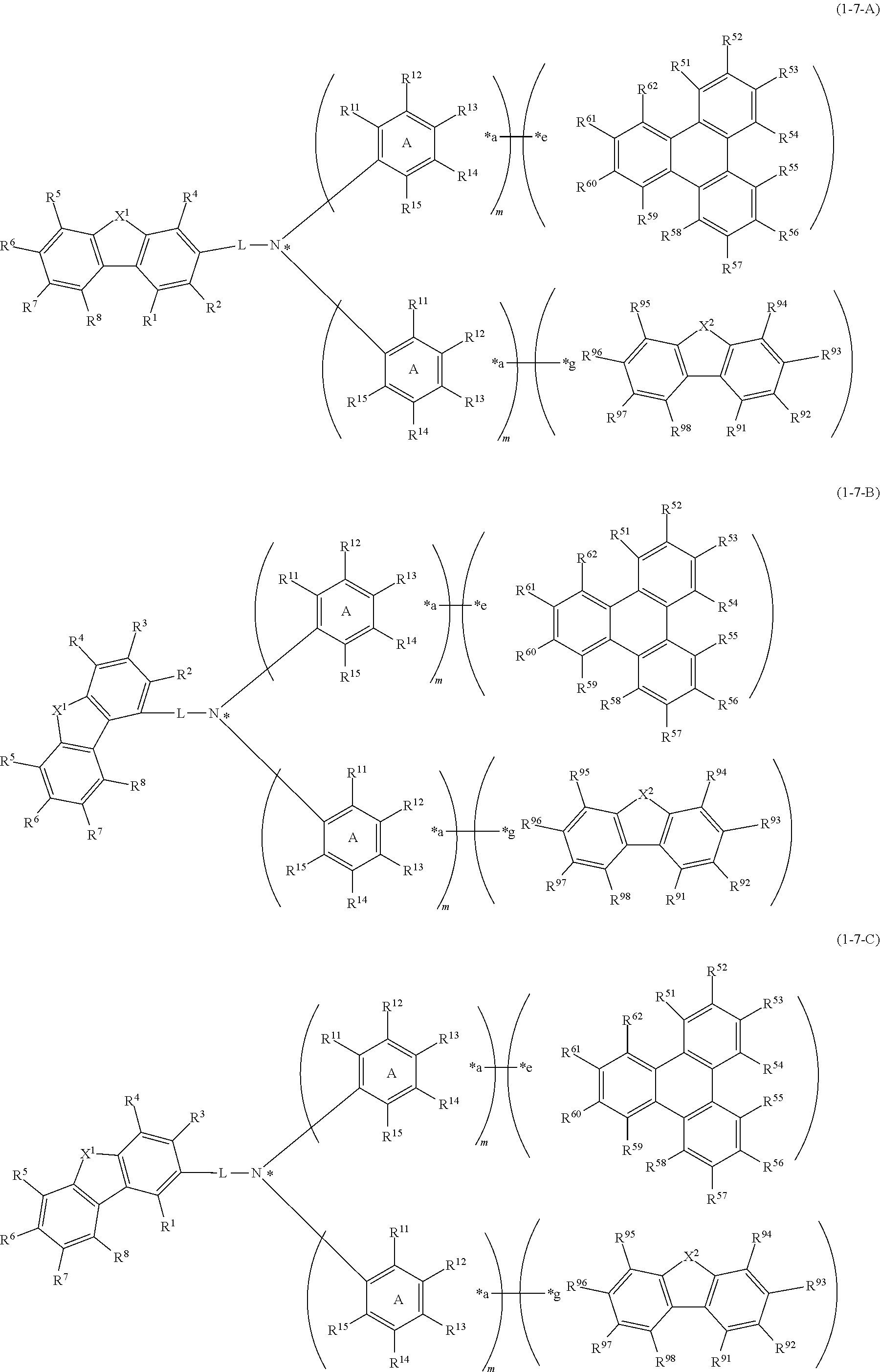

In the formula (1-7-A), the formula (1-7-B), and the formula (1-7-C),
N*, X¹, R¹ to R⁸, L, *a, *e, *g, R¹¹ to R¹⁵, R¹¹ to R⁶², R⁹¹ to R⁹⁸, X², and m are as defined in the formula (1), the formula (1-c), and the formula (1-e).

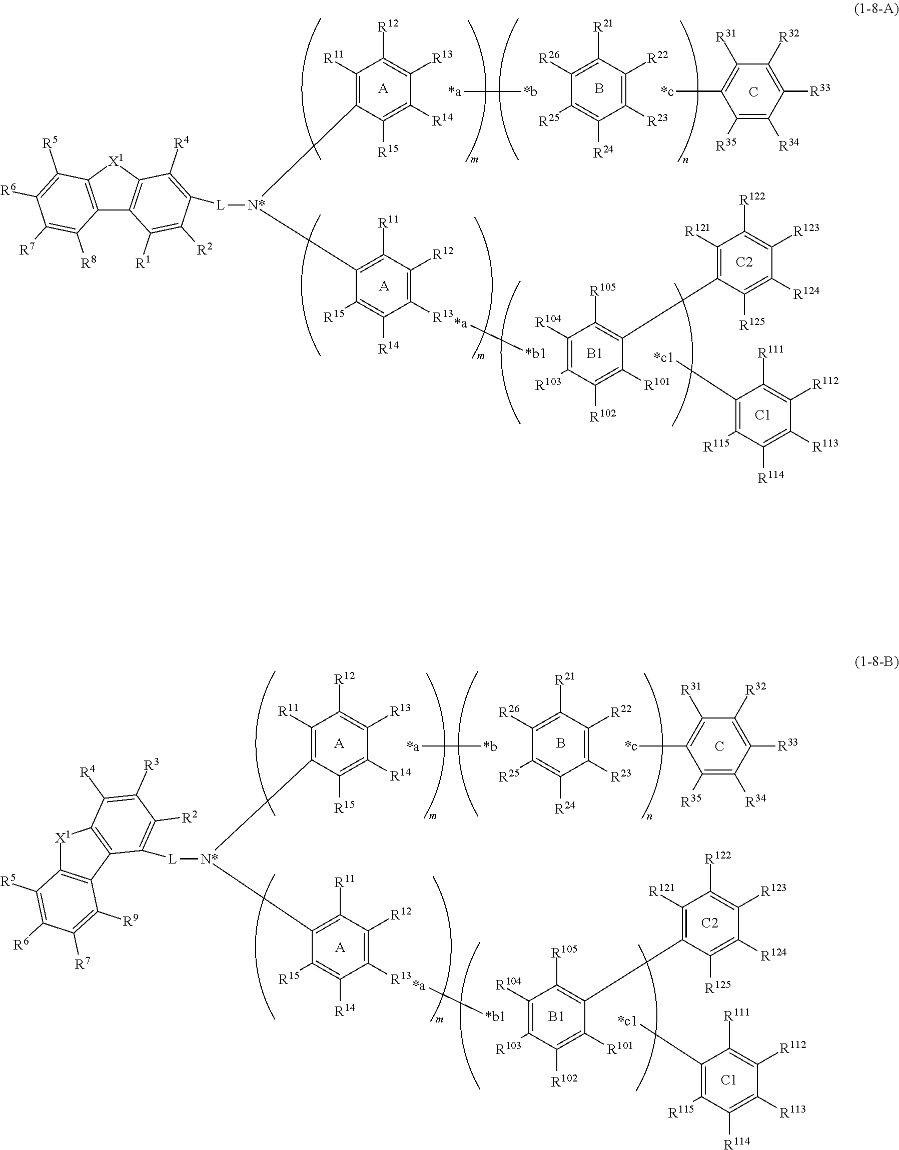

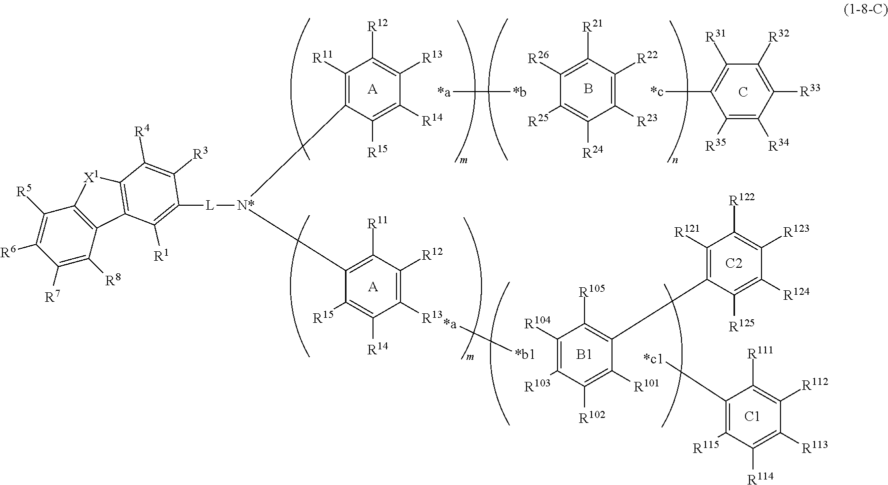

In the formula (1-8-A), the formula (1-8-B) and the formula (1-8-C),
N*, X¹, R¹ to R⁸, L, *a, *b, *c, *b1, *c1, R¹¹ to R¹⁵, R²¹ to R²⁶, R³¹ to R³⁵, R¹⁰¹ to R¹⁰⁵, R¹¹¹ to R¹¹⁵, R¹²¹ to R¹²⁵, m, and n are as defined in the formula (1), the formula (1-a), and the formula (1-f).

In an aspect of the inventive compound (1), at least one of the following (1) to (15) is a deuterium atom:
(1) a hydrogen atom directly bonded to the phenylene group represented by L;
(2) a hydrogen atom represented by R¹¹ to R¹⁵ that are not a single bond, a hydrogen atom represented by R²¹ to R²⁶ that are not a single bond, a hydrogen atom represented by R⁵¹ to R⁶² that are not a single bond, a hydrogen atom represented by R⁷¹ to R⁸⁰ that are not a single bond, a hydrogen atom represented by R⁹¹ to R⁹⁸ that are not a single bond, and a hydrogen atom represented by R¹⁰¹ to R¹⁰⁵ that are not a single bond, which are contained in Ar¹;
(3) a hydrogen atom directly bonded to an alkyl group represented by R¹¹ to R¹⁵ that are not a single bond, a hydrogen atom directly bonded to an alkyl group represented by R²¹ to R²⁶ that are not a single bond, a hydrogen atom directly bonded to an alkyl group represented by R⁵¹ to R⁶² that are not a single bond, a hydrogen atom directly bonded to an alkyl group represented by R⁷¹ to R⁸⁰ that are not a single bond, a hydrogen atom directly bonded to an alkyl group represented by R⁹¹ to R⁹⁸ that are not a single bond, a hydrogen atom directly bonded to an alkyl group represented by R^{A} to R^{B}, and a hydrogen atom directly bonded to an alkyl group represented by R¹⁰¹ to R¹⁰⁵ that are not a single bond, which are contained in Ar¹;
(4) a hydrogen atom directly bonded to a substituent on an alkyl group represented by R¹¹ to R¹⁵ that are not a single bond, a hydrogen atom directly bonded to a substituent on an alkyl group represented by R²¹ to R²⁶ that are not a single bond, a hydrogen atom directly bonded to a substituent on an alkyl group represented by R⁵¹ to R⁶² that are not a single bond, a hydrogen atom directly bonded to a substituent on an alkyl group represented by R⁷¹ to R⁸⁰ that are not a single bond, a hydrogen atom directly bonded to a substituent on an alkyl group represented by R⁹¹ to R⁹⁸ that are not a single bond, a hydrogen atom directly bonded to a substituent on an alkyl group represented by R^{A} to R^{B}, and a hydrogen atom directly bonded to a substituent on an alkyl group represented by R¹⁰¹ to R¹⁰⁵ that are not a single bond, which are contained in Ar¹;
(5) a hydrogen atom directly bonded to an aryl group represented by R¹¹ to R¹⁵ that are not a single bond, a hydrogen atom directly bonded to an aryl group represented by R²¹ to R²⁶ that are not a single bond, a hydrogen atom directly bonded to an aryl group represented by R⁵¹ to R⁶² that are not a single bond, a hydrogen atom directly bonded to an aryl group represented by R⁷¹ to R⁸⁰ that are not a single bond, a hydrogen atom directly bonded to an aryl group represented by R⁹¹ to R⁹⁸ that are not a single bond, a hydrogen atom directly bonded to an aryl group represented by R^{A} to R^{B}, and a hydrogen atom directly bonded to an aryl group represented by R¹⁰¹ to R¹⁰⁵ that are not a single bond, which are contained in Ar¹;
(6) a hydrogen atom directly bonded to a substituent on an aryl group represented by R¹¹ to R¹⁵ that are not a single bond, a hydrogen atom directly bonded to a substituent on an aryl group represented by R²¹ to R²⁶ that are not a single bond, a hydrogen atom directly bonded to a substituent on an aryl group represented by R⁵¹ to R⁶² that are not a single bond, a hydrogen atom directly bonded to a substituent on an aryl group represented by R⁷¹ to R⁸⁰ that are not a single bond, a hydrogen atom directly bonded to a substituent on an aryl group represented by R⁹¹ to R⁹⁸ that are not a single bond, a hydrogen atom directly bonded to a substituent on an aryl group represented by R^{A} to R^{B}, and a hydrogen atom directly bonded to a substituent on an aryl group represented by R¹⁰¹ to R¹⁰⁵ that are not a single bond, which are contained in Ar¹;
(7) a hydrogen atom directly bonded to a heterocyclic group represented by R⁵¹ to R⁶² that are not a single bond, a hydrogen atom directly bonded to a heterocyclic group represented by R⁷¹ to R⁸⁰ that are not a single bond, and a hydrogen atom directly bonded to a heterocyclic group represented by R⁹¹ to R⁹⁸ that are not a single bond, which are contained in Ar¹;
(8) a hydrogen atom directly bonded to a substituent on a heterocyclic group represented by R⁵¹ to R⁶² that are not a single bond, a hydrogen atom directly bonded to a substituent on a heterocyclic group represented by R⁷¹ to R⁸⁰ that are not a single bond, and a hydrogen atom directly bonded to a substituent on a heterocyclic group represented by R⁹¹ to R⁹⁸ that are not a single bond, which are contained in Ar¹;
(9) a hydrogen atom represented by R¹¹ to R¹⁵ that are not a single bond, a hydrogen atom represented by R²¹ to R²⁶ that are not a single bond, a hydrogen atom represented by R⁵¹ to R⁶² that are not a single bond, a hydrogen atom represented by R⁷¹ to R⁸⁰ that are not a single bond, a hydrogen atom represented by R⁹¹ to R⁹⁸ that are not a single bond, and a hydrogen atom represented by R¹⁰¹ to R¹⁰⁵ that are not a single bond, which are contained in Ar²;
(10) a hydrogen atom directly bonded to an alkyl group represented by R¹¹ to R¹⁵ that are not a single bond, a hydrogen atom directly bonded to an alkyl group represented by R²¹ to R²⁶ that are not a single bond, a hydrogen atom directly bonded to an alkyl group represented by R⁵¹ to R⁶² that are not a single bond, a hydrogen atom directly bonded to an alkyl group represented by R⁷¹ to R⁸⁰ that are not a single bond, a hydrogen atom directly bonded to an alkyl group represented by R⁹¹ to R⁹⁸ that are not a single bond, a hydrogen atom directly bonded to an alkyl group represented by R^{A} to R^{B}, and a hydrogen atom directly bonded to an alkyl group represented by R¹⁰¹ to R¹⁰⁵ that are not a single bond, which are contained in Ar²;
(11) a hydrogen atom directly bonded to a substituent on an alkyl group represented by R¹¹ to R¹⁵ that are not a single bond, a hydrogen atom directly bonded to a substituent on an alkyl group represented by R²¹ to R²⁶ that are not a single bond, a hydrogen atom directly bonded to a substituent on an alkyl group represented by R⁵¹ to R⁶² that are not a single bond, a hydrogen atom directly bonded to a substituent on an alkyl group represented by R⁷¹ to R⁸⁰ that are not a single bond, a hydrogen atom directly bonded to a substituent on an alkyl group represented by R⁹¹ to R⁹⁸ that are not a single bond, a hydrogen atom directly bonded to a substituent on an alkyl group represented by R^{A} to R^{B}, and a hydrogen atom directly bonded to a substituent on an alkyl group represented by R¹⁰¹ to R¹⁰⁵ that are not a single bond, which are contained in Ar²;
(12) a hydrogen atom directly bonded to an aryl group represented by R¹¹ to R¹⁵ that are not a single bond, a hydrogen atom directly bonded to an aryl group represented by R²¹ to R²⁶ that are not a single bond, a hydrogen atom directly bonded to an aryl group represented by R⁵¹ to R⁶² that are not a single bond, a hydrogen atom directly bonded to an aryl group represented by R⁷¹ to R⁸⁰ that are not a single bond, a hydrogen atom directly bonded to an aryl group represented by R⁹¹ to R⁹⁸ that are not a single bond, a hydrogen atom directly bonded to an aryl group represented by R^{A} to R^{B}, and a hydrogen atom directly bonded to an aryl group represented by R¹⁰¹ to R¹⁰⁵ that are not a single bond, which are contained in Ar²;
(13) a hydrogen atom directly bonded to a substituent on an aryl group represented by R¹¹ to R¹⁵ that are not a single bond, a hydrogen atom directly bonded to a substituent on an aryl group represented by R²¹ to R²⁶ that are not a single bond, a hydrogen atom directly bonded to a substituent on an aryl group represented by R⁵¹ to R⁶² that are not a single bond, a hydrogen atom directly bonded to a substituent on an aryl group represented by R⁷¹ to R⁸⁰ that are not a single bond, a hydrogen atom directly bonded to a substituent on an aryl group represented by R⁹¹ to R⁹⁸ that are not a single bond, a hydrogen atom directly bonded to a substituent on an aryl group represented by R^{A} to R^{B}, and a hydrogen atom directly bonded to a substituent on an aryl group represented by R¹⁰¹ to R¹⁰⁵ that are not a single bond, which are contained in Ar²;
(14) a hydrogen atom directly bonded to a heterocyclic group represented by R⁵¹ to R⁶² that are not a single bond, a hydrogen atom directly bonded to a heterocyclic group represented by R⁷¹ to R⁸⁰ that are not a single bond, and a hydrogen atom directly bonded to a heterocyclic group represented by R⁹¹ to R⁹⁸ that are not a single bond, which are contained in Ar²;
(15) a hydrogen atom directly bonded to a substituent on a heterocyclic group represented by R⁵¹ to R⁶² that are not a single bond, a hydrogen atom directly bonded to a substituent on a heterocyclic group represented by R⁷¹ to R⁸⁰ that are not a single bond, and a hydrogen atom directly bonded to a substituent on a heterocyclic group represented by R⁹¹ to R⁹⁸ that are not a single bond, which are contained in Ar².

In addition, the inventive compound (1) is, for example, represented by any of the following formulae (1F-1) to (1F-3), preferably represented by the following formula (1F-1) or (1F-2), and more preferably represented by the following formula (1F-1).

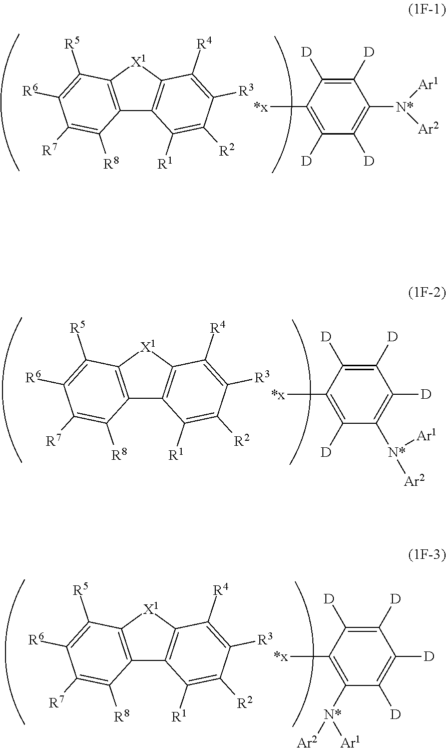

In the formula (1F-1), the formula (1F-2), and the formula (1F-3), Ar¹, Ar², N*, R¹ to R⁸, and *x are as defined in the formula (1).

In an aspect of the inventive compound (1), at least any of Ar¹, Ar², and R¹ to R⁸ in the formula (1F-1), the formula (1F-2), or the formula (1F-3) contains a deuterium atom, or none of them contains a deuterium atom.

In addition, the inventive compound (1) is, for example, represented by any of the following formulae (1G-1) to (1G-6), preferably represented by the following formula (1G-1), (1G-2), or (1G-5), and more preferably represented by the following formula (1G-1) or (1G-5).

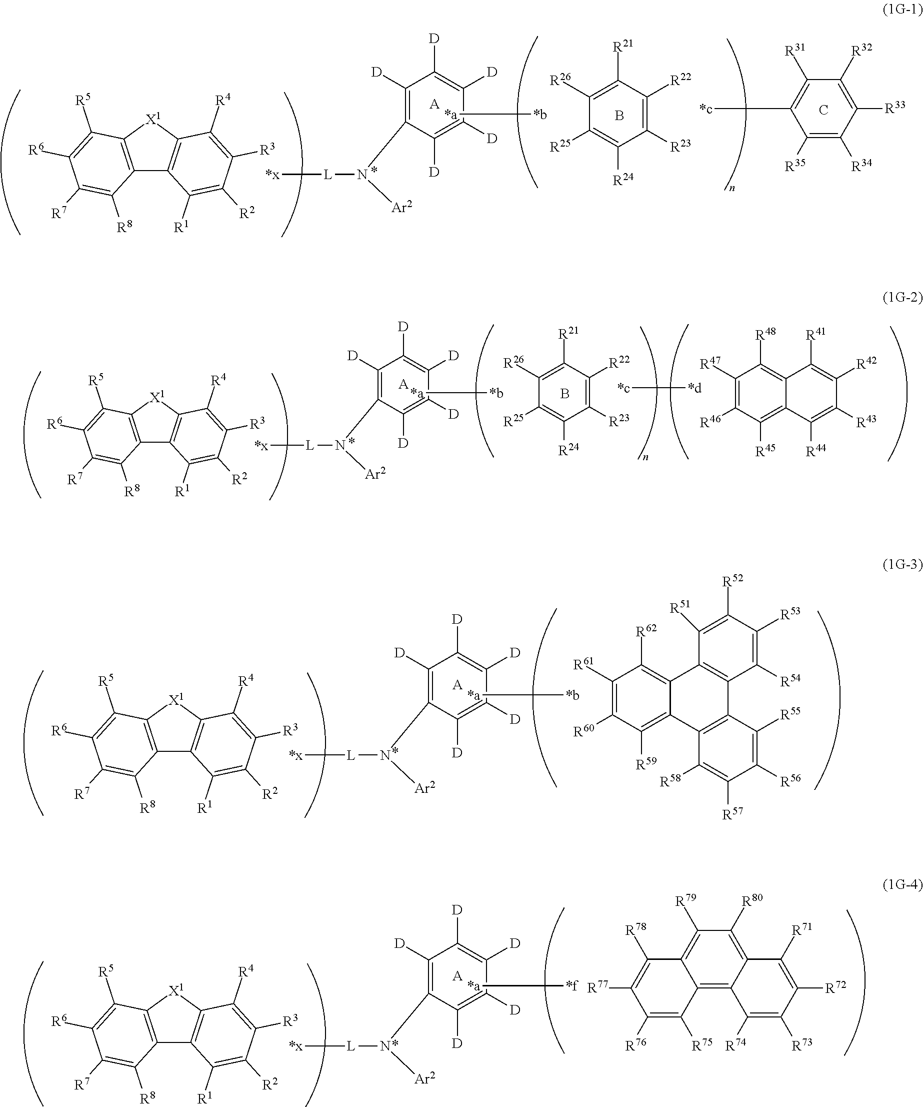

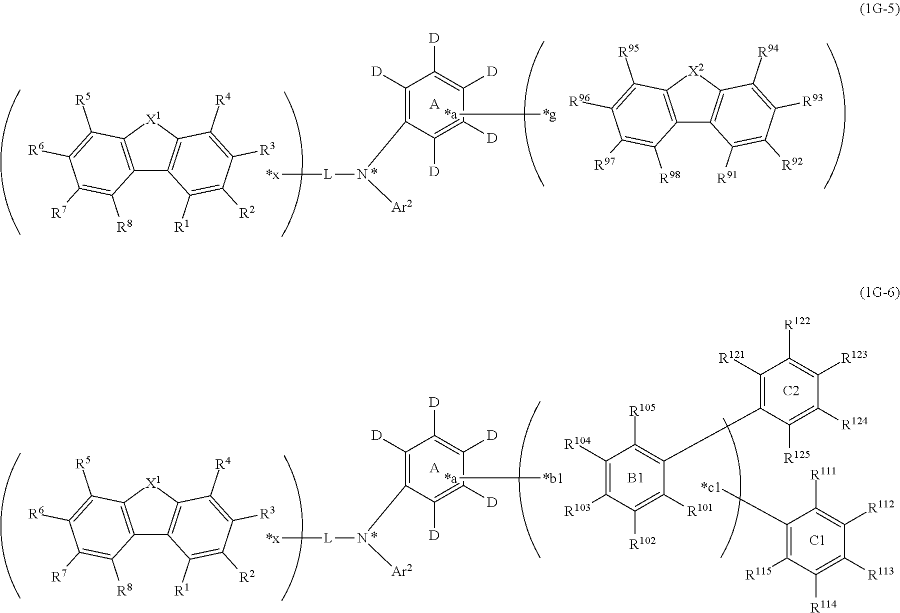

In the formula (1G-1), the formula (1G-2), the formula (1G-3), the formula (1G-4), the formula (1G-5), and the formula (1G-6), one of the carbon atoms that constitute a benzene ring A except for the carbon atom bonded to the central nitrogen atom N* is bonded to *a. In the formula (1G-1) to the formula (1G-6), Ar², L, N*, R¹ to R⁸, R²¹ to R²⁶, R³¹ to R³⁵, R⁴¹ to R⁴⁸, R⁵¹ to R⁶², R⁷¹ to R⁸⁰, R⁹¹ to R⁹⁸, R¹⁰¹ to R¹⁰⁵, R¹¹¹ to R¹¹⁵, R¹²¹ to R¹²⁵, X¹, X², *b, *b1, *c, *c1, *d, *f, *g, *x, and n are as defined in the formula (1).

In an aspect of the inventive compound (1), in the formula (1G-1), the formula (1G-2), the formula (1G-3), the formula (1G-4), the formula (1G-5), or the formula (1G-6), at least any of Ar², L, R¹ to R⁸, R²¹ to R²⁶, R³¹ to R³⁵, R⁴¹ to R⁴⁸, R⁵¹ to R⁶², R⁷¹ to R⁸⁰, R⁹¹ to R⁹⁸, R¹⁰¹ to R¹⁰⁵, R¹¹¹ to R¹¹⁵, X², and R¹²¹ to R¹²⁵ contains a deuterium atom or none of them contains a deuterium atom.

In the formula (1G-1) to the formula (1G-6), *a is bonded to the benzene ring A so as to be at the para position, the meta position, or the ortho position with respect to the central nitrogen atom N*, preferably bonded to the benzene ring A so as to be at the para position or the meta position with respect to the central nitrogen atom N*, and more preferably bonded to the benzene ring A so as to be at the para position with respect to the central nitrogen atom N*.

As described above, the “hydrogen atom” as used herein encompasses a protium atom, a deuterium atom, and a tritium atom. The inventive compound may contain a naturally occurring deuterium atom.

A deuterium atom may be intentionally introduced into the inventive compound by using a deuterated compound as a part or the whole of the raw material compounds.

The deuteration rate of the inventive compound depends on the deuteration rates of the raw material compounds used. Even when raw materials having prescribed deuteration rates are used, a protium isotope is possibly incorporated at a naturally derived certain proportion. Accordingly, in the aspects of the deuteration rate of the inventive compound shown below, a ratio involved in minute naturally occurring isotopes is included in addition to the proportion determined by simply counting the number of deuterium atoms represented by the chemical formula.

The deuteration rate of the inventive compound is preferably 1% or more, more preferably 3% or more, further preferably 5% or more, furthermore preferably 10% or more, and furthermore preferably 50% or more.

The inventive compound (1) may be a deuterium form in which all the hydrogen atoms are each a deuterium atom (that is, the inventive compound having a deuteration rate of 100%).

The inventive compound may be a mixture containing a deuterated compound and a non-deuterated compound or may be a mixture of two or more compounds having different deuteration rates. The deuteration rate of such a mixture is preferably 1% or more, more preferably 3% or more, further preferably 5% or more, furthermore preferably 10% or more, and furthermore preferably 50% or more, and less than 100%.

The proportion of the number of deuterium atoms with respect to the number of all the hydrogen atoms in the inventive compound is preferably 1% or more, more preferably 3% or more, further preferably 5% or more, and furthermore preferably 10% or more, and 100% or less.

Details of the substituent (optional substituent) in the “substituted or unsubstituted” included in the definition of each formula described above are as described in the section of “Substituent for “Substituted or Unsubstituted””.

The inventive compound can be easily produced by a person skilled in the art with reference to a synthetic example as described later and a known synthetic method.

Specific examples of the inventive compound will be shown below, but the inventive compound is not to be limited to the exemplified compounds.

In the specific examples, D represent a deuterium atom.

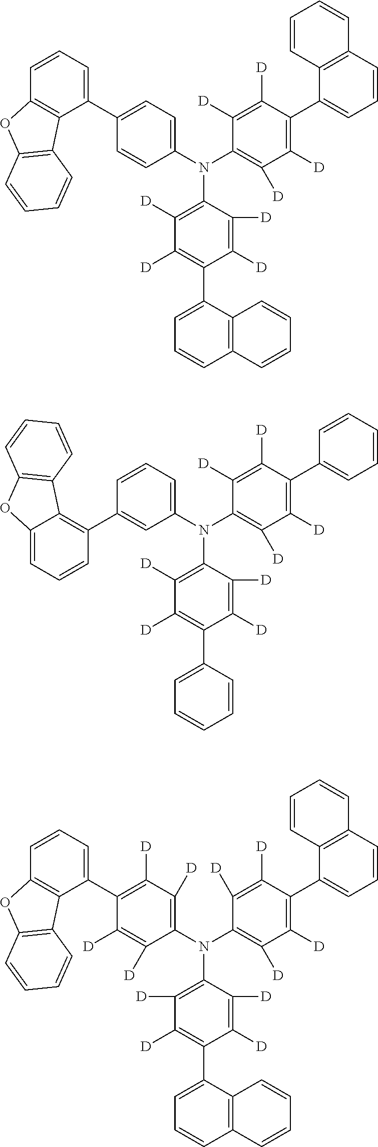 [SMILES: [2H]c1c([2H])c(N(c2ccc(-c3cccc4oc5ccccc5c34)cc2)c2c([2H])c([2H])c(-c3cccc4ccccc34)c([2H])c2[2H])c([2H])c([2H])c1-c1cccc2ccccc12 ; [2H]c1c([2H])c(N(c2cccc(-c3cccc4oc5ccccc5c34)c2)c2c([2H])c([2H])c(-c3ccccc3)c([2H])c2[2H])c([2H])c([2H])c1-c1ccccc1 ; [2H]c1c([2H])c(N(c2c([2H])c([2H])c(-c3cccc4ccccc34)c([2H])c2[2H])c2c([2H])c([2H])c(-c3cccc4oc5ccccc5c34)c([2H])c2[2H])c([2H])c([2H])c1-c1cccc2ccccc12]

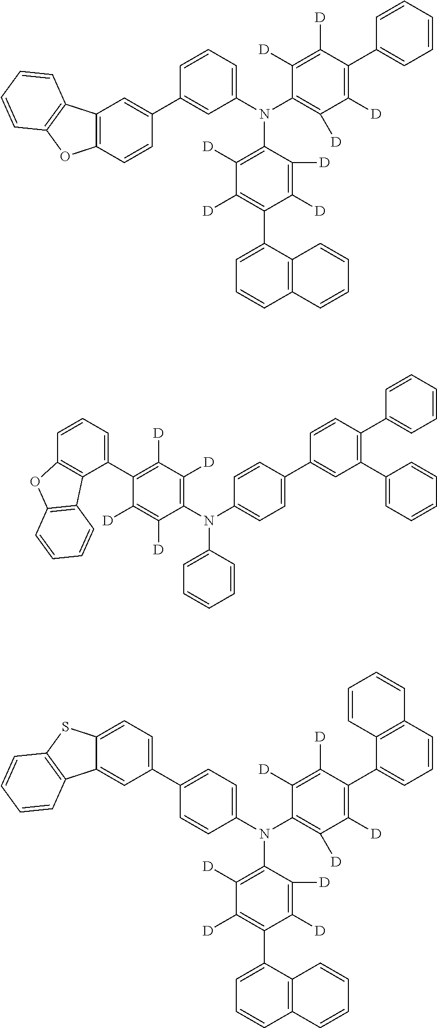 [SMILES: [2H]c1c([2H])c(N(c2cccc(-c3ccc4oc5ccccc5c4c3)c2)c2c([2H])c([2H])c(-c3cccc4ccccc34)c([2H])c2[2H])c([2H])c([2H])c1-c1ccccc1 ; [2H]c1c([2H])c(N(c2ccccc2)c2ccc(-c3ccc(-c4ccccc4)c(-c4ccccc4)c3)cc2)c([2H])c([2H])c1-c1cccc2oc3ccccc3c12 ; [2H]c1c([2H])c(N(c2ccc(-c3ccc4sc5ccccc5c4c3)cc2)c2c([2H])c([2H])c(-c3cccc4ccccc34)c([2H])c2[2H])c([2H])c([2H])c1-c1cccc2ccccc12]

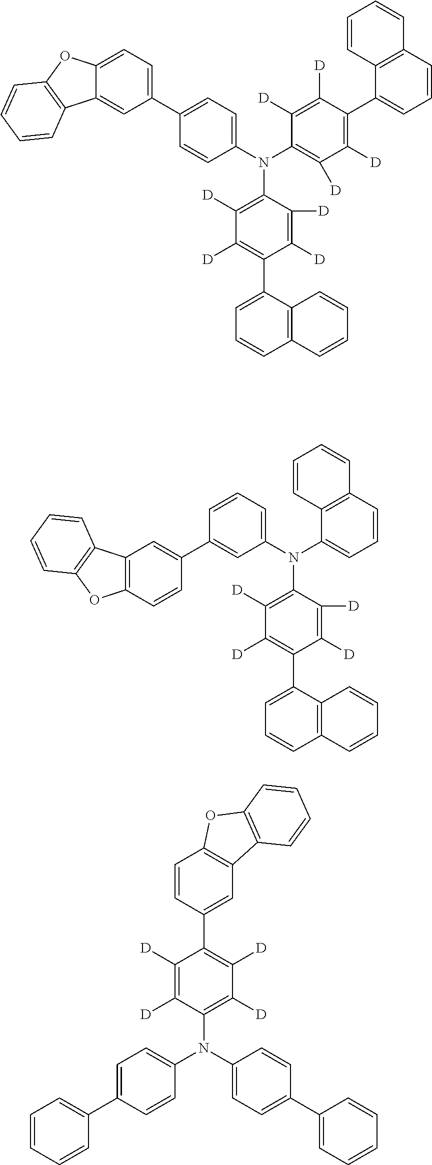

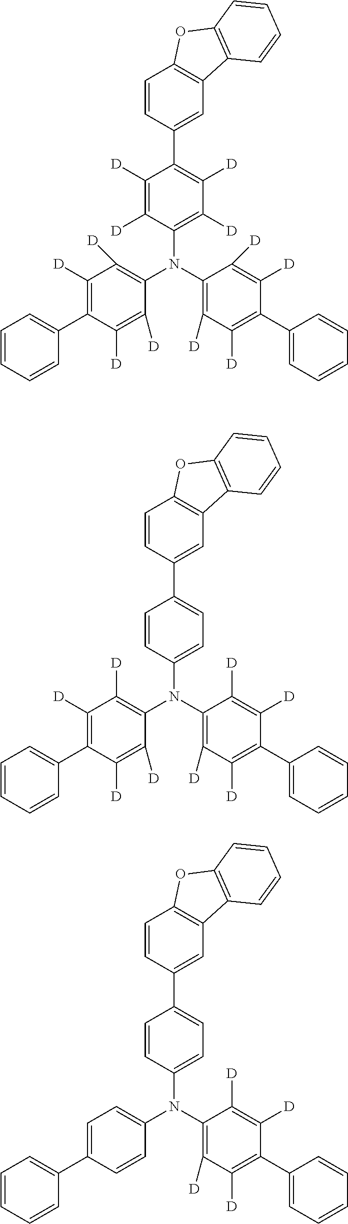 [SMILES: [2H]c1c([2H])c(N(c2c([2H])c([2H])c(-c3ccccc3)c([2H])c2[2H])c2c([2H])c([2H])c(-c3ccc4oc5ccccc5c4c3)c([2H])c2[2H])c([2H])c([2H])c1-c1ccccc1 ; [2H]c1c([2H])c(N(c2ccc(-c3ccc4oc5ccccc5c4c3)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccc4oc5ccccc5c4c3)cc2)c([2H])c([2H])c1-c1ccccc1]

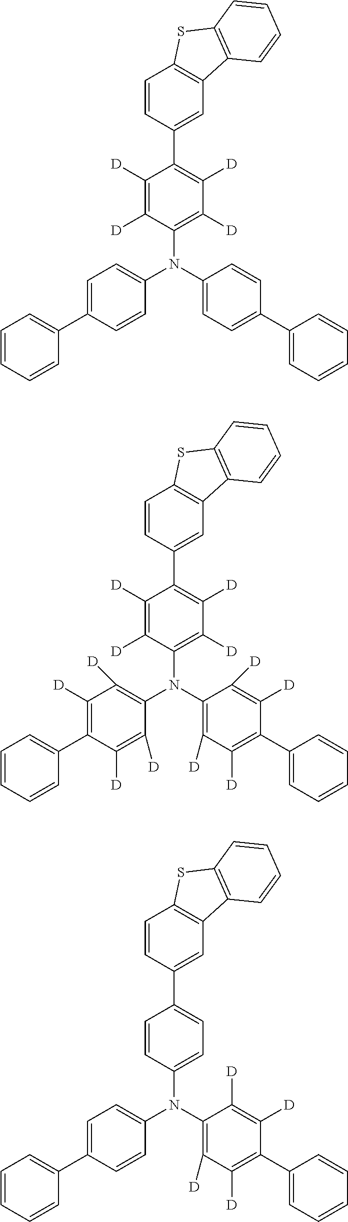 [SMILES: [2H]c1c([2H])c(N(c2ccc(-c3ccccc3)cc2)c2ccc(-c3ccccc3)cc2)c([2H])c([2H])c1-c1ccc2sc3ccccc3c2c1 ; [2H]c1c([2H])c(N(c2c([2H])c([2H])c(-c3ccccc3)c([2H])c2[2H])c2c([2H])c([2H])c(-c3ccc4sc5ccccc5c4c3)c([2H])c2[2H])c([2H])c([2H])c1-c1ccccc1 ; [2H]c1c([2H])c(N(c2ccc(-c3ccccc3)cc2)c2ccc(-c3ccc4sc5ccccc5c4c3)cc2)c([2H])c([2H])c1-c1ccccc1]

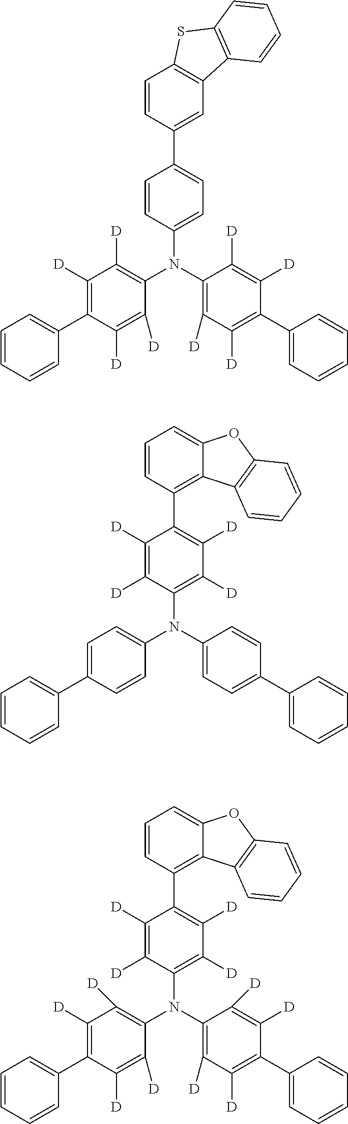 [SMILES: [2H]c1c([2H])c(N(c2ccc(-c3ccc4sc5ccccc5c4c3)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccccc3)cc2)c([2H])c([2H])c1-c1cccc2oc3ccccc3c12 ; [2H]c1c([2H])c(N(c2c([2H])c([2H])c(-c3ccccc3)c([2H])c2[2H])c2c([2H])c([2H])c(-c3cccc4oc5ccccc5c34)c([2H])c2[2H])c([2H])c([2H])c1-c1ccccc1]

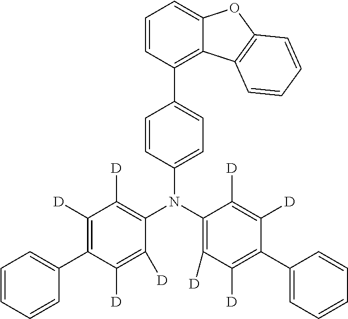 [SMILES: [2H]c1c([2H])c(N(c2ccc(-c3cccc4oc5ccccc5c34)cc2)c2c([2H])c([2H])c(-c3ccccc3)c([2H])c2[2H])c([2H])c([2H])c1-c1ccccc1]

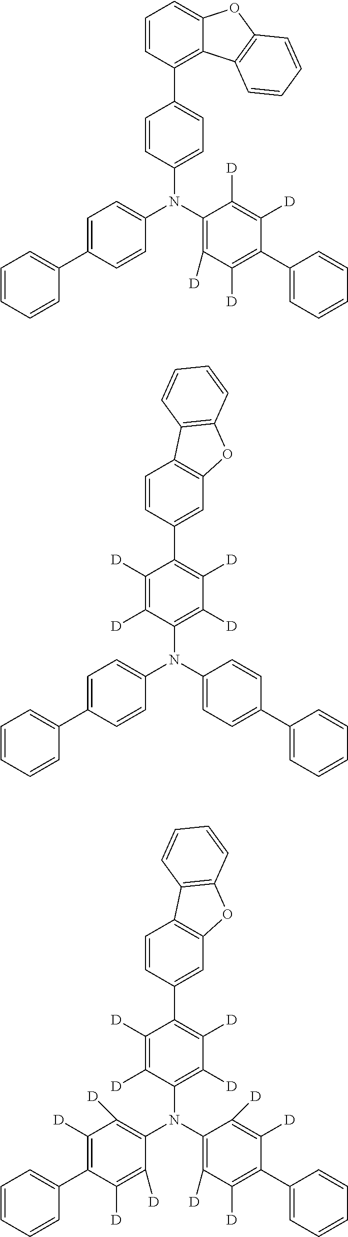 [SMILES: [2H]c1c([2H])c(N(c2ccc(-c3ccccc3)cc2)c2ccc(-c3cccc4oc5ccccc5c34)cc2)c([2H])c([2H])c1-c1ccccc1 ; [2H]c1c([2H])c(N(c2ccc(-c3ccccc3)cc2)c2ccc(-c3ccccc3)cc2)c([2H])c([2H])c1-c1ccc2c(c1)oc1ccccc12 ; [2H]c1c([2H])c(N(c2c([2H])c([2H])c(-c3ccccc3)c([2H])c2[2H])c2c([2H])c([2H])c(-c3ccc4c(c3)oc3ccccc34)c([2H])c2[2H])c([2H])c([2H])c1-c1ccccc1]

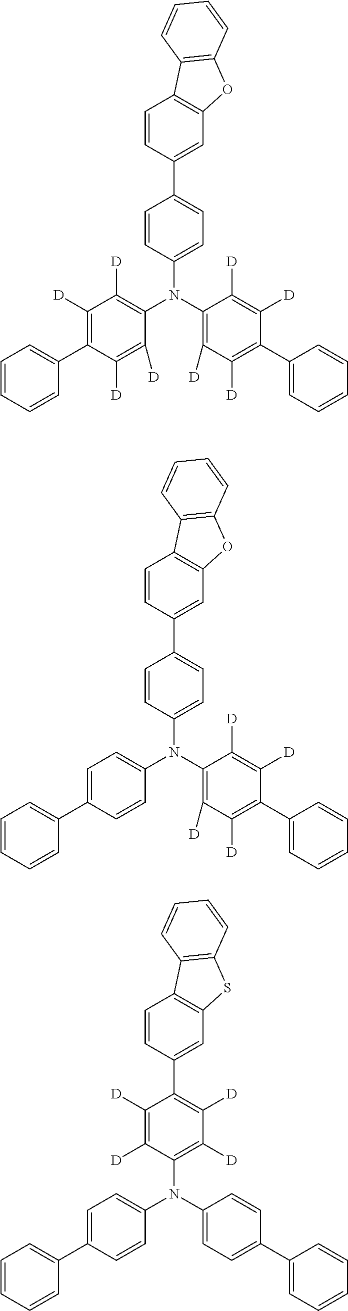 [SMILES: [2H]c1c([2H])c(N(c2ccc(-c3ccc4c(c3)oc3ccccc34)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccc4c(c3)oc3ccccc34)cc2)c([2H])c([2H])c1-c1ccccc1 ; [2H]c1c([2H])c(N(c2ccc(-c3ccccc3)cc2)c2ccc(-c3ccccc3)cc2)c([2H])c([2H])c1-c1ccc2c(c1)sc1ccccc12]

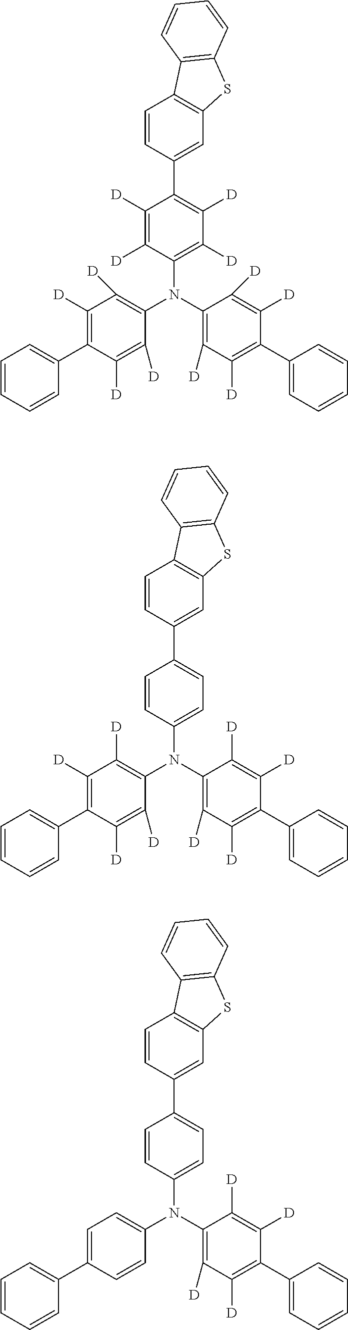 [SMILES: [2H]c1c([2H])c(N(c2c([2H])c([2H])c(-c3ccccc3)c([2H])c2[2H])c2c([2H])c([2H])c(-c3ccc4c(c3)sc3ccccc34)c([2H])c2[2H])c([2H])c([2H])c1-c1ccccc1 ; [2H]c1c([2H])c(N(c2ccc(-c3ccc4c(c3)sc3ccccc34)cc2)c2c([2H])c([2H])c(-c3ccccc3)c([2H])c2[2H])c([2H])c([2H])c1-c1ccccc1 ; [2H]c1c([2H])c(N(c2ccc(-c3ccccc3)cc2)c2ccc(-c3ccc4c(c3)sc3ccccc34)cc2)c([2H])c([2H])c1-c1ccccc1]

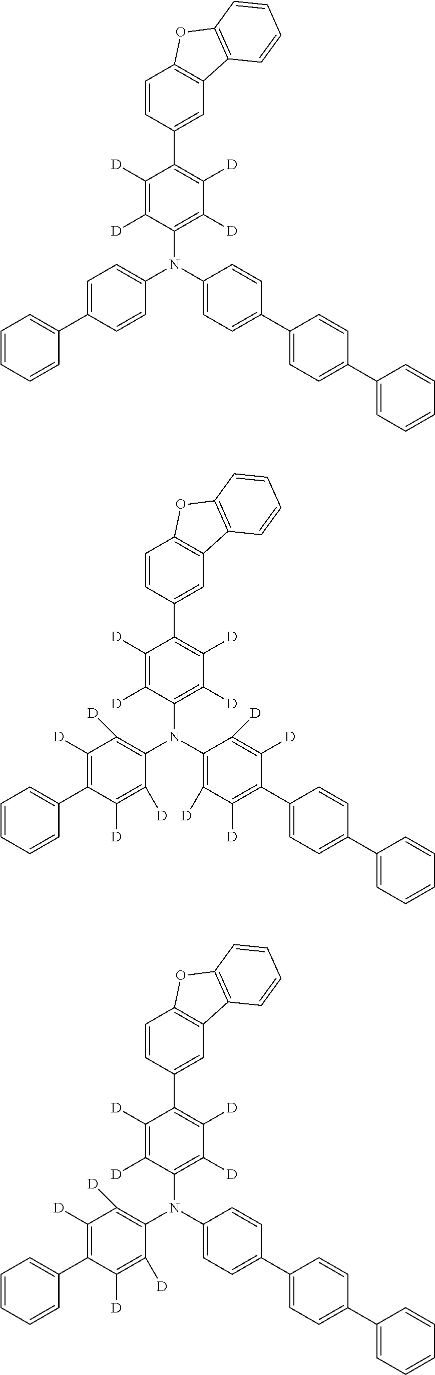 [SMILES: [2H]c1c([2H])c(N(c2ccc(-c3ccccc3)cc2)c2ccc(-c3ccc(-c4ccccc4)cc3)cc2)c([2H])c([2H])c1-c1ccc2oc3ccccc3c2c1 ; [2H]c1c([2H])c(N(c2c([2H])c([2H])c(-c3ccc(-c4ccccc4)cc3)c([2H])c2[2H])c2c([2H])c([2H])c(-c3ccc4oc5ccccc5c4c3)c([2H])c2[2H])c([2H])c([2H])c1-c1ccccc1 ; [2H]c1c([2H])c(N(c2ccc(-c3ccc(-c4ccccc4)cc3)cc2)c2c([2H])c([2H])c(-c3ccc4oc5ccccc5c4c3)c([2H])c2[2H])c([2H])c([2H])c1-c1ccccc1]

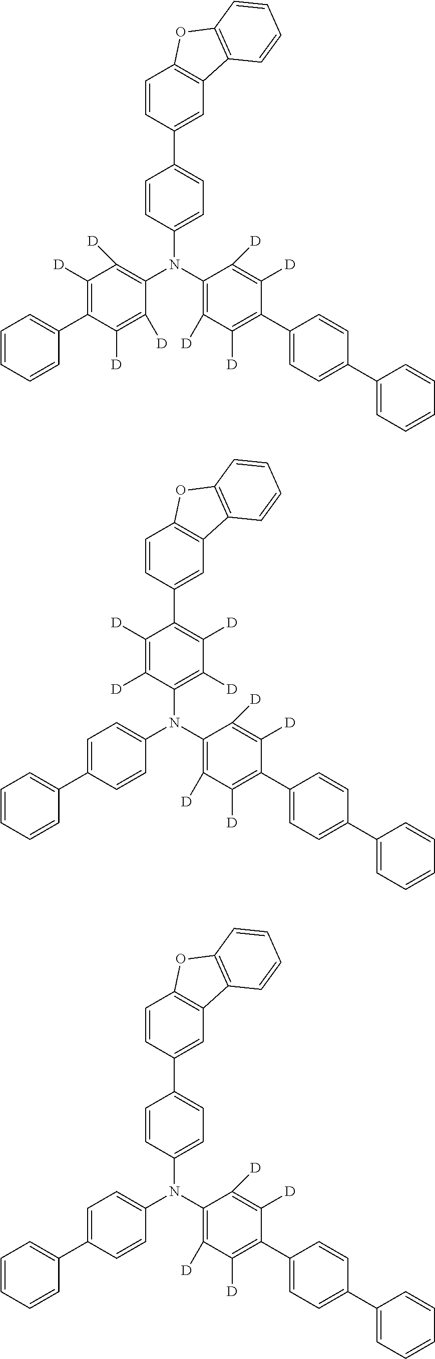 [SMILES: [2H]c1c([2H])c(N(c2ccc(-c3ccc4oc5ccccc5c4c3)cc2)c2c([2H])c([2H])c(-c3ccc(-c4ccccc4)cc3)c([2H])c2[2H])c([2H])c([2H])c1-c1ccccc1 ; [2H]c1c([2H])c(N(c2ccc(-c3ccccc3)cc2)c2c([2H])c([2H])c(-c3ccc4oc5ccccc5c4c3)c([2H])c2[2H])c([2H])c([2H])c1-c1ccc(-c2ccccc2)cc1 ; [2H]c1c([2H])c(N(c2ccc(-c3ccccc3)cc2)c2ccc(-c3ccc4oc5ccccc5c4c3)cc2)c([2H])c([2H])c1-c1ccc(-c2ccccc2)cc1]

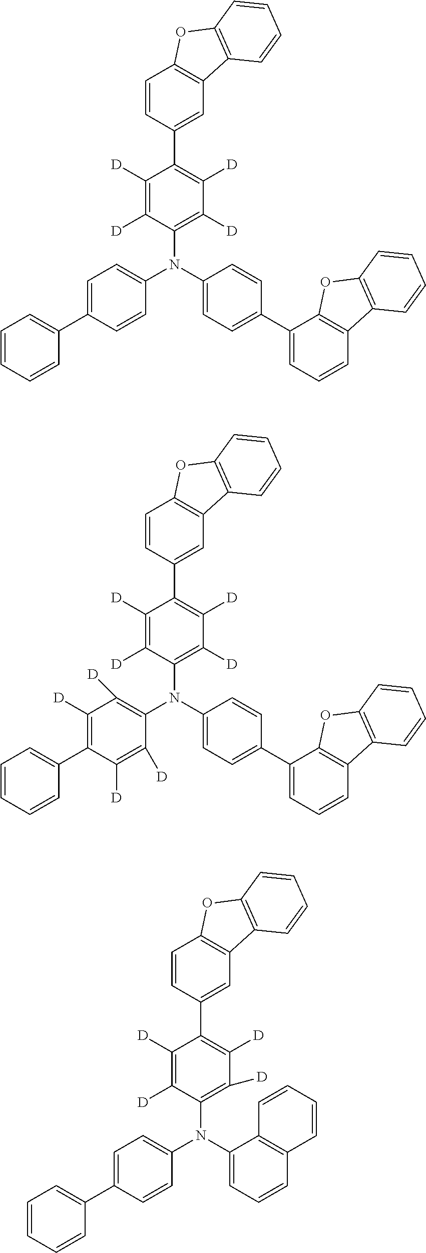 [SMILES: [2H]c1c([2H])c(N(c2ccc(-c3ccccc3)cc2)c2ccc(-c3cccc4c3oc3ccccc34)cc2)c([2H])c([2H])c1-c1ccc2oc3ccccc3c2c1 ; [2H]c1c([2H])c(N(c2ccc(-c3cccc4c3oc3ccccc34)cc2)c2c([2H])c([2H])c(-c3ccc4oc5ccccc5c4c3)c([2H])c2[2H])c([2H])c([2H])c1-c1ccccc1 ; [2H]c1c([2H])c(N(c2ccc(-c3ccccc3)cc2)c2cccc3ccccc23)c([2H])c([2H])c1-c1ccc2oc3ccccc3c2c1]

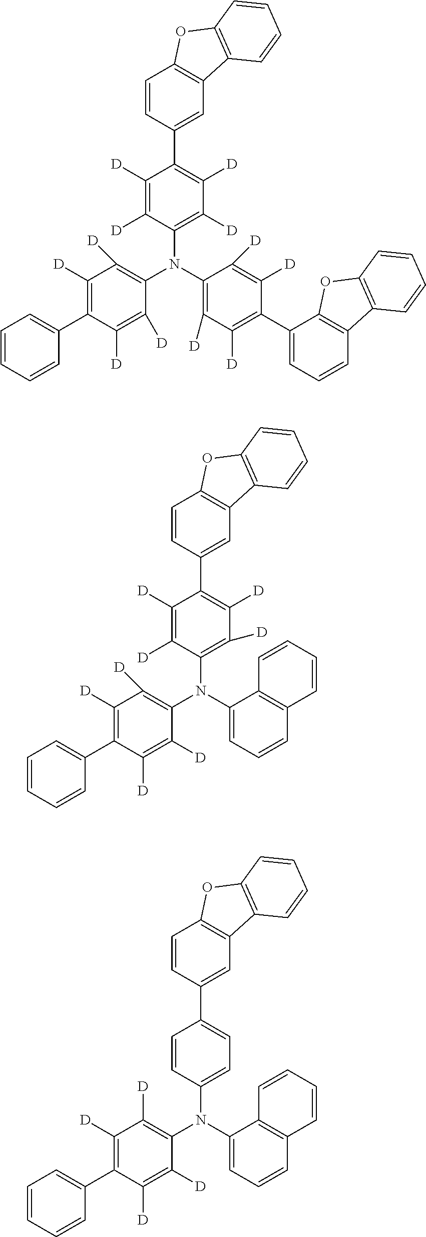 [SMILES: [2H]c1c([2H])c(N(c2c([2H])c([2H])c(-c3ccc4oc5ccccc5c4c3)c([2H])c2[2H])c2c([2H])c([2H])c(-c3cccc4c3oc3ccccc34)c([2H])c2[2H])c([2H])c([2H])c1-c1ccccc1 ; [2H]c1c([2H])c(N(c2c([2H])c([2H])c(-c3ccc4oc5ccccc5c4c3)c([2H])c2[2H])c2cccc3ccccc23)c([2H])c([2H])c1-c1ccccc1 ; [2H]c1c([2H])c(N(c2ccc(-c3ccc4oc5ccccc5c4c3)cc2)c2cccc3ccccc23)c([2H])c([2H])c1-c1ccccc1]

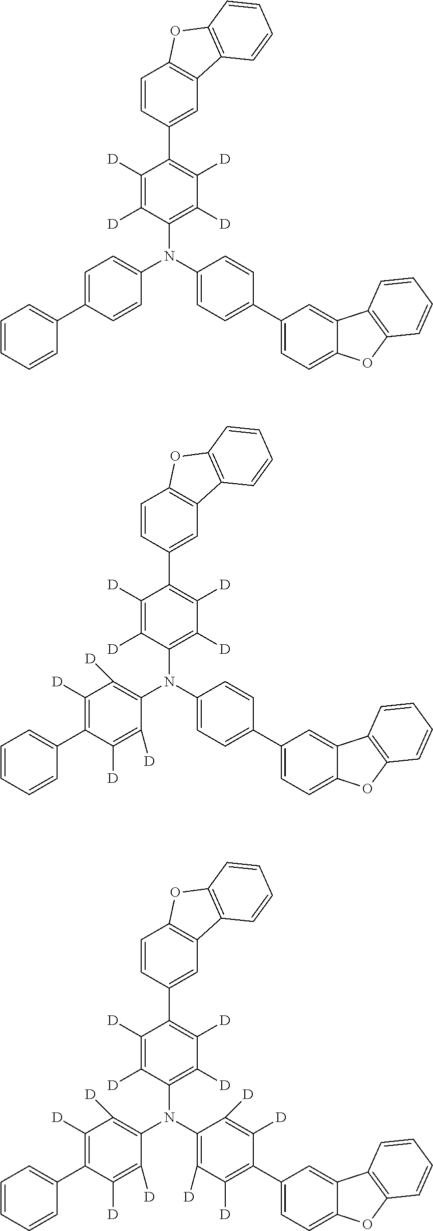 [SMILES: [2H]c1c([2H])c(N(c2ccc(-c3ccccc3)cc2)c2ccc(-c3ccc4oc5ccccc5c4c3)cc2)c([2H])c([2H])c1-c1ccc2oc3ccccc3c2c1 ; [2H]c1c([2H])c(N(c2ccc(-c3ccc4oc5ccccc5c4c3)cc2)c2c([2H])c([2H])c(-c3ccc4oc5ccccc5c4c3)c([2H])c2[2H])c([2H])c([2H])c1-c1ccccc1 ; [2H]c1c([2H])c(N(c2c([2H])c([2H])c(-c3ccc4oc5ccccc5c4c3)c([2H])c2[2H])c2c([2H])c([2H])c(-c3ccc4oc5ccccc5c4c3)c([2H])c2[2H])c([2H])c([2H])c1-c1ccccc1]

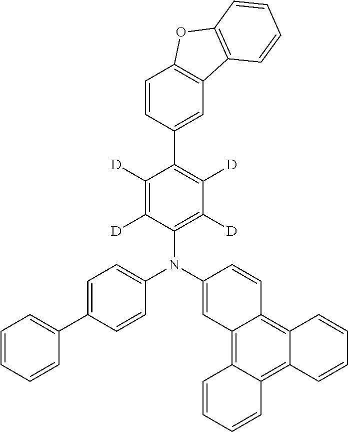 [SMILES: [2H]c1c([2H])c(N(c2ccc(-c3ccccc3)cc2)c2ccc3c4ccccc4c4ccccc4c3c2)c([2H])c([2H])c1-c1ccc2oc3ccccc3c2c1]

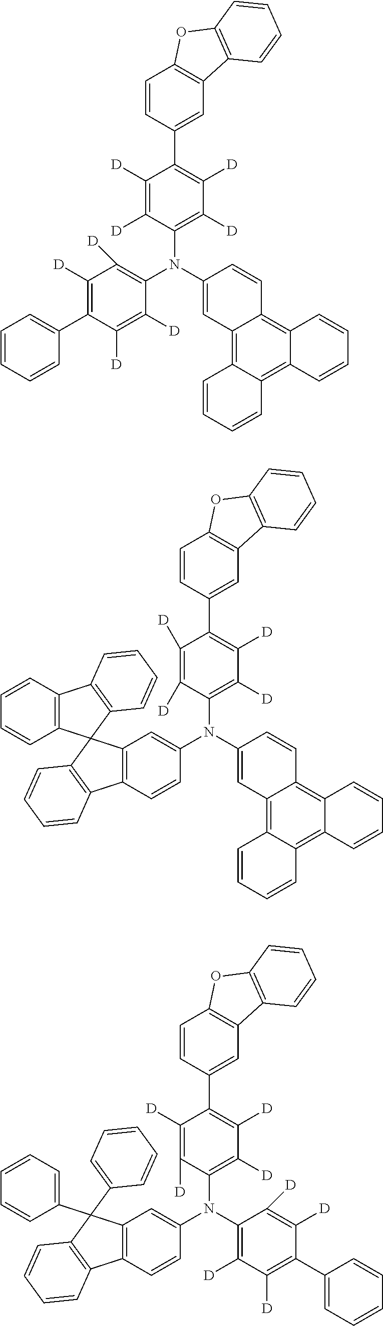 [SMILES: [2H]c1c([2H])c(N(c2ccc3c4ccccc4c4ccccc4c3c2)c2c([2H])c([2H])c(-c3ccc4oc5ccccc5c4c3)c([2H])c2[2H])c([2H])c([2H])c1-c1ccccc1 ; [2H]c1c([2H])c(N(c2ccc3c(c2)C2(c4ccccc4-c4ccccc42)c2ccccc2-3)c2ccc3c4ccccc4c4ccccc4c3c2)c([2H])c([2H])c1-c1ccc2oc3ccccc3c2c1 ; [2H]c1c([2H])c(N(c2ccc3c(c2)C(c2ccccc2)(c2ccccc2)c2ccccc2-3)c2c([2H])c([2H])c(-c3ccc4oc5ccccc5c4c3)c([2H])c2[2H])c([2H])c([2H])c1-c1ccccc1]

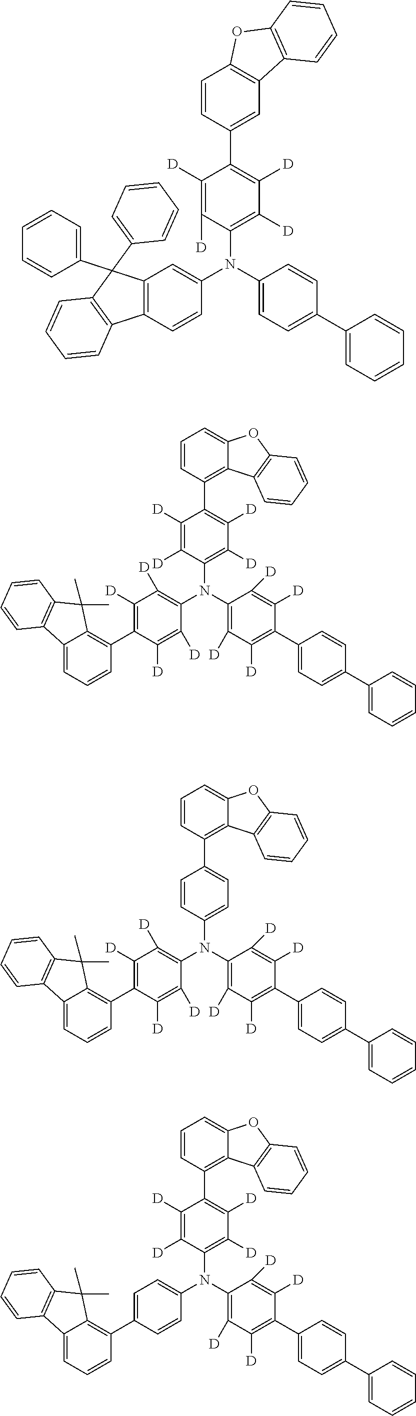

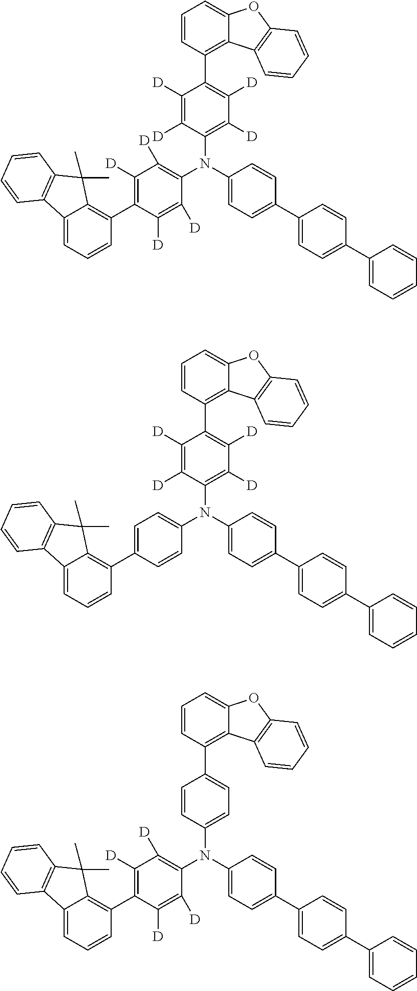 [SMILES: [2H]c1c([2H])c(N(c2ccc(-c3ccc(-c4ccccc4)cc3)cc2)c2c([2H])c([2H])c(-c3cccc4oc5ccccc5c34)c([2H])c2[2H])c([2H])c([2H])c1-c1cccc2c1C(C)(C)c1ccccc1-2 ; [2H]c1c([2H])c(N(c2ccc(-c3ccc(-c4ccccc4)cc3)cc2)c2ccc(-c3cccc4c3C(C)(C)c3ccccc3-4)cc2)c([2H])c([2H])c1-c1cccc2oc3ccccc3c12 ; [2H]c1c([2H])c(N(c2ccc(-c3ccc(-c4ccccc4)cc3)cc2)c2ccc(-c3cccc4oc5ccccc5c34)cc2)c([2H])c([2H])c1-c1cccc2c1C(C)(C)c1ccccc1-2]

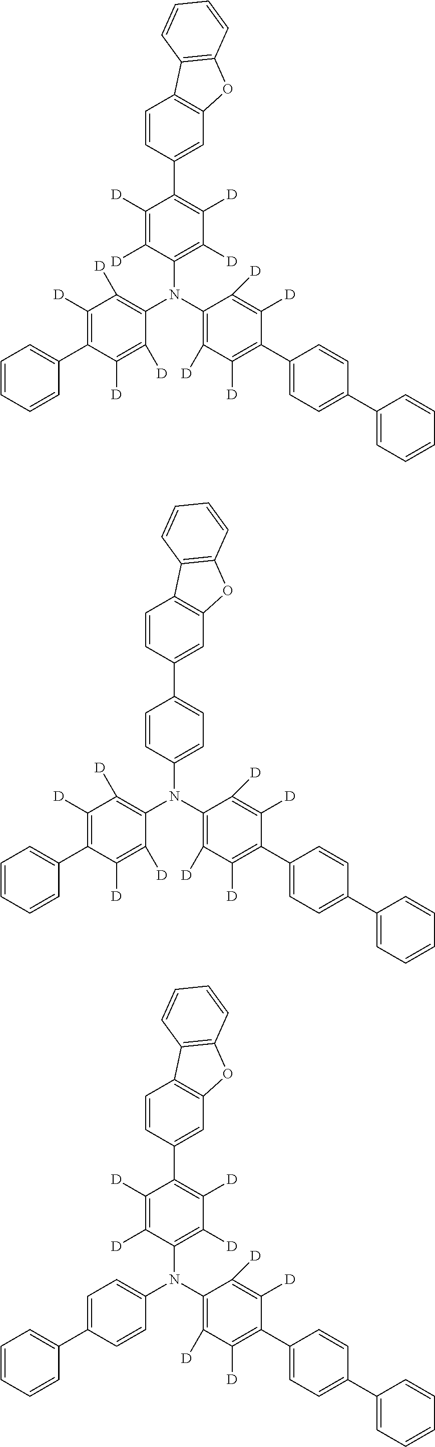 [SMILES: [2H]c1c([2H])c(N(c2c([2H])c([2H])c(-c3ccc(-c4ccccc4)cc3)c([2H])c2[2H])c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccc4c(c3)oc3ccccc34)cc2)c2c([2H])c([2H])c(-c3ccc(-c4ccccc4)cc3)c([2H])c2[2H])c([2H])c([2H])c1-c1ccccc1 ; [2H]c1c([2H])c(N(c2ccc(-c3ccccc3)cc2)c2c([2H])c([2H])c(-c3ccc4c(c3)oc3ccccc34)c([2H])c2[2H])c([2H])c([2H])c1-c1ccc(-c2ccccc2)cc1]

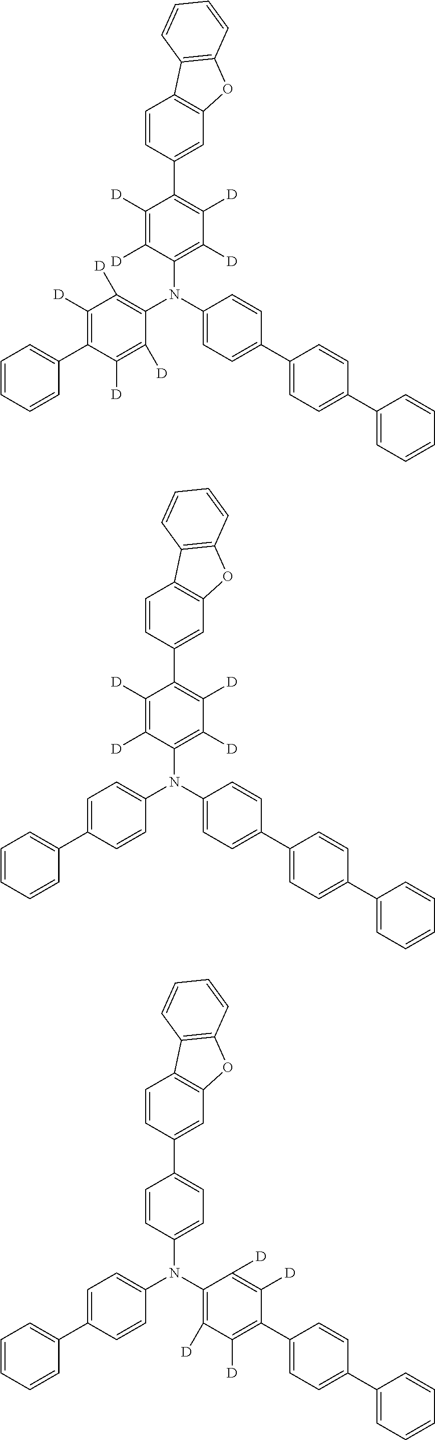 [SMILES: [2H]c1c([2H])c(N(c2ccc(-c3ccc(-c4ccccc4)cc3)cc2)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccccc3)cc2)c2ccc(-c3ccc(-c4ccccc4)cc3)cc2)c([2H])c([2H])c1-c1ccc2c(c1)oc1ccccc12 ; [2H]c1c([2H])c(N(c2ccc(-c3ccccc3)cc2)c2ccc(-c3ccc4c(c3)oc3ccccc34)cc2)c([2H])c([2H])c1-c1ccc(-c2ccccc2)cc1]

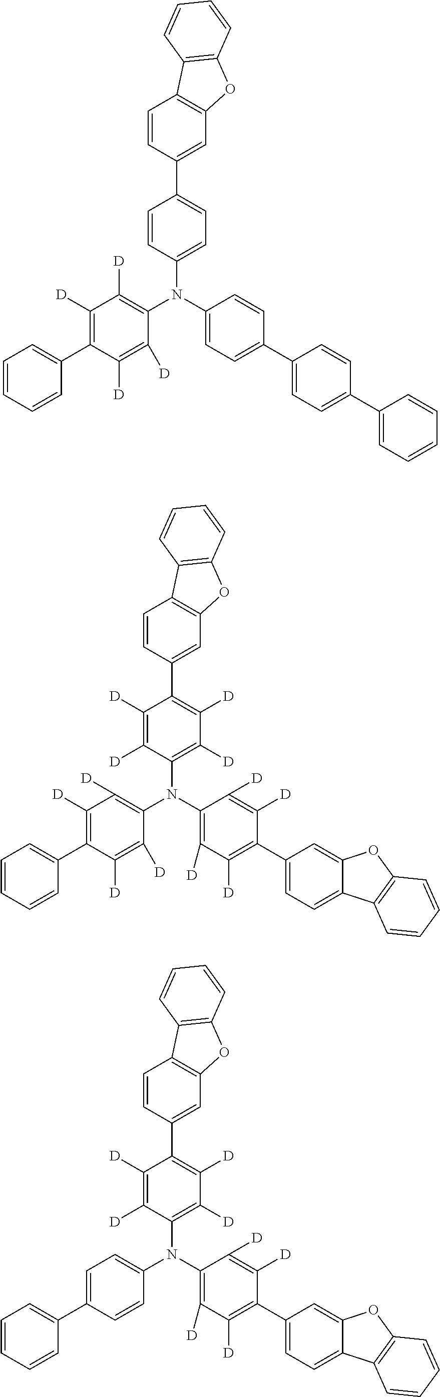 [SMILES: [2H]c1c([2H])c(N(c2ccc(-c3ccc(-c4ccccc4)cc3)cc2)c2ccc(-c3ccc4c(c3)oc3ccccc34)cc2)c([2H])c([2H])c1-c1ccccc1 ; [2H]c1c([2H])c(N(c2c([2H])c([2H])c(-c3ccc4c(c3)oc3ccccc34)c([2H])c2[2H])c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccccc3)cc2)c2c([2H])c([2H])c(-c3ccc4c(c3)oc3ccccc34)c([2H])c2[2H])c([2H])c([2H])c1-c1ccc2c(c1)oc1ccccc12]

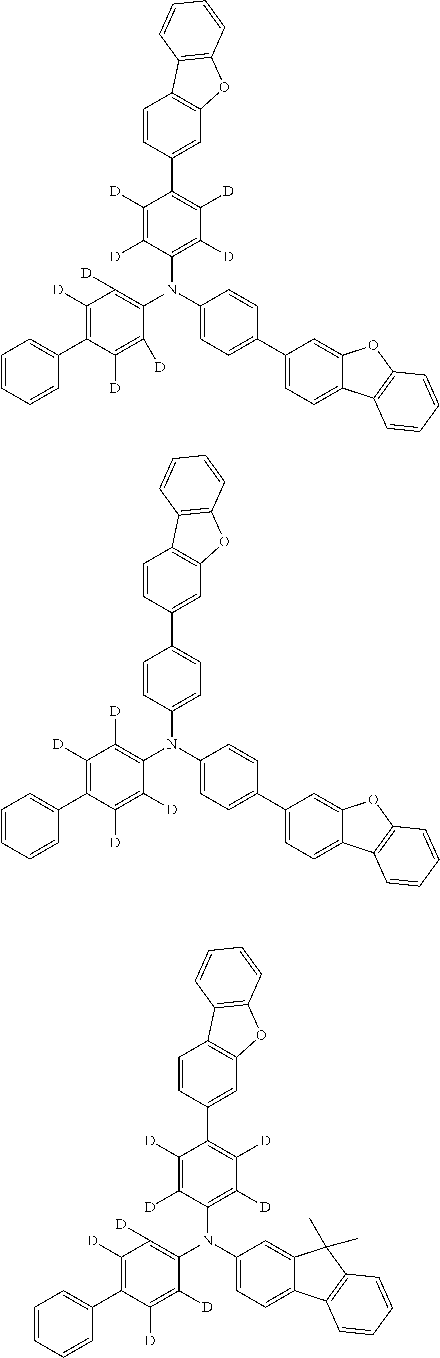 [SMILES: [2H]c1c([2H])c(N(c2ccc(-c3ccc4c(c3)oc3ccccc34)cc2)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccc4c(c3)oc3ccccc34)cc2)c2ccc(-c3ccc4c(c3)oc3ccccc34)cc2)c([2H])c([2H])c1-c1ccccc1 ; [2H]c1c([2H])c(N(c2ccc3c(c2)C(C)(C)c2ccccc2-3)c2c([2H])c([2H])c(-c3ccc4c(c3)oc3ccccc34)c([2H])c2[2H])c([2H])c([2H])c1-c1ccccc1]

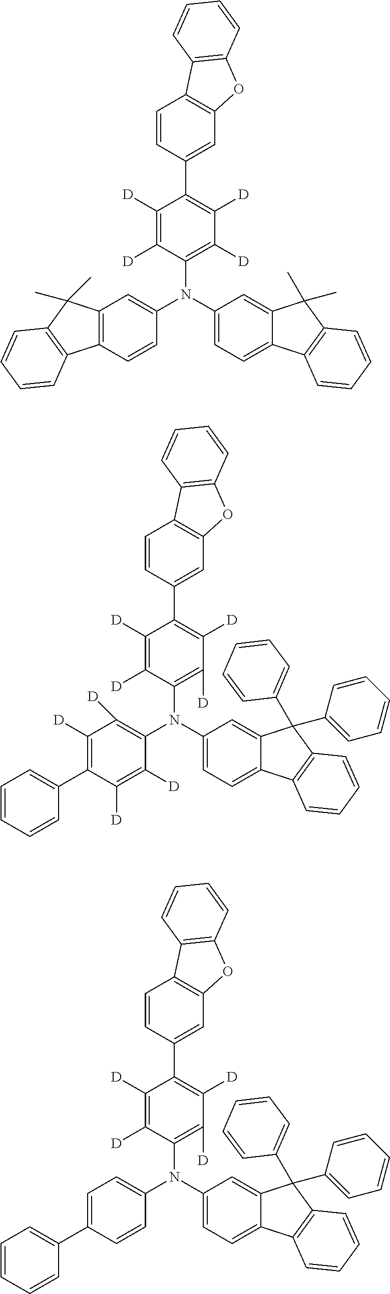 [SMILES: [2H]c1c([2H])c(N(c2ccc3c(c2)C(C)(C)c2ccccc2-3)c2ccc3c(c2)C(C)(C)c2ccccc2-3)c([2H])c([2H])c1-c1ccc2c(c1)oc1ccccc12 ; [2H]c1c([2H])c(N(c2ccc3c(c2)C(c2ccccc2)(c2ccccc2)c2ccccc2-3)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc(-c3ccccc3)cc2)c2ccc3c(c2)C(c2ccccc2)(c2ccccc2)c2ccccc2-3)c([2H])c([2H])c1-c1ccc2c(c1)oc1ccccc12]

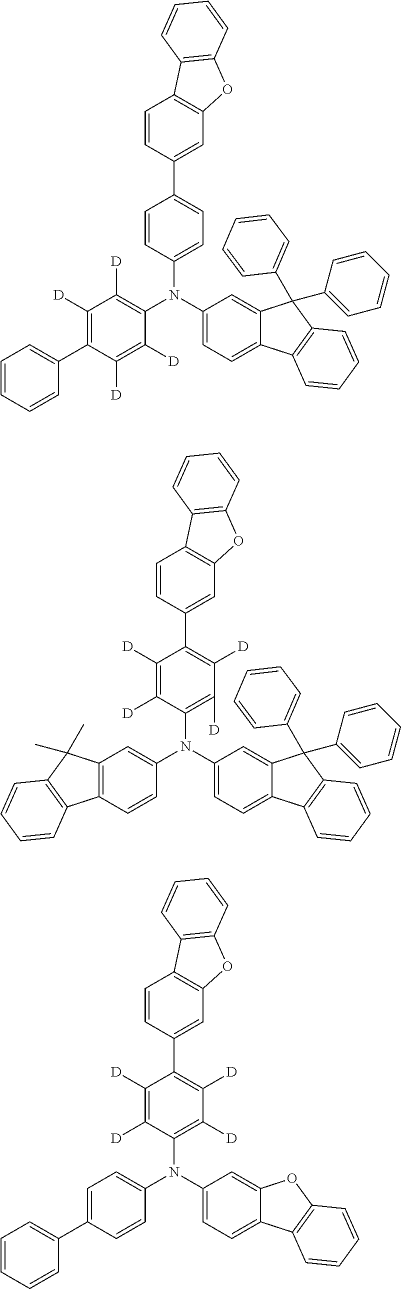 [SMILES: [2H]c1c([2H])c(N(c2ccc(-c3ccc4c(c3)oc3ccccc34)cc2)c2ccc3c(c2)C(c2ccccc2)(c2ccccc2)c2ccccc2-3)c([2H])c([2H])c1-c1ccccc1 ; [2H]c1c([2H])c(N(c2ccc3c(c2)C(C)(C)c2ccccc2-3)c2ccc3c(c2)C(c2ccccc2)(c2ccccc2)c2ccccc2-3)c([2H])c([2H])c1-c1ccc2c(c1)oc1ccccc12 ; [2H]c1c([2H])c(N(c2ccc(-c3ccccc3)cc2)c2ccc3c(c2)oc2ccccc23)c([2H])c([2H])c1-c1ccc2c(c1)oc1ccccc12]

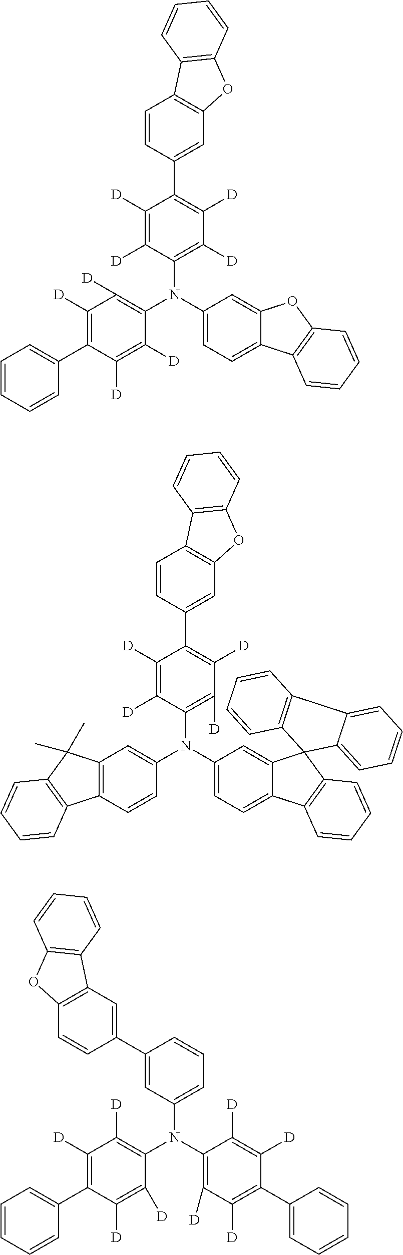 [SMILES: [2H]c1c([2H])c(N(c2ccc3c(c2)oc2ccccc23)c2c([2H])c([2H])c(-c3ccc4c(c3)oc3ccccc34)c([2H])c2[2H])c([2H])c([2H])c1-c1ccccc1 ; [2H]c1c([2H])c(N(c2ccc3c(c2)C(C)(C)c2ccccc2-3)c2ccc3c(c2)C2(c4ccccc4-c4ccccc42)c2ccccc2-3)c([2H])c([2H])c1-c1ccc2c(c1)oc1ccccc12 ; [2H]c1c([2H])c(N(c2cccc(-c3ccc4oc5ccccc5c4c3)c2)c2c([2H])c([2H])c(-c3ccccc3)c([2H])c2[2H])c([2H])c([2H])c1-c1ccccc1]

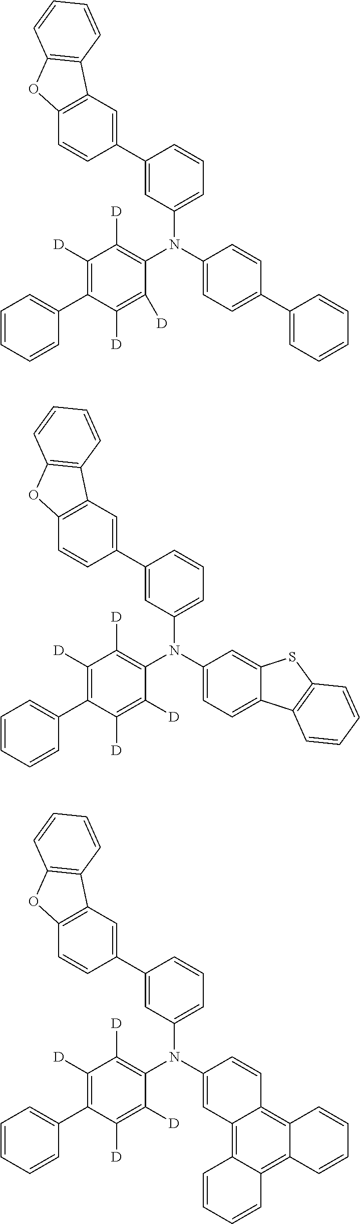 [SMILES: [2H]c1c([2H])c(N(c2ccc(-c3ccccc3)cc2)c2cccc(-c3ccc4oc5ccccc5c4c3)c2)c([2H])c([2H])c1-c1ccccc1 ; [2H]c1c([2H])c(N(c2cccc(-c3ccc4oc5ccccc5c4c3)c2)c2ccc3c(c2)sc2ccccc23)c([2H])c([2H])c1-c1ccccc1 ; [2H]c1c([2H])c(N(c2cccc(-c3ccc4oc5ccccc5c4c3)c2)c2ccc3c4ccccc4c4ccccc4c3c2)c([2H])c([2H])c1-c1ccccc1]

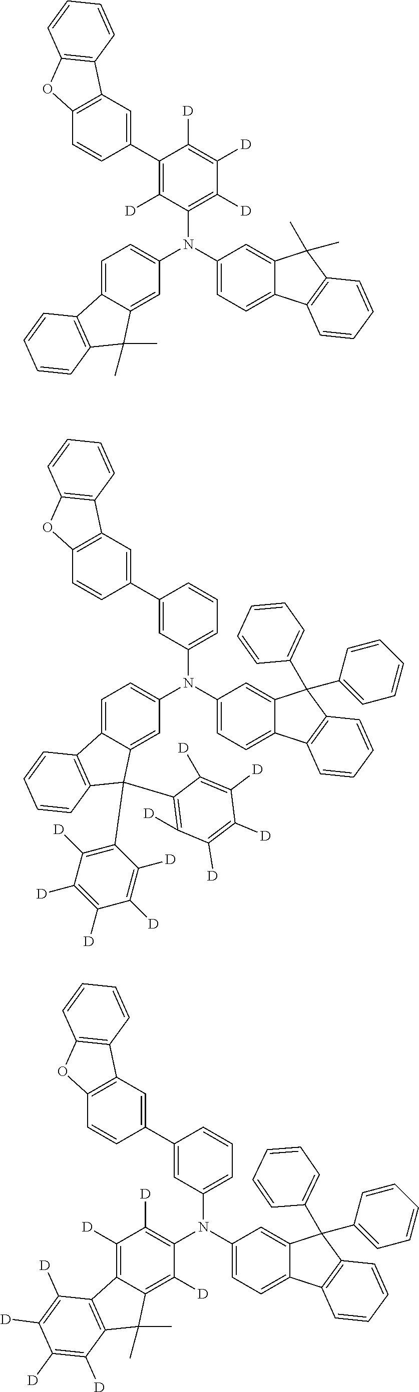

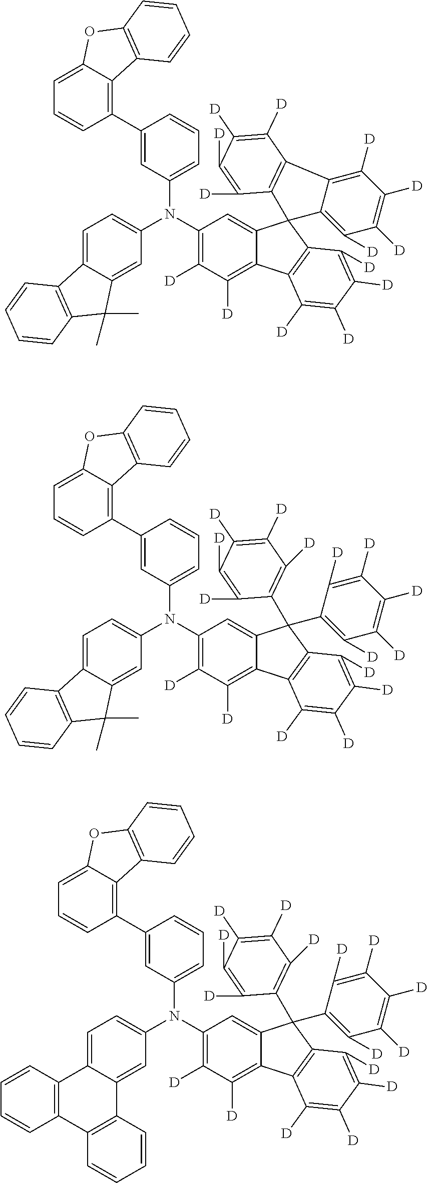

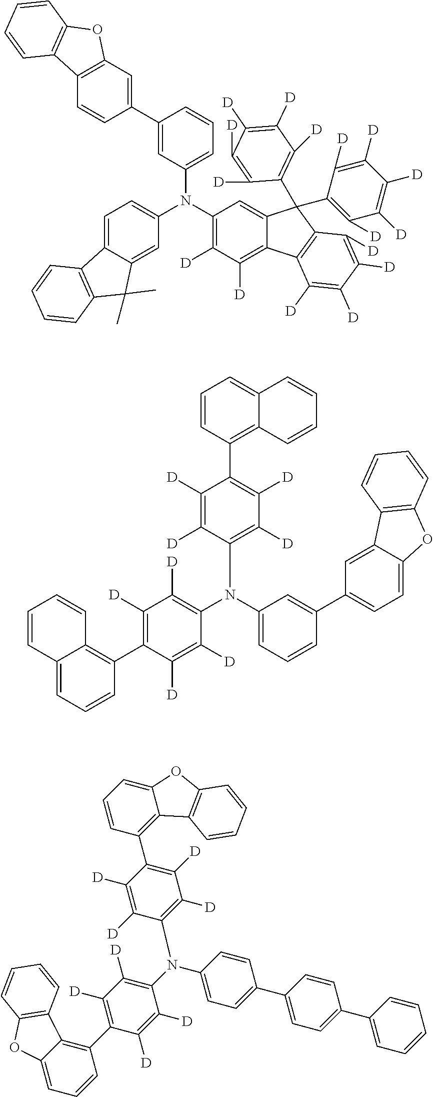 [SMILES: [2H]c1c(N(c2cccc(-c3ccc4c(c3)oc3ccccc34)c2)c2ccc3c(c2)C(C)(C)c2ccccc2-3)cc2c(c1[2H])-c1c([2H])c([2H])c([2H])c([2H])c1C2(c1c([2H])c([2H])c([2H])c([2H])c1[2H])c1c([2H])c([2H])c([2H])c([2H])c1[2H] ; [2H]c1c([2H])c(N(c2cccc(-c3ccc4oc5ccccc5c4c3)c2)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccc(-c4ccccc4)cc3)cc2)c2c([2H])c([2H])c(-c3cccc4oc5ccccc5c34)c([2H])c2[2H])c([2H])c([2H])c1-c1cccc2oc3ccccc3c12]

### Material for Organic EL Devices

The material for organic EL devices which is an aspect of the present invention contains the inventive compound (1). The content of the inventive compound (1) in the material for organic EL devices is 1% by mass or more (including 100%), preferably 10% by mass or more (including 100%), more preferably 50% by mass or more (including 100%), further preferably 80% by mass or more (including 100%), and particularly preferably 90% by mass or more (including 100%). The material for organic EL devices which is an aspect of the present invention is useful for production of organic EL devices.

In an aspect of the present invention, the inventive compound (1) is preferably a material for a hole transporting layer.

In an aspect of the present invention, the material for organic EL devices preferably further contains a protium form of the inventive compound (1). The protium form is an inventive compound in which all the hydrogen atoms are each a protium atom.

The molar ratio of the inventive compound (1) and the protium form of the inventive compound (1) mixed (inventive compound:protium form) is preferably 10:90 to 90:10, more preferably 20:80 to 80:20, further preferably 30:70 to 70:30, and particularly preferably 40:60 to 60:40.

A material for organic electroluminescent devices according to an aspect of the present invention is a material for a hole transporting layer.

The content of the inventive compound (1) in the material for organic electroluminescent devices is preferably 1% by mass or more (including 100%), more preferably 10% by mass or more (including 100%), further preferably 50% by mass or more (including 100%), furthermore preferably 80% by mass or more (including 100%), and particularly preferably 90% by mass or more (including 100%).

### Organic EL Device

The organic EL device which is an aspect of the present invention includes an anode, a cathode, and organic layers disposed between the anode and the cathode. The organic layers include a light emitting layer, and at least one layer of the organic layers contains the inventive compound.

Examples of the organic layer containing the inventive compound include a hole transporting zone (a hole injecting layer, a hole transporting layer, an electron blocking layer, an exciton blocking layer, etc.) provided between the anode and the light emitting layer, a light emitting layer, a space layer, and an electron transporting zone (an electron injecting layer, an electron transporting layer, a hole blocking layer, etc.) provided between the cathode and the light emitting layer, but are not limited thereto. The inventive compound is preferably used as a material for the hole transporting zone or the light emitting layer of a fluorescent or phosphorescent EL device, more preferably as a material for the hole transporting zone, further preferably as a material for the hole injecting layer, the hole transporting layer, the electron blocking layer, or the exciton blocking layer, and particularly preferably as a material for the hole injecting layer or the hole transporting layer.

The organic EL device which is an aspect of the present invention may be a fluorescent or phosphorescent light emission-type monochromatic light emitting device or a fluorescent/phosphorescent hybrid-type white light emitting device, and may be a simple type having a single light emitting unit or a tandem type having a plurality of light emitting units. Above all, a fluorescent light emission-type device is preferred. The “light emitting unit” herein refers to a minimum unit that emits light through recombination of injected holes and electrons, which includes organic layers among which at least one layer is a light emitting layer.

For example, as a typical device configuration of the simple-type organic EL device, the following device configurations can be exemplified.

### (1) Anode/Light Emitting Unit/Cathode

The light emitting unit may be a multilayer type having a plurality of phosphorescent light emitting layers or fluorescent light emitting layers. In this case, a space layer may be provided between the light emitting layers for the purpose of preventing excitons generated in the phosphorescent light emitting layer from diffusing into the fluorescent light emitting layer. Typical layer configurations of the simple type light emitting unit are described below. Layers in parentheses are optional.
(a) (hole injecting layer/) hole transporting layer/fluorescent light emitting layer/electron transporting layer (/electron injecting layer)
(b) (hole injecting layer/) hole transporting layer/phosphorescent light emitting layer/electron transporting layer (/electron injecting layer)
(c) (hole injecting layer/) hole transporting layer/first fluorescent light emitting layer/second fluorescent light emitting layer/electron transporting layer (/electron injecting layer)
(d) (hole injecting layer/) hole transporting layer/first phosphorescent light emitting layer/second phosphorescent light emitting layer/electron transporting layer (/electron injecting layer)
(e) (hole injecting layer/) hole transporting layer/phosphorescent light emitting layer/space layer/fluorescent light emitting layer/electron transporting layer (/electron injecting layer)
(f) (hole injecting layer/) hole transporting layer/first phosphorescent light emitting layer/second phosphorescent light emitting layer/space layer/fluorescent light emitting layer/electron transporting layer (/electron injecting layer)
(g) (hole injecting layer/) hole transporting layer/first phosphorescent light emitting layer/space layer/second phosphorescent light emitting layer/space layer/fluorescent light emitting layer/electron transporting layer (/electron injecting layer)
(h) (hole injecting layer/) hole transporting layer/phosphorescent light emitting layer/space layer/first fluorescent light emitting layer/second fluorescent light emitting layer/electron transporting layer (/electron injecting layer)
(i) (hole injecting layer/) hole transporting layer/electron blocking layer/fluorescent light emitting layer/electron transporting layer (/electron injecting layer)
(j) (hole injecting layer/) hole transporting layer/electron blocking layer/phosphorescent light emitting layer/electron transporting layer (/electron injecting layer)
(k) (hole injecting layer/) hole transporting layer/exciton blocking layer/fluorescent light emitting layer/electron transporting layer (/electron injecting layer)
(l) (hole injecting layer/) hole transporting layer/exciton blocking layer/phosphorescent light emitting layer/electron transporting layer (/electron injecting layer)
(m) (hole injecting layer/) first hole transporting layer/second hole transporting layer/fluorescent light emitting layer/electron transporting layer (/electron injecting layer)
(m1) (hole injecting layer/) first hole transporting layer/second hole transporting layer/third hole transporting layer/fluorescent light emitting layer/electron transporting layer (/electron injecting layer)
(n) (hole injecting layer/) first hole transporting layer/second hole transporting layer/phosphorescent light emitting layer/electron transporting layer (/electron injecting layer)
(n1) (hole injecting layer/) first hole transporting layer/second hole transporting layer/third hole transporting layer/phosphorescent light emitting layer/electron transporting layer (/electron injecting layer)
(o) (hole injecting layer/) first hole transporting layer/second hole transporting layer/fluorescent light emitting layer/first electron transporting layer/second electron transporting layer (/electron injecting layer)
(o1) (hole injecting layer/) first hole transporting layer/second hole transporting layer/third hole transporting layer/fluorescent light emitting layer/first electron transporting layer/second electron transporting layer (/electron injecting layer)
(p) (hole injecting layer/) first hole transporting layer/second hole transporting layer/phosphorescent light emitting layer/first electron transporting layer/second electron transporting layer (/electron injecting layer)
(p1) (hole injecting layer/) first hole transporting layer/second hole transporting layer/third hole transporting layer/phosphorescent light emitting layer/first electron transporting layer/second electron transporting layer (/electron injecting layer)
(q) (hole injecting layer/) hole transporting layer/fluorescent light emitting layer/hole blocking layer/electron transporting layer (/electron injecting layer)
(r) (hole injecting layer/) hole transporting layer/phosphorescent light emitting layer/hole blocking layer/electron transporting layer (/electron injecting layer)
(s) (hole injecting layer/) hole transporting layer/fluorescent light emitting layer/exciton blocking layer/electron transporting layer (/electron injecting layer)
(t) (hole injecting layer/) hole transporting layer/phosphorescent light emitting layer/exciton blocking layer/electron transporting layer (/electron injecting layer)

The phosphorescent and fluorescent light emitting layers may exhibit emission colors different from each other. Specifically, in the light emitting unit (f), a layer configuration, such as (hole injecting layer/) hole transporting layer/first phosphorescent light emitting layer (red color emission)/second phosphorescent light emitting layer (green color emission)/space layer/fluorescent light emitting layer (blue color emission)/electron transporting layer, may be exemplified.

An electron blocking layer may be properly provided between each light emitting layer and the hole transporting layer or the space layer. A hole blocking layer may be properly provided between each light emitting layer and the electron transporting layer. The employment of the electron blocking layer or the hole blocking layer allows for improvement of the emission efficiency by confining electrons or holes within the light emitting layer to increase the probability of charge recombination in the light emitting layer.

When the hole transporting layer has a multilayer structure including two or more hole transporting layers, in the multilayer structure, the hole transporting layer adjacent to the light emitting layer, such as the second hole transporting layer of the two-layer structure or the third hole transporting layer of the three-layer structure, may have a function as the electron blocking layer. That is, when the hole transporting layer has a multilayer structure containing two or more hole transporting layers, in the multilayer structure, the hole transporting layer adjacent to the light emitting layer can be used as the electron blocking layer

As a typical device configuration of the tandem-type organic EL device, the following device configurations can be exemplified.

### (2) Anode/First Light Emitting Unit/Intermediate Layer/Second Light Emitting Unit/Cathode

The first light emitting unit and the second light emitting unit herein can be each independently selected, for example, from the above-described light emitting units. The intermediate layer also generally referred to as an intermediate electrode, an intermediate conductive layer, a charge generation layer, an electron withdrawing layer, a connecting layer, or an intermediate insulating layer, and a known material configuration in which electrons are supplied to the first light emitting unit and holes are supplied to the second light emitting unit can be used.

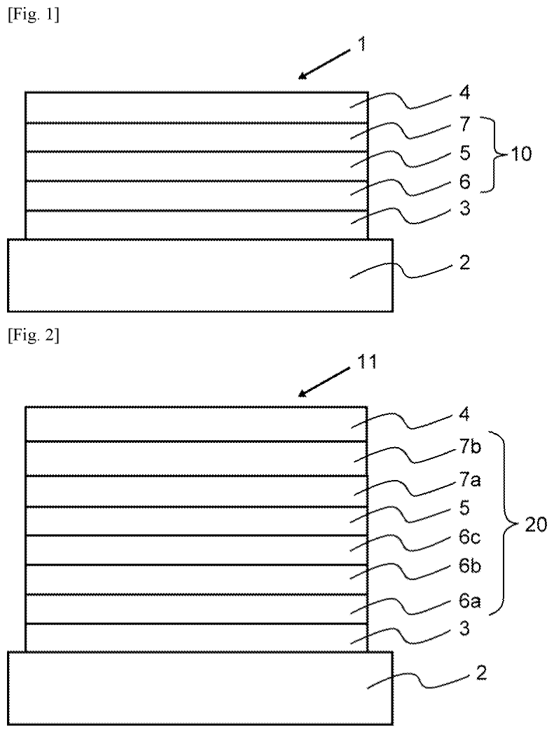

FIG. 1 is a schematic illustration showing an example of the configuration of the organic EL device according to an aspect of the present invention. An organic EL device 1 includes a substrate 2, an anode 3, a cathode 4, and a light emitting unit 10 disposed between the anode 3 and the cathode 4. The light emitting unit 10 includes a light emitting layer 5. A hole transporting zone 6 (a hole injecting layer, a hole transporting layer, etc.) is provided between the light emitting layer 5 and the anode 3, and an electron transporting zone 7 (an electron injecting layer, an electron transporting layer, etc.) is provided between the light emitting layer 5 and the cathode 4. In addition, an electron blocking layer (not shown) may be provided on the anode 3 side of the light emitting layer 5, and a hole blocking layer (not shown) may be provided on the cathode 4 side of the light emitting layer 5. Thus, electrons and holes can be confined in the light emitting layer 5 to further increase the production efficiency of excitons in the light emitting layer 5.

FIG. 2 is a schematic illustration showing another configuration of the organic EL device according to an aspect of the present invention. An organic EL device 11 includes the substrate 2, the anode 3, the cathode 4, and a light emitting unit 20 disposed between the anode 3 and the cathode 4. The light emitting unit 20 includes the light emitting layer 5. A hole transporting zone disposed between the anode 3 and the light emitting layer 5 is formed of a hole injecting layer 6a, a first hole transporting layer 6b, and a second hole transporting layer 6c. An electron transporting zone disposed between the light emitting layer 5 and the cathode 4 is formed of a first electron transporting layer 7a and a second electron transporting layer 7b.

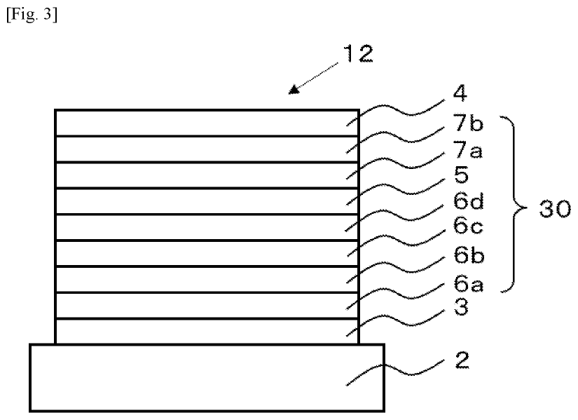

FIG. 3 is a schematic illustration showing still another configuration of the organic EL device according to an aspect of the present invention. An organic EL device 12 includes the substrate 2, the anode 3, the cathode 4, and a light emitting unit 30 disposed between the anode 3 and the cathode 4. The light emitting unit 30 includes the light emitting layer 5. A hole transporting zone disposed between the anode 3 and the light emitting layer 5 is formed of the hole injecting layer 6a, the first hole transporting layer 6b, the second hole transporting layer 6c, and a third hole transporting layer 6d. An electron transporting zone disposed between the light emitting layer 5 and the cathode 4 is formed of the first electron transporting layer 7a and the second electron transporting layer 7b.

In the present invention, a host combined with a fluorescent dopant material (a fluorescent light emitting material) is referred to as a fluorescent host, and a host combined with a phosphorescent dopant material is referred to as a phosphorescent host. The fluorescent host and the phosphorescent host are not distinguished from each other merely by the molecular structures thereof. Specifically, the phosphorescent host means a material that forms a phosphorescent light emitting layer containing a phosphorescent dopant, but does not mean unavailability as a material that forms a fluorescent light emitting layer. The same also applies to the fluorescent host.

### Substrate

The substrate is used as a support of the organic EL device. As the substrate, for example, a plate of glass, quartz, and plastic, can be used. A flexible substrate may also be used. Examples of the flexible substrate include a plastic substrate made of polyimide, polycarbonate, polyarylate, polyether sulfone, polypropylene, polyester, polyvinyl fluoride, or polyvinyl chloride. An inorganic vapor deposition film can also be used.

Anode A metal, an alloy, an electrically conductive compound, a mixture thereof, and the like which have a high work function (specifically 4.0 eV or more) are preferably used for the anode formed on the substrate. Specific examples thereof include indium oxide-tin oxide (ITO: indium tin oxide), indium oxide-tin oxide containing silicon or silicon oxide, indium oxide-zinc oxide, indium oxide containing tungsten oxide and zinc oxide, and graphene. Besides, examples thereof include gold (Au), platinum (Pt), nickel (Ni), tungsten (W), chromium (Cr), molybdenum (Mo), iron (Fe), cobalt (Co), copper (Cu), palladium (Pd), titanium (Ti), or nitrides of these metals (for example, titanium nitride).

These materials are usually formed into a film by a sputtering method. For example, indium oxide-zinc oxide can be formed by using a target of indium oxide in which 1 to 10% by weight of zinc oxide with respected to the indium oxide is added, and indium oxide containing tungsten oxide and zinc oxide can be formed by using a target of indium oxide in which 0.5 to 5% by weight of tungsten oxide and 0.1 to 1% by weight of zinc oxide with respect to the indium oxide are incorporated, through a sputtering method. Besides, the manufacturing may be performed by a vacuum vapor deposition method, a coating method, an inkjet method, a spin coating method, or the like.

### Hole Transporting Zone

As described above, the organic layers may include the hole transporting zone between the anode and the light emitting layer. The hole transporting zone is composed of the hole injecting layer, the hole transporting layer, the electron blocking layer, etc. The hole transporting zone preferably contains the inventive compound (1). The inventive compound (1) is preferably contained in at least one layer of these layers which constitute the hole transporting layer, and in particular, the inventive compound (1) is more preferably contained in the hole transporting layer.

The hole injecting layer formed in contact with the anode is formed by using a material that facilitates hole injection regardless of the work function of the anode, and thus, it is possible to use materials that are generally used as an electrode material (for example, metals, alloys, electrically conductive compounds, or mixtures thereof, or elements belonging to Group 1 or 2 of the periodic table of the elements).

It is also possible to use elements belonging to Group 1 or 2 of the periodic table of the elements, which are materials having low work functions, specifically, alkali metals, such as lithium (Li) and cesium (Cs), alkaline earth metals, such as magnesium (Mg), calcium (Ca), and strontium (Sr), and alloys containing these metals (such as MgAg and AlLi), rare earth metals, such as europium (Eu) and ytterbium (Yb), and alloys containing these metals, and the like. When the anode is formed by using the alkali metals, the alkaline earth metals, and alloys containing these metals, a vacuum vapor deposition method or a sputtering method can be used. Furthermore, when a silver paste or the like is used, a coating method, an inkjet method, or the like can be used.

### Hole Injecting Layer

The hole injecting layer is a layer containing a material having a high hole injection capability (a hole injecting material) and is formed between the anode and the light emitting layer, or between the hole transporting layer, if exists, and the anode.

As the hole injecting material other than the inventive compound, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, and the like can be used.

Examples of the hole injecting layer material also include aromatic amine compounds which are low-molecular weight organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

High-molecular weight compounds (such as oligomers, dendrimers, and polymers) can also be used. Examples thereof include high-molecular weight compounds, such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). In addition, high-molecular weight compounds with an acid added thereto, such as poly(3,4-ethylenedioxythiophene)/poly(styrenesulfonic acid) (PEDOT/PSS), and polyaniline/poly(styrenesulfonic acid) (PAni/PSS), can also be used.

Furthermore, it is also preferred to use an acceptor material, such as a hexaazatriphenylene (HAT) compound represented by the following formula (K).

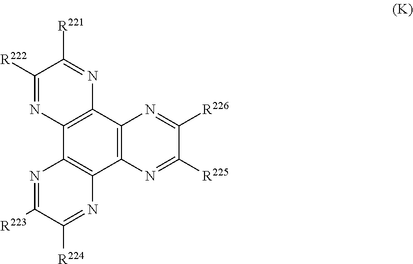

(In the formula, R²²¹ to R²²⁶ each independently represent a cyano group, —CONH₂, a carboxy group, or —COOR²²⁷ (R²²⁷ represents an alkyl group having 1 to 20 carbon atoms or a cycloalkyl group having 3 to 20 carbon atoms). In addition, adjacent two selected from R²²¹ and R²²², R²²³ and R²²⁴, and R²²⁵ and R²²⁶ may be bonded to each other to form a group represented by —CO—O—CO—.)

Examples of R²²⁷ include a methyl group, an ethyl group, an n-propyl group, an isopropyl group, an n-butyl group, an isobutyl group, a t-butyl group, a cyclopentyl group, and a cyclohexyl group.

### Hole Transporting Layer

The hole transporting layer is a layer containing a material having a high hole transporting capability (a hole transporting material) and is formed between the anode and the light emitting layer, or between the hole injecting layer, if exists, and the light emitting layer. The inventive compound may be used alone or in combination with the compounds described below, in the hole transporting layer.

The hole transporting layer may have a single-layer structure or a multilayer structure including two or more layers. For example, the hole transporting layer may have a two-layer structure including a first hole transporting layer (anode side) and a second hole transporting layer (cathode side). In other words, the hole transporting zone may include a first hole transporting layer on the anode side and a second hole transporting layer on the cathode side. The hole transporting layer may have a three-layer structure including a first hole transporting layer (anode side), a second hole transporting layer, and a third hole transporting layer (cathode side). In other words, the hole transporting zone may include a first hole transporting layer on the anode side, a third hole transporting layer on the cathode side, and a second hole transporting layer disposed between the two layers. In an aspect of the present invention, the hole transporting layer of the single-layer structure is preferably disposed adjacent to the light emitting layer, and the hole transporting layer that is closest to the cathode in the multilayer structure, such as the second hole transporting layer in the hole transporting layer of the two-layer structure or the third hole transporting layer in the hole transporting layer of the three-layer structure, is preferably disposed adjacent to the light emitting layer. In another aspect of the present invention, an electron blocking layer as described later or the like may be interposed between the hole transporting layer of the single-layer structure and the light emitting layer or between the hole transporting layer that is closest to the light emitting layer in the multilayer structure and the light emitting layer. As described above, when the hole transporting layer has a multilayer structure including two or more hole transporting layers, the hole transporting layer that is adjacent to the light emitting layer in the multilayer structure can be used as an electron blocking layer.

In an aspect of the organic electroluminescent device according to the present invention, at least one of the first hole transporting layer and the second hole transporting layer in the hole transporting layer of the two-layer structure contains the inventive compound. Specifically, in the hole transporting layer of the two-layer structure, the inventive compound may be contained in one of the first hole transporting layer and the second hole transporting layer or may be contained in the both.

In an aspect of the present invention, the inventive compound is preferably contained only in the first hole transporting layer, in another aspect, the inventive compound is preferably contained only in the second hole transporting layer, and in still another aspect, the inventive compound is preferably contained in the first hole transporting layer and in the second hole transporting layer.

In another aspect of the organic electroluminescent device according to the present invention, at least one of the first hole transporting layer, the second hole transporting layer, and the third hole transporting layer in the hole transporting layer of the three-layer structure contains the inventive compound. Specifically, in the hole transporting layer of the three-layer structure, the inventive compound may be contained in any one of the first hole transporting layer, the second hole transporting layer, and the third hole transporting layer, may be contained in any two thereof, or may be contained in all thereof.

More specifically, any of an aspect in which the inventive compound is contained only in the first hole transporting layer, an aspect in which the inventive compound is contained only in the second hole transporting layer, an aspect in which the inventive compound is contained only in the third hole transporting layer, an aspect in which the inventive compound is contained only in the first and second hole transporting layers, an aspect in which the inventive compound is contained only in the second and third hole transporting layers, an aspect in which the inventive compound is contained only in the first and third hole transporting layers, and an aspect in which the inventive compound is contained in all the first to third hole transporting layers may be employed.

In an aspect of the present invention, the inventive compound which is contained in one or both of the first hole transporting layer and the second hole transporting layer, or the inventive compound which is contained in any one of the first to third hole transporting layers, in any two of the first to third hole transporting layers, or in all the first to third hole transporting layers is preferably a protium form from the viewpoint of production cost.

The protium form refers to the inventive compound in which all the hydrogen atoms are each a protium atom.

Accordingly, the present invention encompasses an organic EL device in which one or both of the first hole transporting layer and the second hole transporting layer contain the inventive compound essentially constituted only of the protium form and an organic EL device in which any one of the first to third hole transporting layers, any two of the first to third hole transporting layers, or all of the first to third hole transporting layers contain the inventive compound essentially constituted only of the protium form. The phrase “the inventive compound essentially constituted only of the protium form” means that the content of the protium form based on the total amount of the inventive compound is 90% by mole or more, preferably 95% by mole or more, and more preferably 99% by mole or more (each including 100%).

As a material for a hole transporting layer other than the inventive compound, for example, an aromatic amine compound, a carbazole derivative, or an anthracene derivative, can be used.

Examples of the aromatic amine compound include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB). The compound has a hole mobility of 10⁻⁶ cm²/Vs or more.

Examples of the carbazole derivative include 4,4′-di(9-carbazolyl)biphenyl (abbreviation: CBP), 9-[4-(9-carbazolyl)phenyl]-10-phenyl anthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenyl anthracene (abbreviation: DPAnth).

High molecular weight compounds, such as poly(N-vinylcarbazole) (abbreviation: PVK) and poly(4-vinyltriphenylamine) (abbreviation: PVTPA), can also be used.

However, compounds other than those as mentioned above may also be used so long as they are compounds high in the hole transporting capability rather than in the electron transporting capability.

### Dopant Material of Light Emitting Layer

The light emitting layer is a layer containing a material having a high light emitting property (a dopant material), and various materials can be used. For example, a fluorescent light emitting material or a phosphorescent light emitting material can be used as the dopant material. The fluorescent light emitting material is a compound that emits light from a singlet excited state, and the phosphorescent light emitting material is a compound that emits light from a triplet excited state.

Examples of a blue-based fluorescent light emitting material that can be used for the light emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), and 4-(10-phenyl-9-anthryl)-4′-(9-phenyl-9H-carbazol-3-yl)triphenylamine (abbreviation: PCBAPA).

An example of a green-based fluorescent light emitting material that can be used for the light emitting layer is an aromatic amine derivative. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazol-9-yl)phenyl]-N-phenylanthracene-2-amine (abbreviation: 2YGABPhA), and N,N,9-triphenylanthracene-9-amine (abbreviation: DPhAPhA).

Examples of a red-based fluorescent light emitting material that can be used for the light emitting layer include a tetracene derivative and a diamine derivative. Specific examples thereof include N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD).

In an aspect of the present invention, the light emitting layer preferably contains a fluorescent light emitting material (fluorescent dopant material).

Examples of a blue-based phosphorescent light emitting material that can be used for the light emitting layer include metal complexes, such as an iridium complex, an osmium complex, and a platinum complex. Specific examples thereof include bis[2-(4′,6′-difluorophenyl)pyridinato-N,C2′]iridium(III) tetrakis(1-pyrazolyl)borate (abbreviation: FIr6), bis[2-(4′,6′-difluorophenyl)pyridinato-N,C2′]iridium(III) picolinate (abbreviation: FIrpic), bis[2-(3′,5′-bistrifluoromethylphenyl)pyridinato-N,C2′]iridium(III) picolinate (abbreviation: Ir(CF3ppy)2(pic)), and bis[2-(4′,6′-difluorophenyl)pyridinato-N,C2′]iridium(III) acetylacetonate (abbreviation: FIracac).

An example of a green-based phosphorescent light emitting material that can be used for the light emitting layer is an iridium complex. Examples thereof include tris(2-phenylpyridinato-N,C2′)iridium(III) (abbreviation: Ir(ppy)3), bis(2-phenylpyridinato-N,C2′)iridium(III) acetylacetonate (abbreviation: Ir(ppy)2(acac)), bis(1,2-diphenyl-1H-benzimidazolato)iridium(III) acetylacetonate (abbreviation: Ir(pbi)2(acac)), and bis(benzo[h]quinolinato)iridium(III) acetylacetonate (abbreviation: Ir(bzq)2(acac)).

Examples of a red-based phosphorescent light emitting material that can be used for the light emitting layer include metal complexes, such as an iridium complex, a platinum complex, a terbium complex, and a europium complex. Specific examples thereof include organic metal complexes, such as bis[2-(2′-benzo[4,5-a]thienyl)pyridinato-N,C3′]iridium(III) acetylacetonate (abbreviation: Ir(btp)2(acac)), bis(1-phenylisoquinolinato-N,C2′)iridium(III) acetylacetonate (abbreviation: Ir(piq)2(acac)), (acetylacetonate)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrin platinum(II) (abbreviation: PtOEP).

In addition, rare earth metal complexes, such as tris(acetylacetonate)(monophenanthroline)terbium(III) (abbreviation: Tb(acac)3(Phen)), tris(1,3-diphenyl-1,3-propanedionato)(monophenanthroline)europium(III) (abbreviation: Eu(DBM)3(Phen)), and tris[1-(2-thenoyl)-3,3,3-trifluoroacetonato](monophenanthroline)europium(III) (abbreviation: Eu(TTA)3(Phen)), which emit light from rare earth metal ions (electron transition between different multiplicities), can be used as a phosphorescent light emitting material.

In an aspect of the present invention, the light emitting layer preferably contains a phosphorescent light emitting material (phosphorescent dopant material).

### Host Material of Light Emitting Layer

The light emitting layer may have a configuration in which the aforementioned dopant material is dispersed in another material (a host material). A material that has a higher lowest unoccupied orbital level (LUMO level) and a lower highest occupied orbital level (HOMO level) than the dopant material is preferably used.

As a host material, for example,
(1) a metal complex, such as an aluminum complex, a beryllium complex, or a zinc complex,
(2) a heterocyclic compound, such as an oxadiazole derivative, a benzimidazole derivative, or a phenanthroline derivative,
(3) a condensed aromatic compound, such as a carbazole derivative, an anthracene derivative, a phenanthrene derivative, a pyrene derivative, or a chrysene derivative, or
(4) an aromatic amine compound, such as a triarylamine derivative or a condensed polycyclic aromatic amine derivative is used.

For example,
metal complexes, such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), and bathocuproin (abbreviation: BCP);
condensed aromatic compounds, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and
aromatic amine compounds, such as N,N-diphenyl-9-[4-(10-phenyl-9-anthryl)phenyl]-9H-carbazole-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N,9-diphenyl-N-{4-[4-(10-phenyl-9-anthryl)phenyl]phenyl}-9H-carbazole-3-amine (abbreviation: PCAPBA), N-(9,10-diphenyl-2-anthryl)-N,9-diphenyl-9H-carbazole-3-amine (abbreviation: 2PCAPA), 4,4′-bis[N-(1-naphthyl)-N-phenylamino]biphenyl (abbreviation: NPB or u-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB) can be used. A plurality of host materials may be used.

In particular, in the case of a blue fluorescent device, it is preferred to use the following anthracene compounds as the host material.

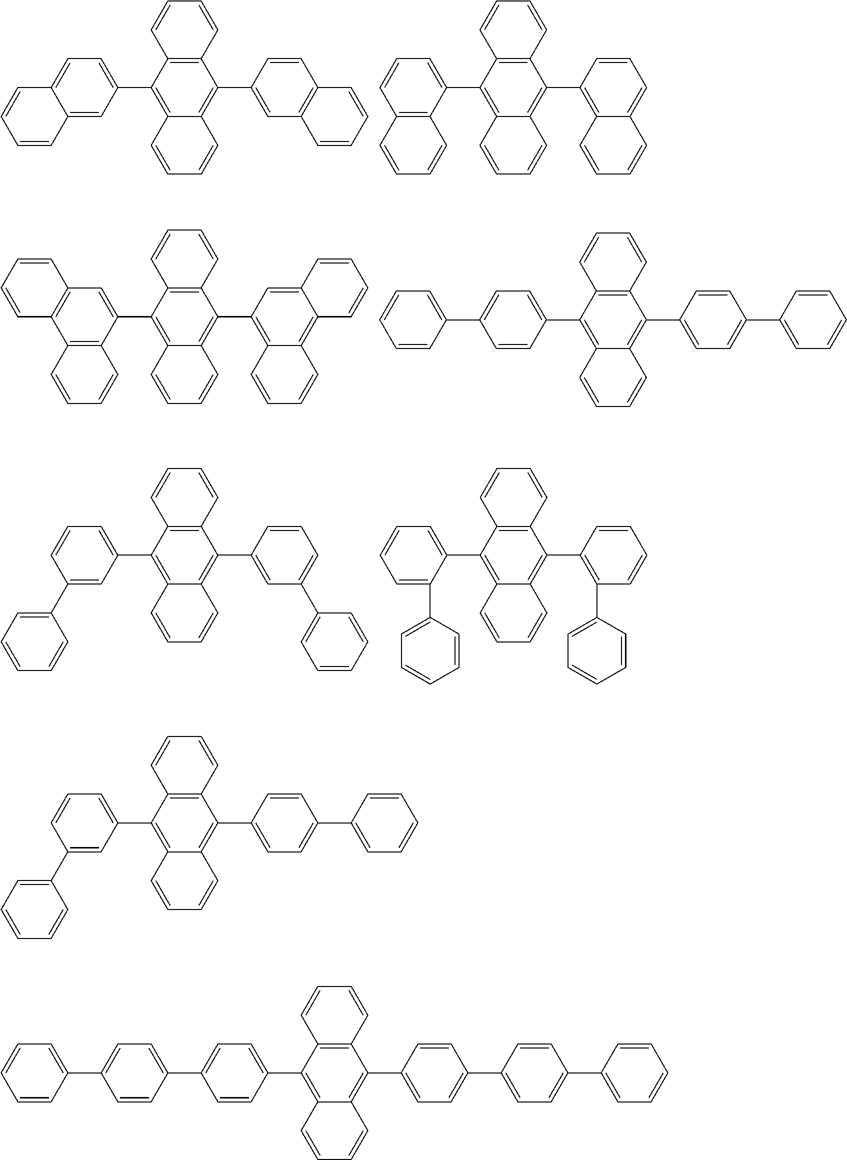

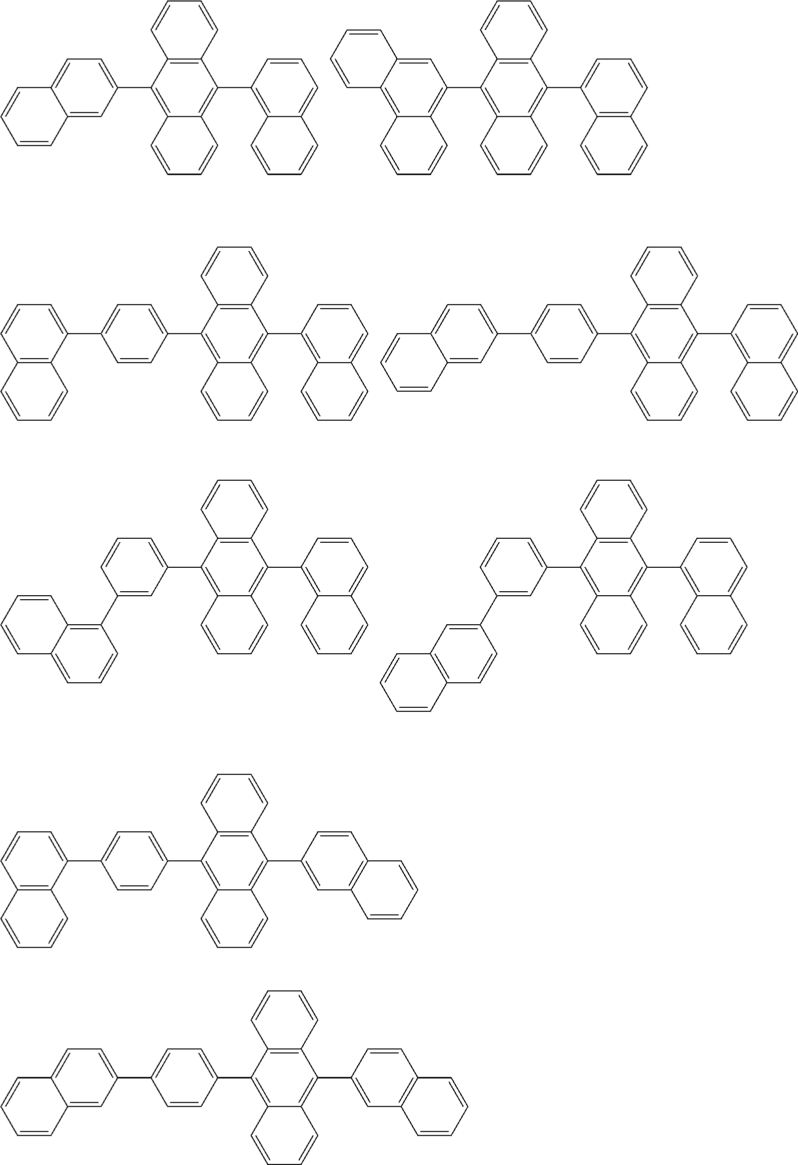

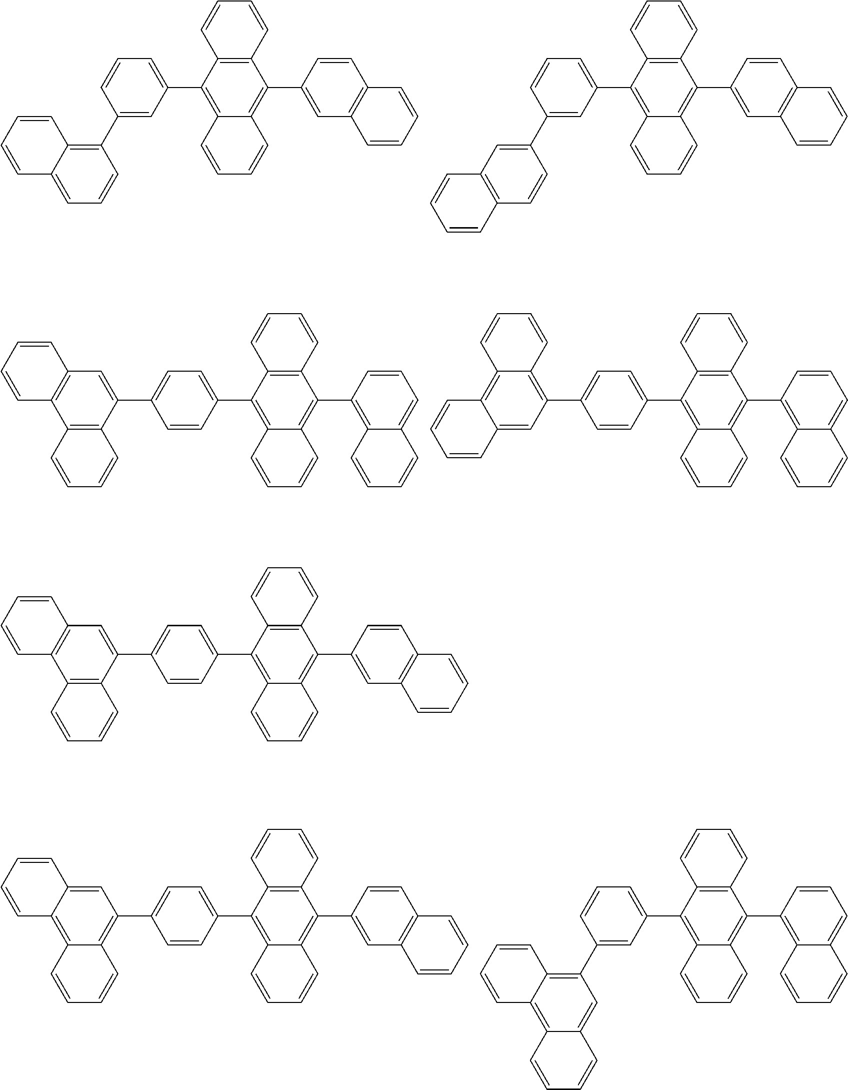

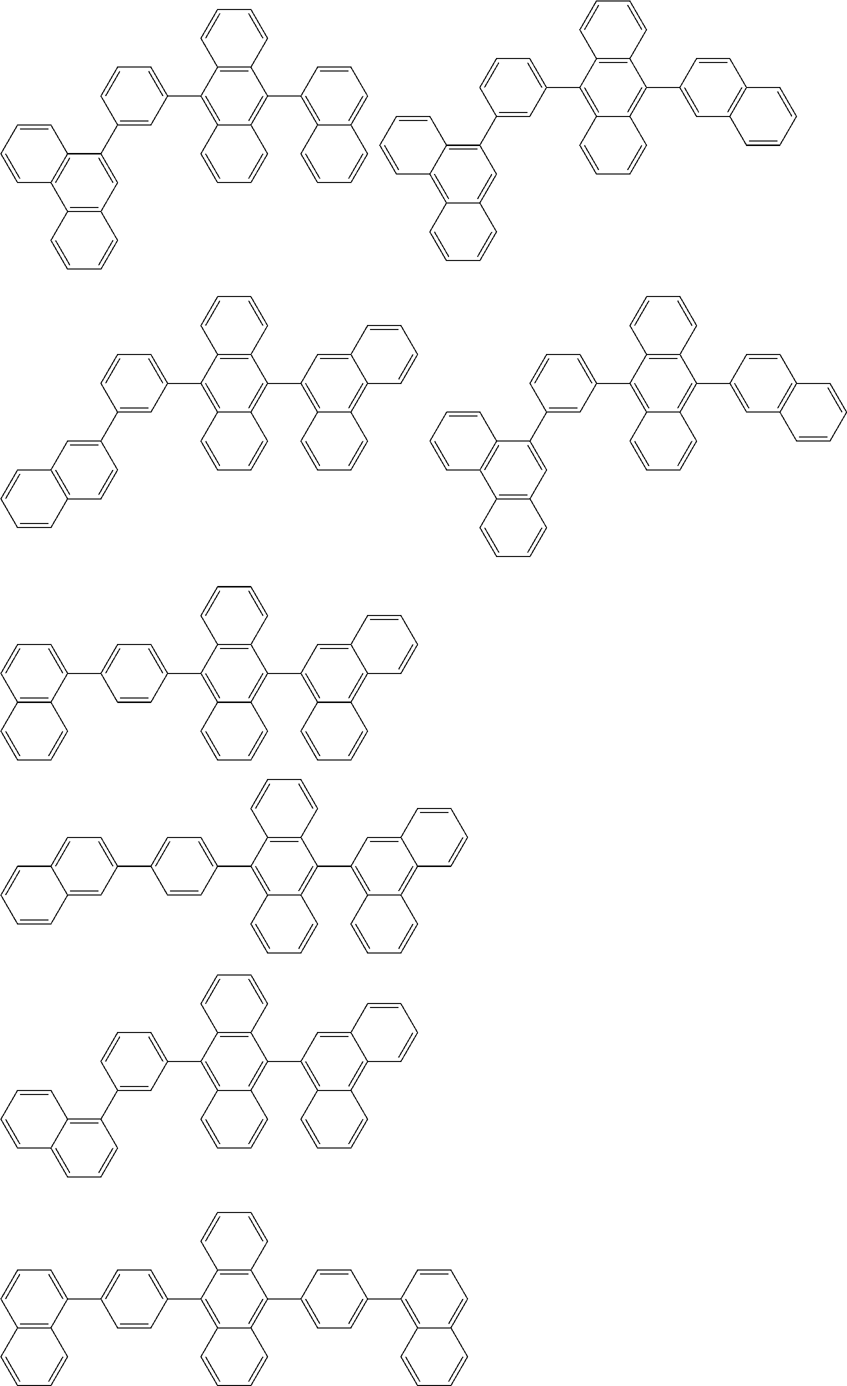

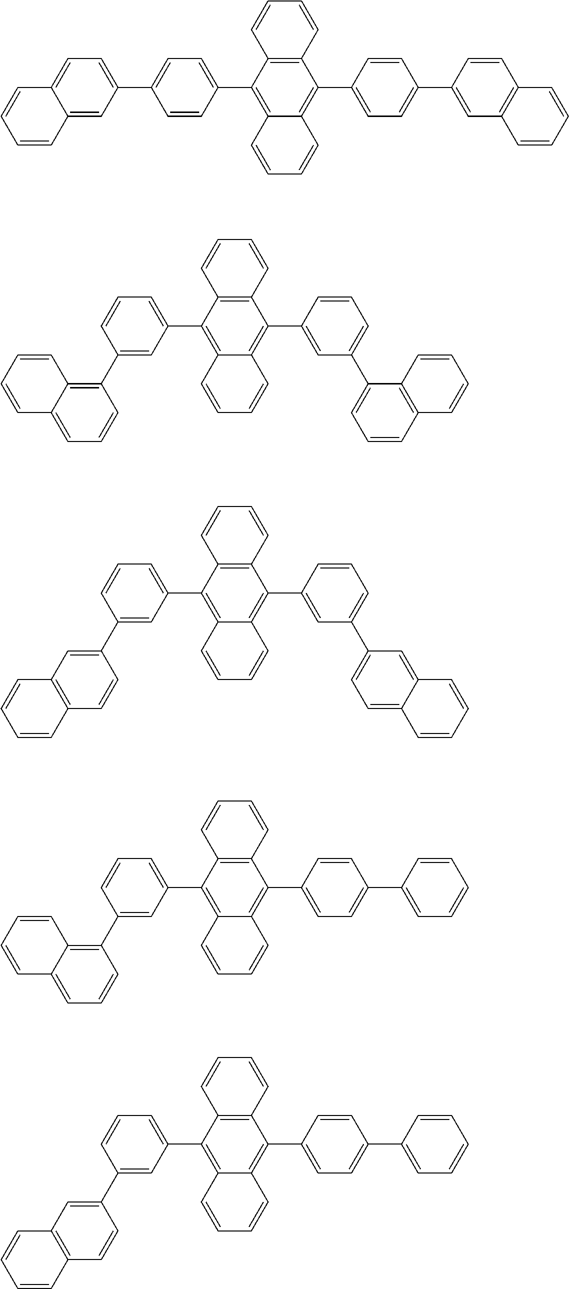

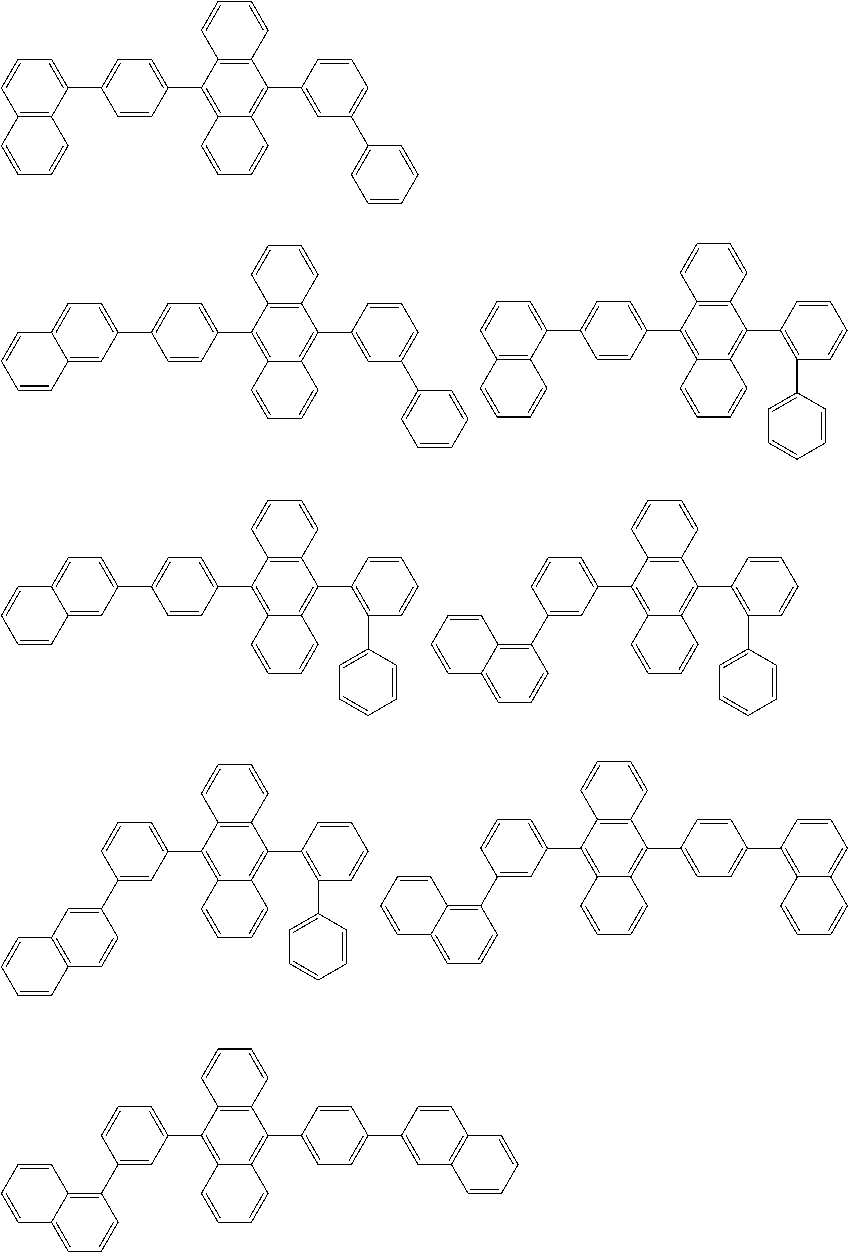

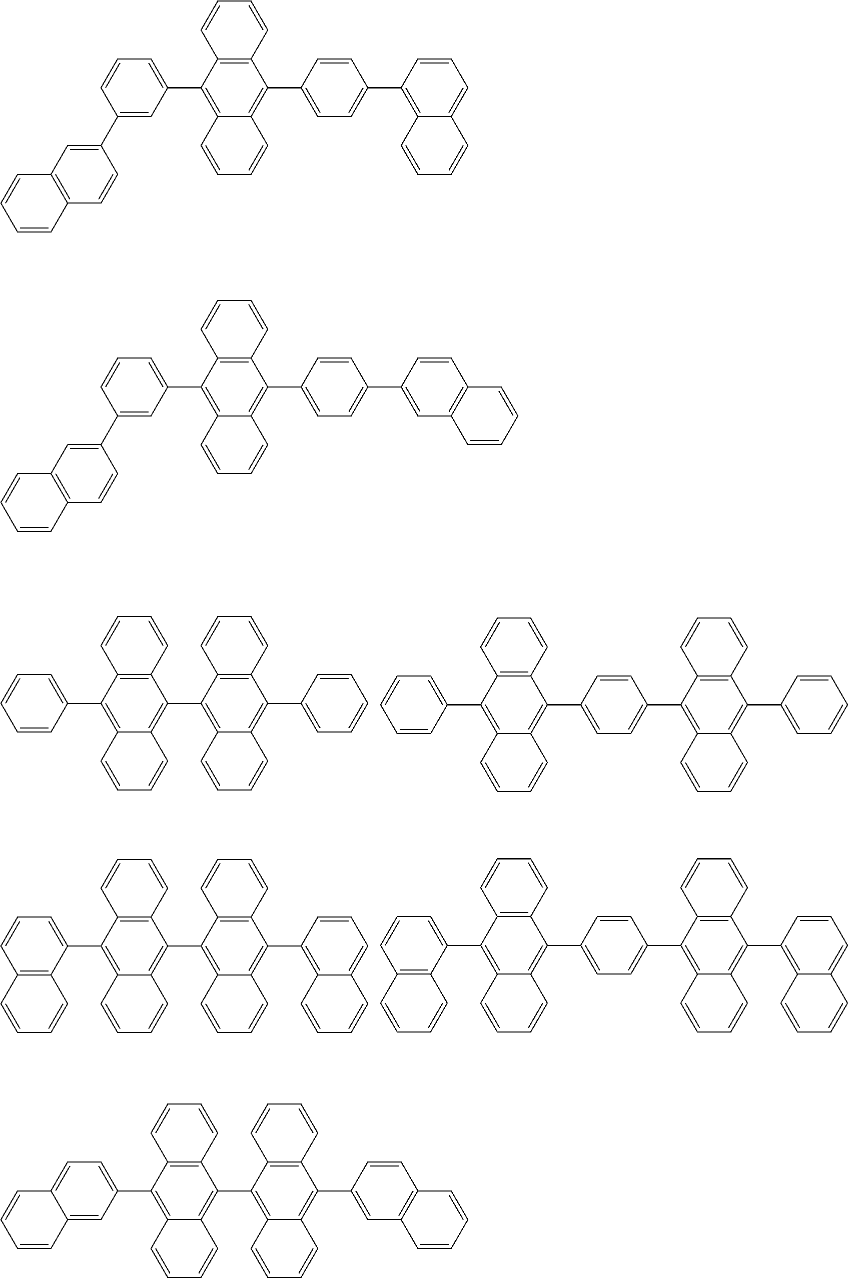

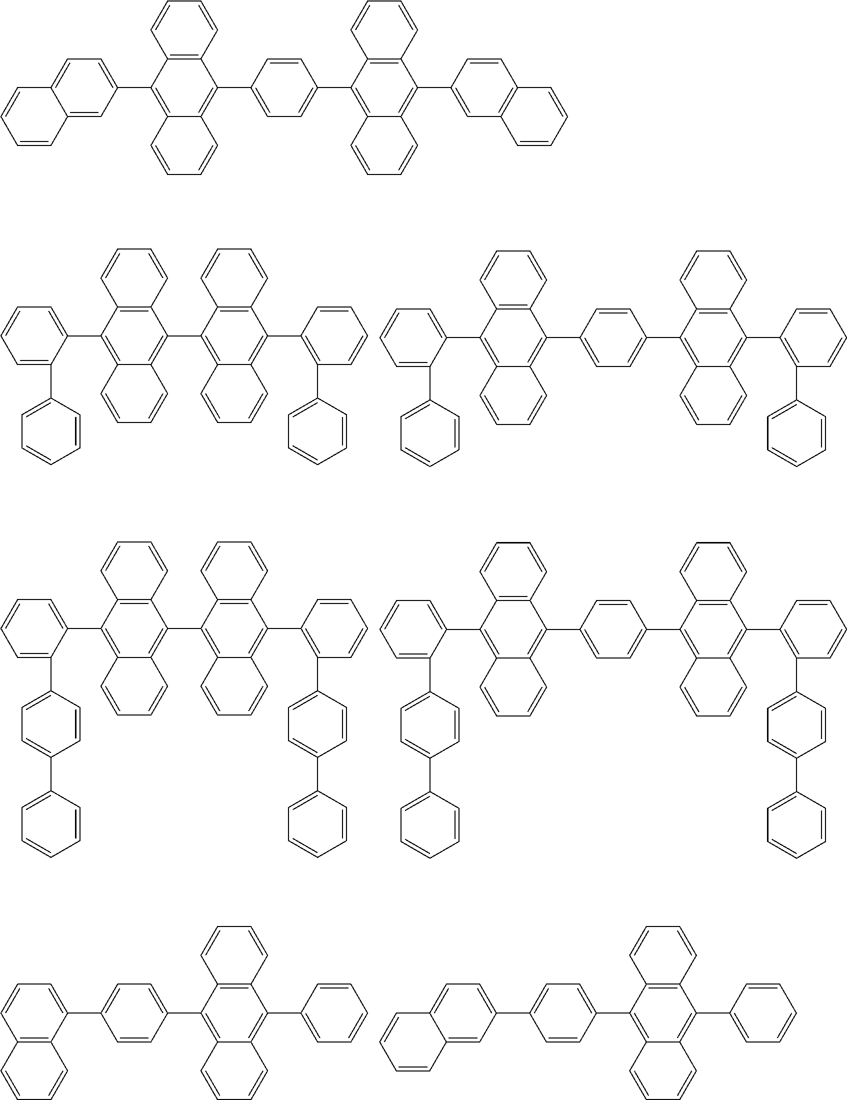

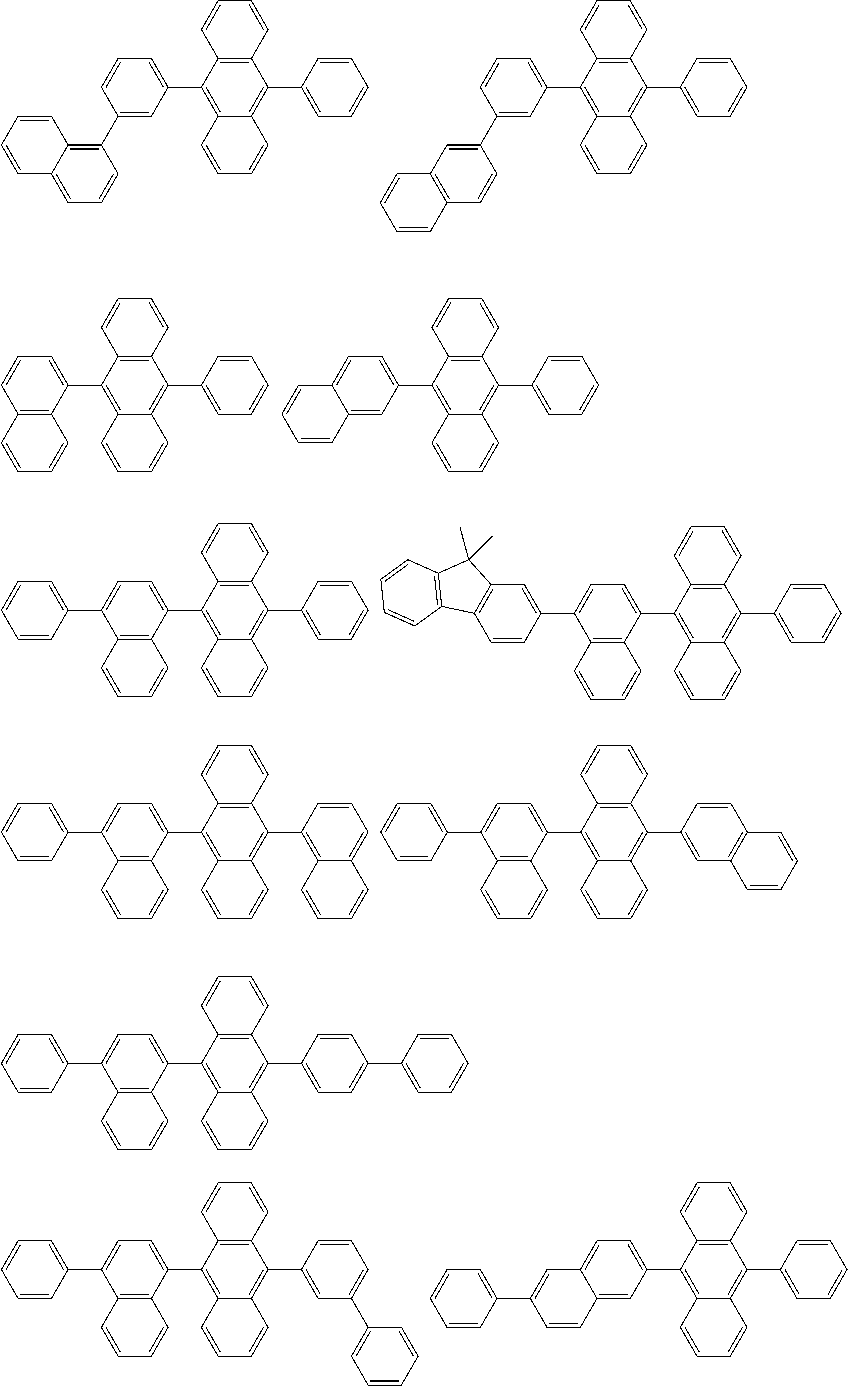

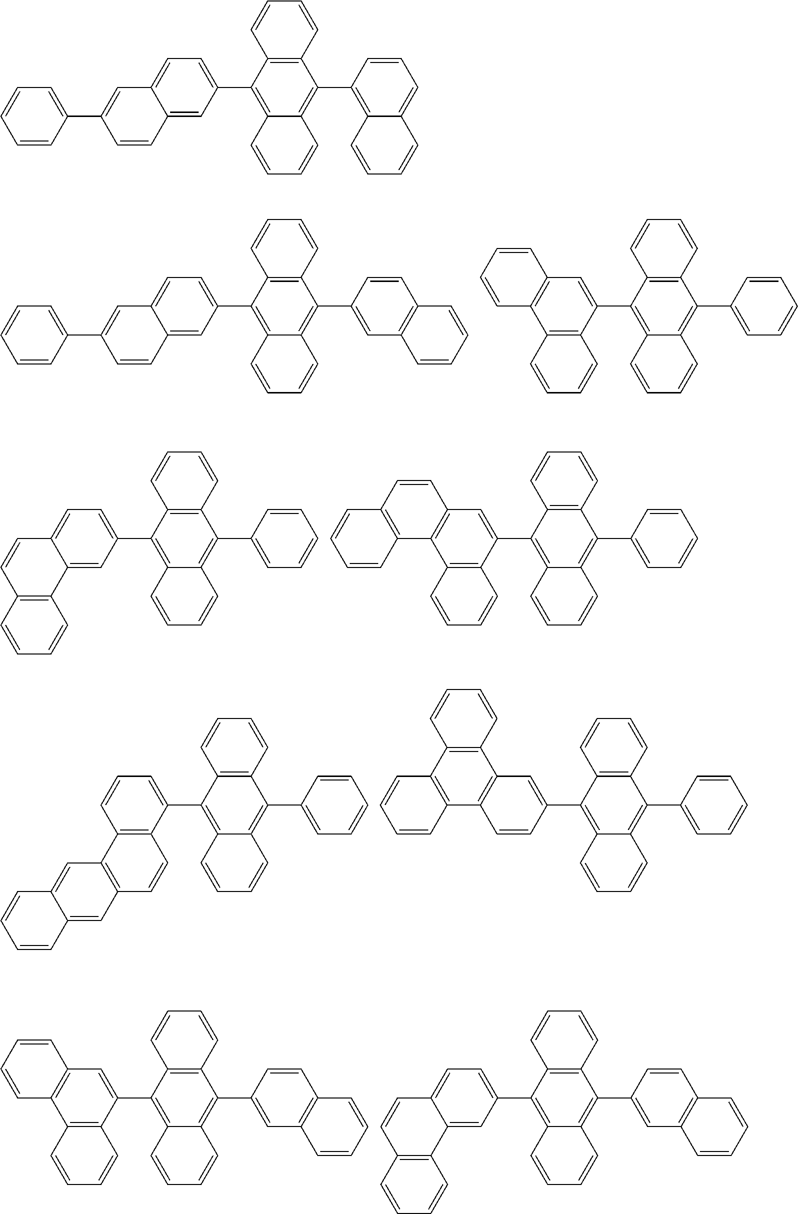

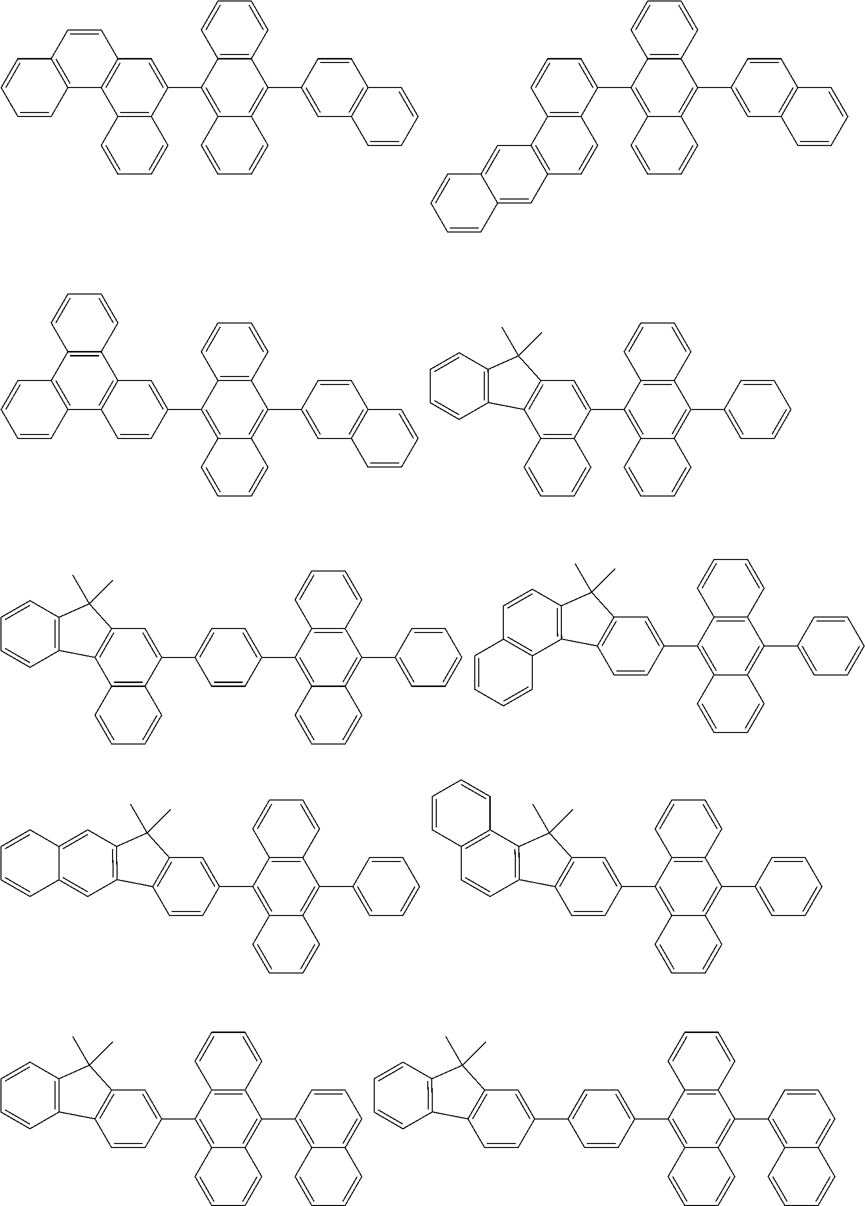

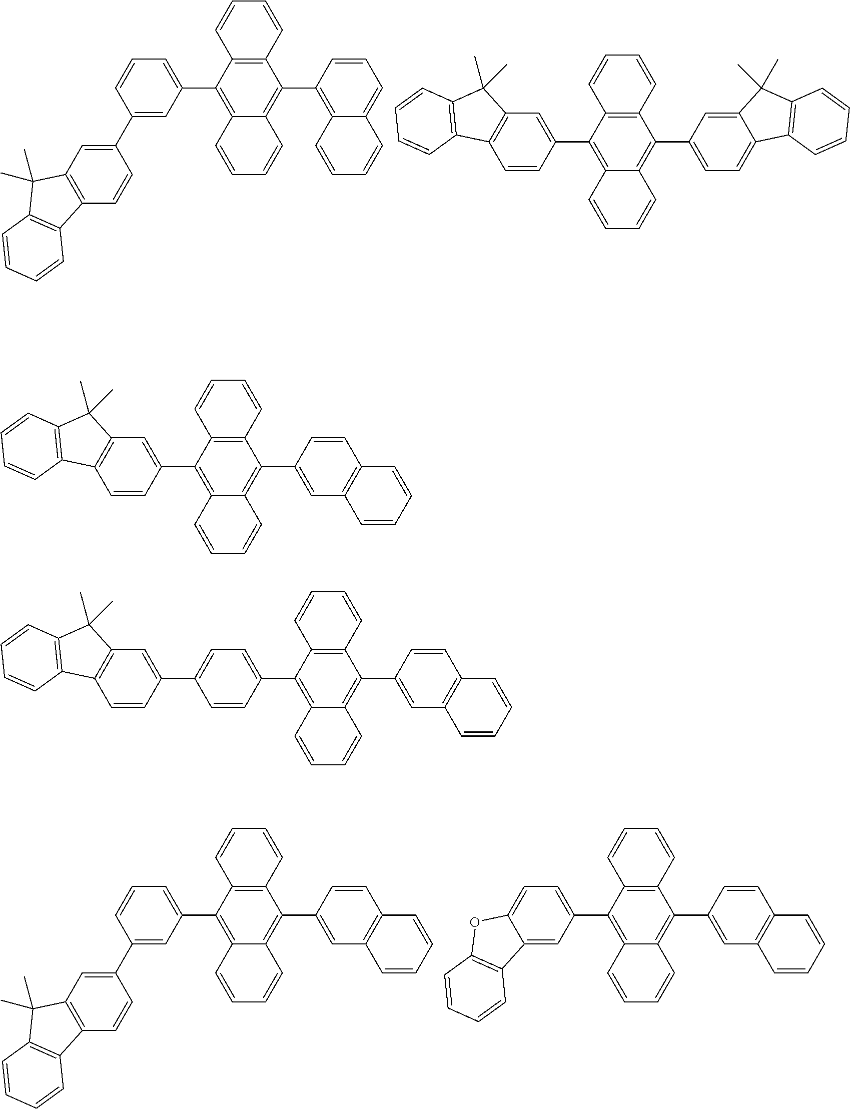

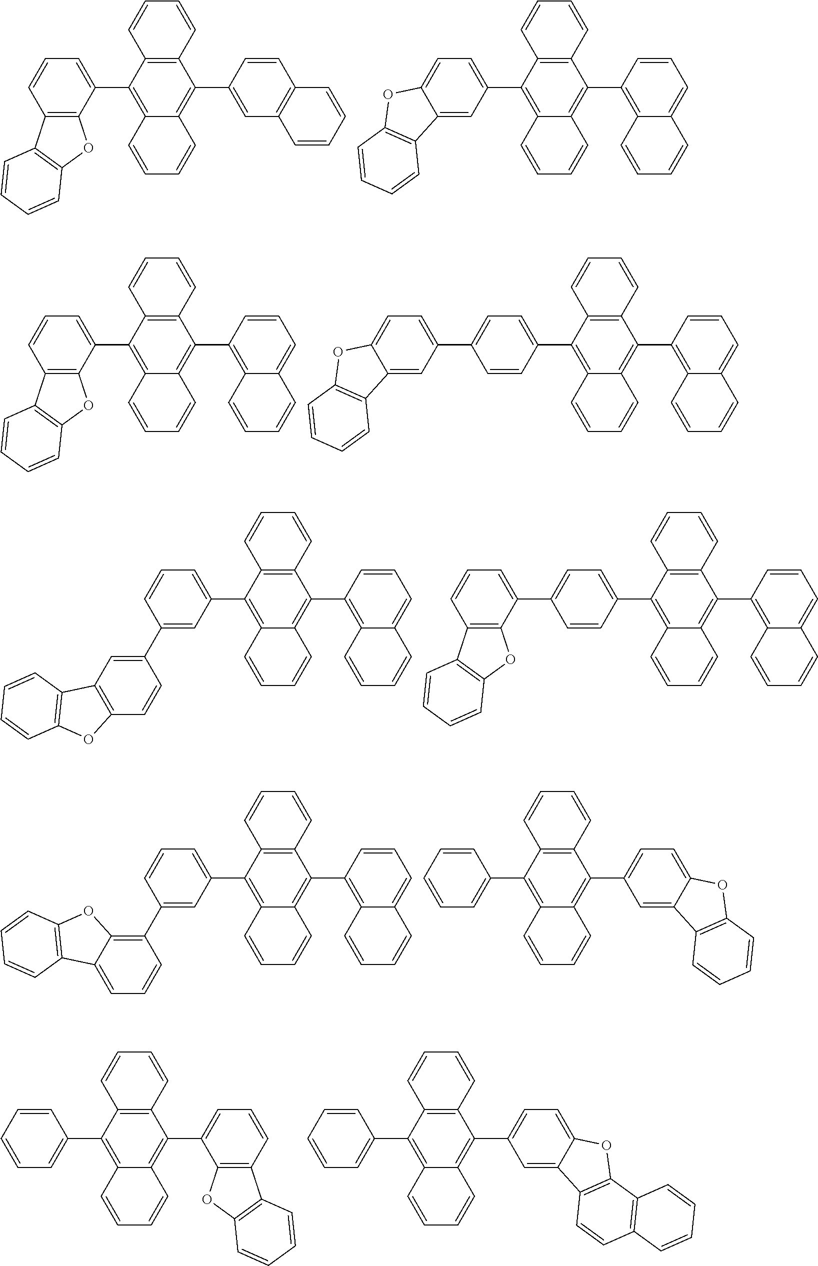

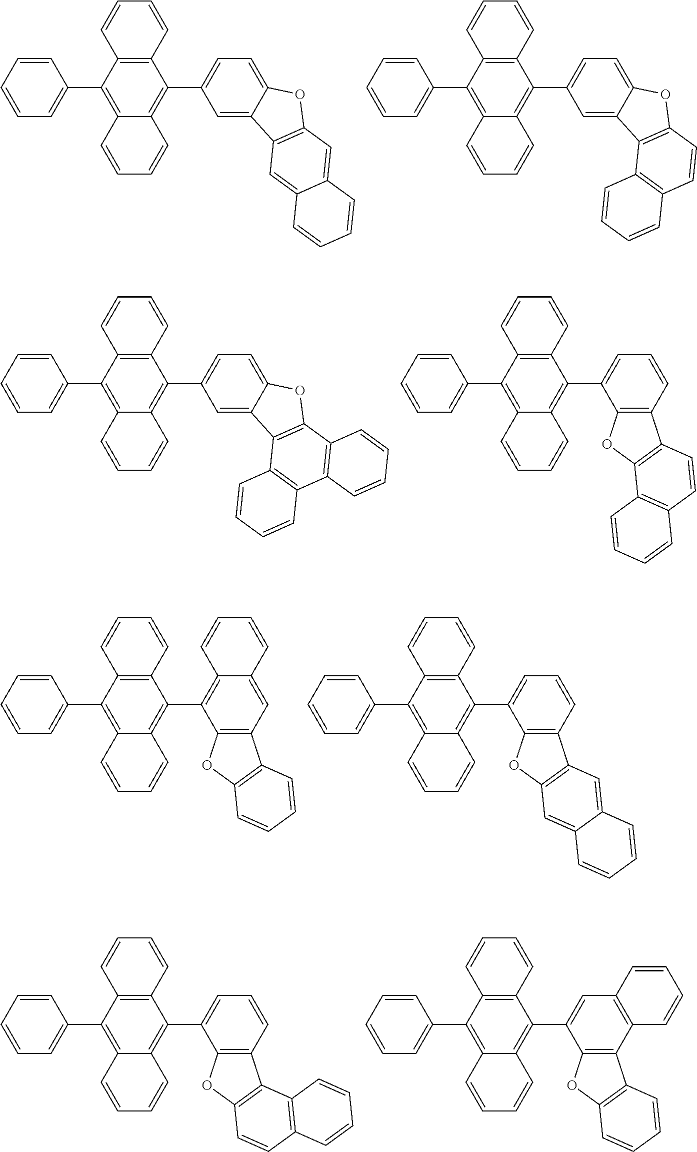

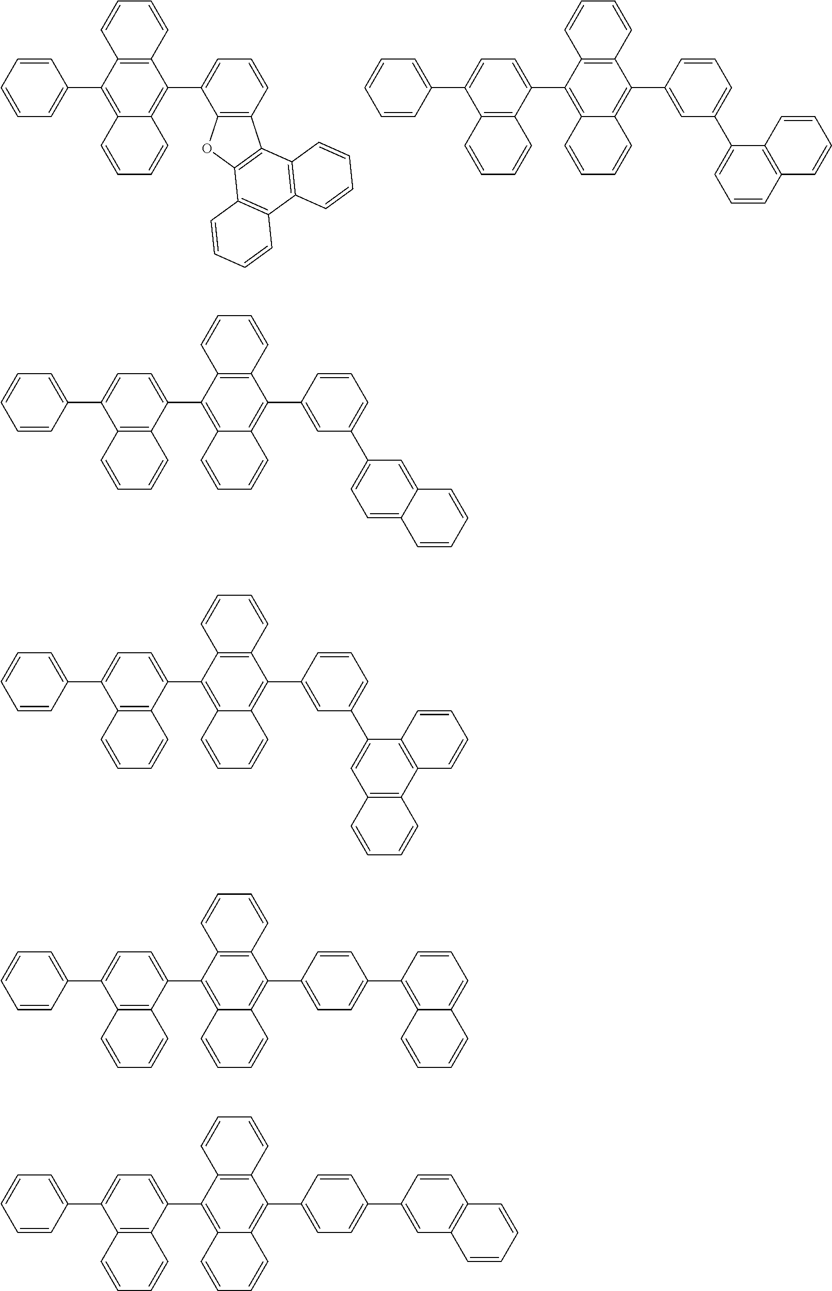

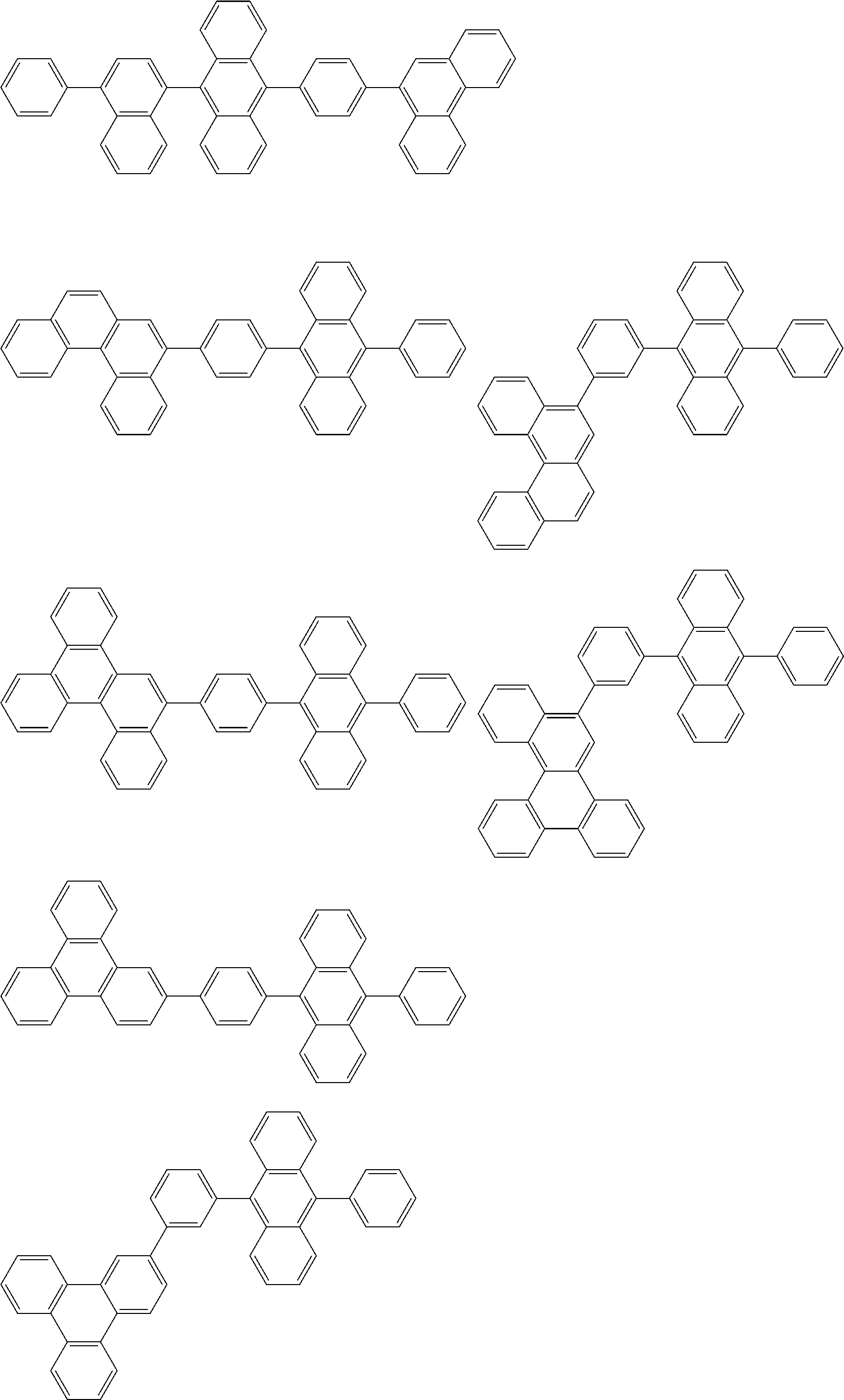

### Electron Transporting Layer

The electron transporting layer is a layer containing a material having a high electron transporting capability (an electron transporting material) and is formed between the light emitting layer and the cathode, or between the electron injecting layer, if exists, and the light emitting layer.

The electron transporting layer may have a single layer structure or a multilayer structure including two or more layers. For example, the electron transporting layer may have a two-layer structure including a first electron transporting layer (anode side) and a second electron transporting layer (cathode side). In an aspect of the present invention, the electron transporting layer of the single layer structure is preferably disposed adjacent to the light emitting layer, and the electron transporting layer that is closest to the anode in the multilayer structure, such as the first electron transporting layer of the two-layer structure, is preferably disposed adjacent to the light emitting layer. In another aspect of the present invention, a hole blocking layer as described later or the like may be interposed between the electron transporting layer of the single layer structure and the light emitting layer or between the electron transporting layer that is closest to the light emitting layer in the multilayer structure and the light emitting layer.

In the electron transporting layer, for example,
(1) a metal complex, such as an aluminum complex, a beryllium complex, or a zinc complex,
(2) a heteroaromatic compounds, such as an imidazole derivative, a benzimidazole derivative, an azine derivative, a carbazole derivative, or a phenanthroline derivative, or
(3) a high molecular weight compound can be used.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(ptert-butylphenyl)-1,3,4-oxadiazol-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproin (abbreviation: BCP), and 4,4′-bis(5-methylbenzoxazol-2-yl)stilbene (abbreviation: BzOs).

Examples of the high molecular weight compound include poly[(9,9-dihexylfluorene-2,7-diyl)-co-(pyridine-3,5-diyl)] (abbreviation: PF-Py) and poly[(9,9-dioctylfluorene-2,7-diyl)-co-(2,2′-bipyridine-6,6′-diyl)] (abbreviation: PF-BPy).

The above materials are materials having an electron mobility of 10⁻⁶ cm²/Vs or more. Materials other than those mentioned above may also be used in the electron transporting layer so long as they are materials high in the electron transporting capability rather than in the hole transporting capability.

### Electron Injecting Layer

The electron injecting layer is a layer containing a material having a high electron injection capability. For the electron injecting layer, alkali metals, such as lithium (Li) and cesium (Cs), alkaline earth metals, such as magnesium (Mg), calcium (Ca), and strontium (Sr), rare earth metals, such as europium (Eu) and ytterbium (Yb), and compounds containing these metals can be used. Examples of the compounds include an alkali metal oxide, an alkali metal halide, an alkali metal-containing organic complex, an alkaline earth metal oxide, an alkaline earth metal halide, an alkaline earth metal-containing organic complex, a rare earth metal oxide, a rare earth metal halide, and a rare earth metal-containing organic complex. A plurality of these compounds can be used in mixture.

Besides, a material having an electron transporting capability in which an alkali metal, an alkaline earth metal, or a compound thereof is incorporated, specifically, Alq in which magnesium (Mg) is incorporated and the like, may be used. In this case, electron injection from the cathode can be more efficiently performed.

Alternatively, in the electron injecting layer, a composite material obtained by mixing an organic compound with an electron donor may be used. Such a composite material is excellent in the electron injection capability and the electron transporting capability because the organic compound receives electrons from the electron donor. In this case, the organic compound is preferably a material excellent in transporting received electrons, and specifically, the aforementioned materials to constitute the electron transporting layer (such as a metal complex and a heteroaromatic compound) can be used. The material of the electron donor is simply a material having an electron donation property for the organic compound. Specifically, alkali metals, alkaline earth metals, and rare earth metals are preferred, and examples thereof include lithium, cesium, magnesium, calcium, erbium, and ytterbium. In addition, an alkali metal oxide and an alkaline earth metal oxide are preferred, and examples thereof include lithium oxide, calcium oxide, and barium oxide. A Lewis base, such as magnesium oxide, can also be used. An organic compound, such as tetrathiafulvalene (abbreviation: TTF), can also be used.

### Cathode

A metal, an alloy, an electrically conductive compound, a mixture thereof, and the like which have a low work function (specifically 3.8 eV or less) are preferably used for the cathode. Specific examples of such a cathode material include elements belonging to group 1 or 2 of the periodic table of the elements, specifically, alkali metals, such as lithium (Li) and cesium (Cs), alkaline earth metals, such as magnesium (Mg), calcium (Ca), and strontium (Sr), and alloys containing these metals (such as MgAg, and AlLi), and rare earth metals, such as europium (Eu) and ytterbium (Yb), and alloys containing these metals.

When the cathode is formed by using an alkali metal, an alkaline earth metal, and an alloy containing these metals, a vacuum vapor deposition method or a sputtering method can be adopted. When a silver paste or the like is used, a coating method, an inkjet method, or the like can be adopted.

By providing the electron injecting layer, the cathode can be formed using various electrically conductive materials, such as Al, Ag, ITO, graphene, and indium oxide-tin oxide containing silicon or silicon oxide, regardless of the magnitude of a work function. Such an electrically conductive material can be formed into a film using a sputtering method, an inkjet method, a spin coating method, or the like.

### Insulating Layer

The organic EL device applies an electric field to an ultrathin film, and thus, pixel defects are likely to occur due to leaks or short-circuiting. In order to prevent the pixel defects, an insulating layer formed of an insulating thin film layer may be inserted between a pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate of these materials may also be used.

### Space Layer

The space layer is, for example, a layer provided between a fluorescent light emitting layer and a phosphorescent light emitting layer, in the case where the fluorescent light emitting layers and the phosphorescent light emitting layers are stacked, for the purpose of preventing excitons generated in the phosphorescent light emitting layer from diffusing into the fluorescent light emitting layer or for the purpose of adjusting the carrier balance. The space layer can also be provided among the plurality of phosphorescent light emitting layers.

Since the space layer is provided between the light emitting layers, a material having both an electron transporting capability and a hole transporting capability is preferred. Also, a material having a triplet energy of 2.6 eV or more is preferred in order to prevent diffusion of the triplet energy in phosphorescent light emitting layers adjacent to each other. Examples of the material used for the space layer include the same as those used for the hole transporting layer as described above.

### Blocking Layer

The blocking layer, such as the electron blocking layer, the hole blocking layer, or the exciton blocking layer, may be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function of preventing excitons generated in the light emitting layer from diffusing into the surrounding layers to confine the excitons within the light emitting layer.

Each layer of the organic EL device may be formed by a conventionally known vapor deposition method, a coating method, or the like. For example, formation can be performed by a known method according to a vapor deposition method, such as a vacuum vapor deposition method or a molecular beam epitaxy (MBE method), or a coating method, such as a dipping method, a spin-coating method, a casting method, a bar-coating method, and a roll-coating method, using a solution of a compound for forming a layer.

The film thickness of each layer is not particularly limited, but is typically 5 nm to 10 μm, and more preferably 10 nm to 0.2 μm because in general, when the film thickness is too small, defects such as pinholes are likely to occur, and conversely, when the film thickness is too large, a high driving voltage is required and thus, the efficiency is decreased.

Examples of embodiments of the organic EL device of the present invention include a first embodiment in which the second hole transporting layer contains the compound of the present invention and the first hole transporting layer does not contain the compound of the present invention; a second embodiment in which both of the first hole transporting layer and the second hole transporting layer contain the compound of the present invention; and a third embodiment in which the first hole transporting layer contains the compound of the present invention and the second hole transporting layer does not contain the compound of the present invention.

### Electronic Appliance

The organic EL device can be used for electronic appliances, such as display components of an organic EL panel module and the like, display units of a television, a mobile phone, a personal computer, and the like, and light emitting units of lighting and a vehicular lamp.

### EXAMPLES

The present invention will be described in more detail below by reference to Examples, but the present invention is not to be limited to the following description.

### Compound Used for Production of Organic EL Device of Example 1

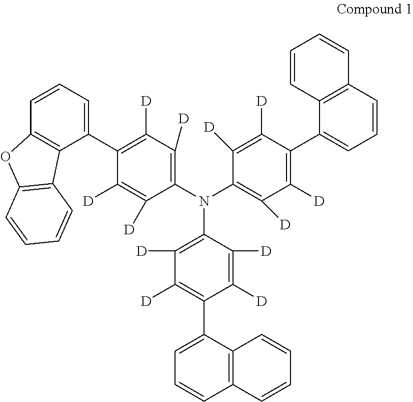 [SMILES: [2H]c1c([2H])c(N(c2c([2H])c([2H])c(-c3cccc4ccccc34)c([2H])c2[2H])c2c([2H])c([2H])c(-c3cccc4oc5ccccc5c34)c([2H])c2[2H])c([2H])c([2H])c1-c1cccc2ccccc12]

### Compound Used for Production of Organic EL Device of Example 2

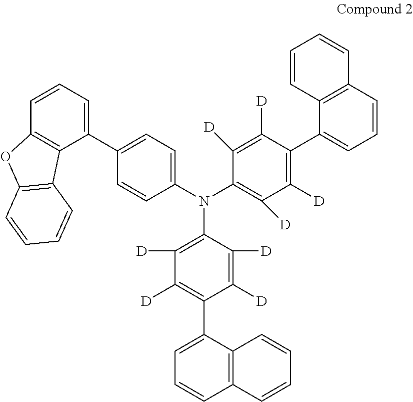 [SMILES: [2H]c1c([2H])c(N(c2ccc(-c3cccc4oc5ccccc5c34)cc2)c2c([2H])c([2H])c(-c3cccc4ccccc34)c([2H])c2[2H])c([2H])c([2H])c1-c1cccc2ccccc12]

### Compound Used for Production of Organic EL Device of Example 3

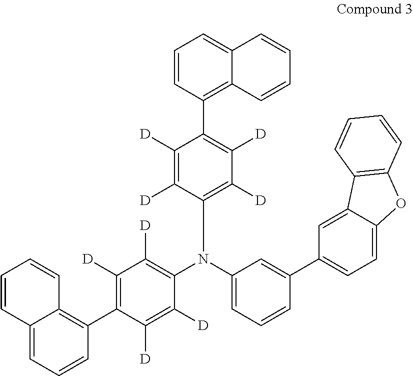 [SMILES: [2H]c1c([2H])c(N(c2cccc(-c3ccc4oc5ccccc5c4c3)c2)c2c([2H])c([2H])c(-c3cccc4ccccc34)c([2H])c2[2H])c([2H])c([2H])c1-c1cccc2ccccc12]

### Compounds Used for Production of Organic EL Device of Example 4

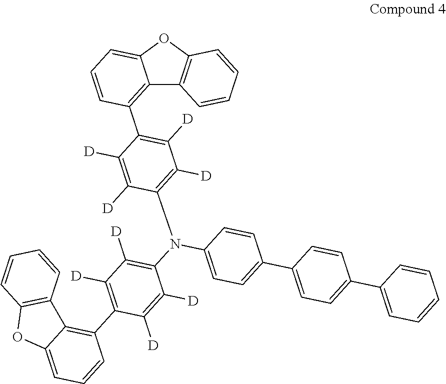 [SMILES: [2H]c1c([2H])c(N(c2ccc(-c3ccc(-c4ccccc4)cc3)cc2)c2c([2H])c([2H])c(-c3cccc4oc5ccccc5c34)c([2H])c2[2H])c([2H])c([2H])c1-c1cccc2oc3ccccc3c12]

### Comparative Compound Used for Production of Organic EL Device of Comparative Example 1

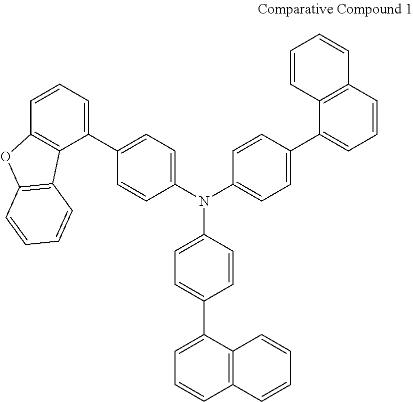 [SMILES: c1ccc2c(-c3ccc(N(c4ccc(-c5cccc6ccccc56)cc4)c4ccc(-c5cccc6oc7ccccc7c56)cc4)cc3)cccc2c1]

### Compound Used for Production of Organic EL Device of Comparative Example 2

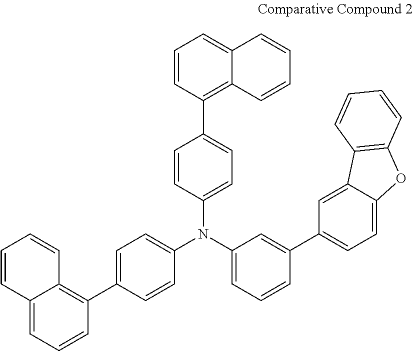 [SMILES: c1cc(-c2ccc3oc4ccccc4c3c2)cc(N(c2ccc(-c3cccc4ccccc34)cc2)c2ccc(-c3cccc4ccccc34)cc2)c1]

### Compound Used for Production of Organic EL Device of Comparative Example 3

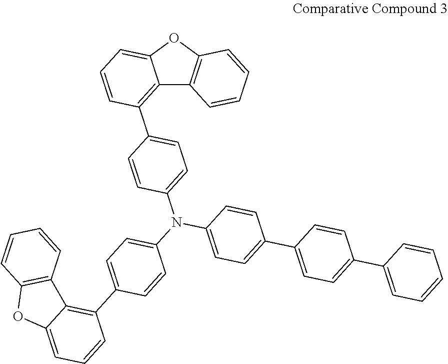 [SMILES: c1ccc(-c2ccc(-c3ccc(N(c4ccc(-c5cccc6oc7ccccc7c56)cc4)c4ccc(-c5cccc6oc7ccccc7c56)cc4)cc3)cc2)cc1]

### Other Compounds Used for Production of Organic EL Devices of Examples 1 and 2 and Comparative Example 1

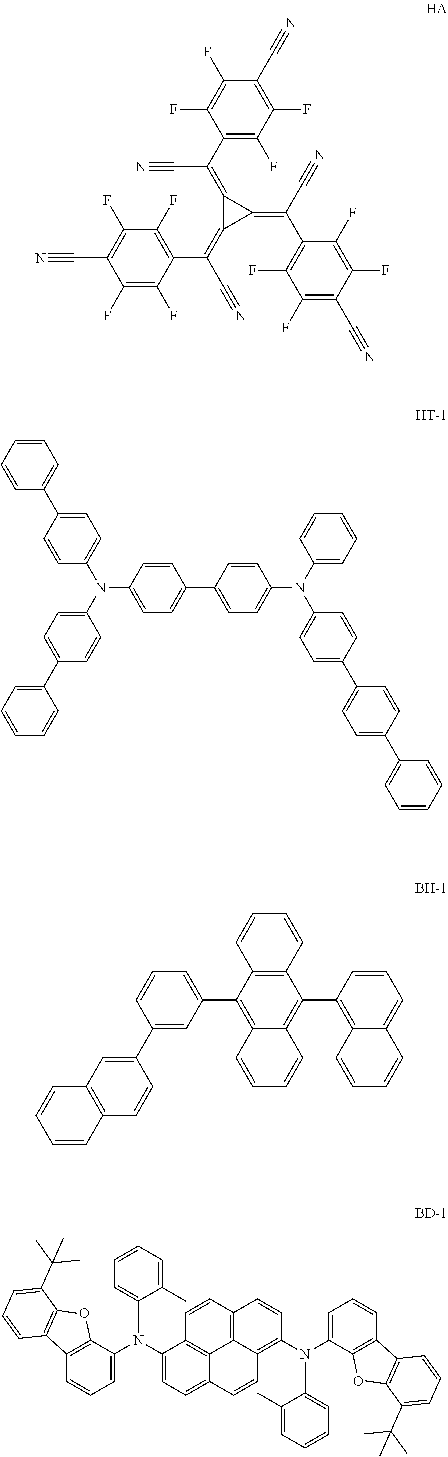

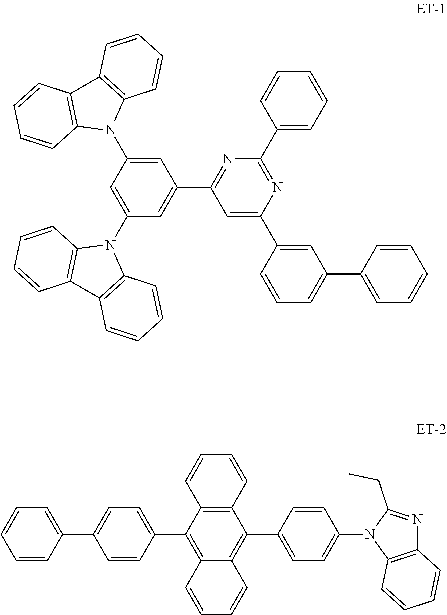 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Production of Organic EL Device

### Example 1

A glass substrate of 25 mm×75 mm×1.1 mm with an ITO transparent electrode (anode) (manufactured by GEOMATEC Co., Ltd.) was ultrasonically cleaned in isopropyl alcohol for 5 minutes and then was subjected to UV ozone cleaning for 30 minutes. The film thickness of the ITO was 130 nm.

The cleaned glass substrate with the transparent electrode was mounted on a substrate holder of a vacuum vapor deposition apparatus, and firstly, Compound HT-1 and Compound HA were vapor co-deposited on the surface having the transparent electrode formed thereon so as to cover the transparent electrode, thus forming a hole injecting layer with a film thickness of 10 nm. The mass ratio of Compound HT-1 and Compound HA (HT-1:HA) was 97:3.

Subsequently, on the hole injecting layer, Compound HT-1 was vapor deposited to form a first hole transporting layer with a film thickness of 80 nm.

Subsequently, on the first hole transporting layer, Compound 1 as Compound HT-2 was vapor deposited to form a second hole transporting layer with a film thickness of 10 nm.

Subsequently, on the second hole transporting layer, Compound BH-1 (host material) and Compound BD-1 (dopant material) were vapor co-deposited to form a light emitting layer with a film thickness of 25 nm. The mass ratio of Compound BH-1 and Compound BD-1 (BH-1:BD-1) was 96:4.

Subsequently, on the light emitting layer, Compound ET-1 was vapor deposited to form a first electron transporting layer with a film thickness of 10 nm.

Subsequently, on the first electron transporting layer, Compound ET-2 was vapor deposited to form a second electron transporting layer with a film thickness of 15 nm.

Subsequently, on the second electron transporting layer, LiF was vapor deposited to form an electron injecting electrode with a film thickness of 1 nm.

Then, on the electron injecting electrode, metal Al was vapor deposited to form a metal cathode with a film thickness of 50 nm.

The layer configuration of the thus obtained organic EL device of Example 1 is shown below.
ITO (130)/HT-1:HA=97:3 (10)/HT-1 (80)/HT-2 (10)/BH-1:BD-1=96:4 (25)/ET-1 (10)/ET-2 (15)/LiF (1)/Al (50)

In the layer configuration, the numerals in parentheses each represent a film thickness (nm) and the ratios are each a mass ratio.

### Example 2

An organic EL devices was produced in the same manner as in Example 1 except for changing the material for the second hole transporting layer from Compound 1 to Compound 2 as shown in Table 1 below.

### Comparative Example 1

An organic EL devices was produced in the same manner as in Example 1 except for changing the material for the second hole transporting layer from Compound 1 to Comparative Compound 1 as shown in Table 1 below.

### Evaluation of Organic EL Devices

### Measurement of Lifetime

For each of the obtained organic EL devices, a voltage was applied on the organic EL device so that the current density was 50 mA/cm², and the 95% lifetime (LT95) was evaluated. The 95% lifetime (LT95) herein means the time period (hr) until the luminance was reduced to 95% of the initial luminance in driving at a constant current.

The results are shown in Table 1.

TABLE 1

| | Material for second | |
|---|---|---|
| | hole transporting | LT95 [hr] |
| | layer (HT-2) | @50 mA/cm² |
| Example 1 | Compound 1 | 76 |
| Example 2 | Compound 2 | 72 |
| Comparative | Comparative | 63 |
| Example 1 | Compound 1 | |

As is apparent from the results shown in Table 1, the monoamines that satisfy the definition of the present invention (Compound 1 of Example 1 and Compound 2 of Example 2) provide significantly improved LT95 values as compared with the monoamine that does not satisfy the definition of the present invention (Comparative compound 1 of Comparative Example 1).

### Other Compounds Used in Production of Organic EL Devices of Examples 3 and 4 and Comparative Examples 2 and 3

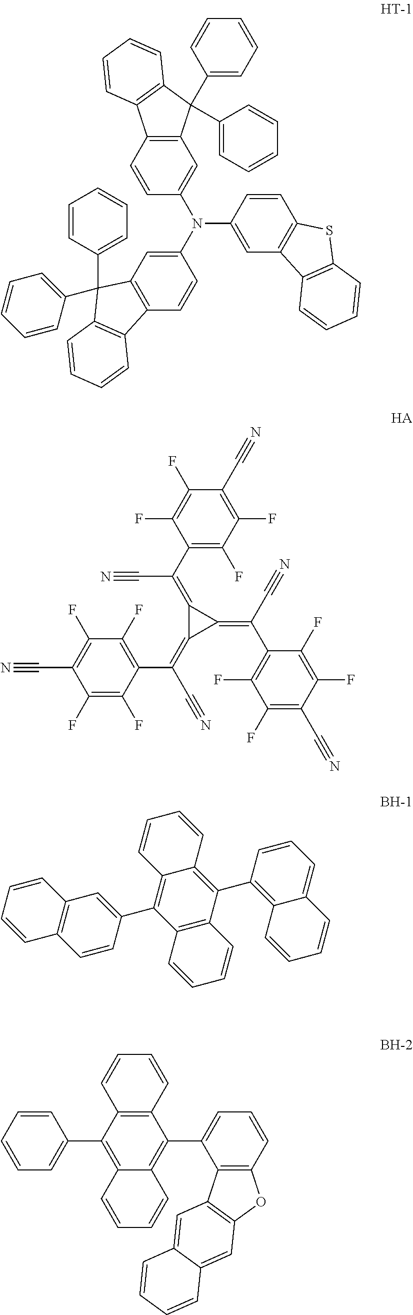

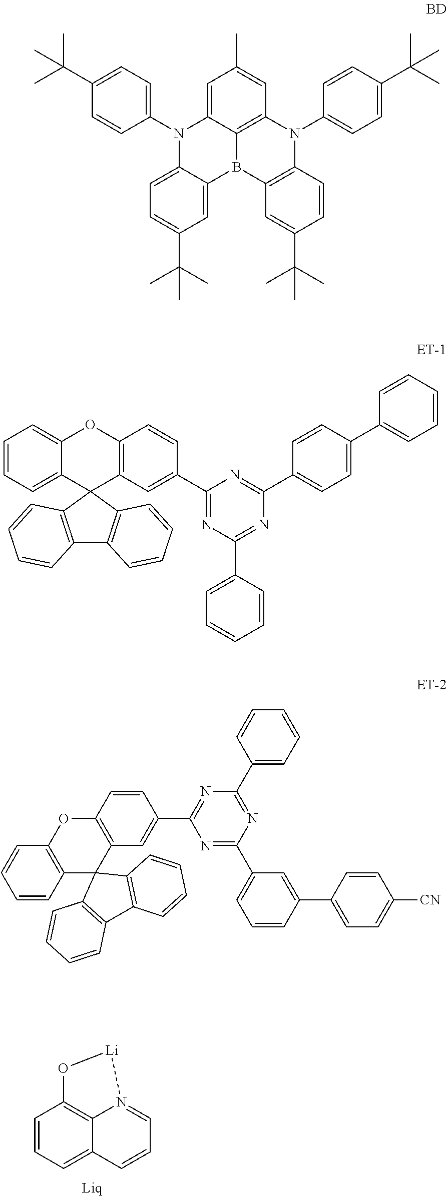

### Production of Organic EL Device

### Example 3

A glass substrate of 25 mm×75 mm×1.1 mm with an ITO transparent electrode (anode) (manufactured by GEOMATEC Co., Ltd.) was ultrasonically cleaned in isopropyl alcohol for 5 minutes and then was subjected to UV ozone cleaning for 30 minutes. The film thickness of the ITO was 130 nm.

The cleaned glass substrate with the transparent electrode was mounted on a substrate holder of a vacuum vapor deposition apparatus, and firstly, Compound HT-1 and Compound HA were vapor co-deposited on the surface having the transparent electrode formed thereon so as to cover the transparent electrode, thus forming a hole injecting layer with a film thickness of 10 nm. The mass ratio of Compound HT-1 and Compound HA (HT-1:HA) was 97:3. Subsequently, on the hole injecting layer, Compound HT-1 was vapor deposited to form a first hole transporting layer with a film thickness of 75 nm.

Subsequently, on the first hole transporting layer, Compound 3 as Compound HT-2 was vapor deposited to form a second hole transporting layer with a film thickness of 7.5 nm.

Subsequently, on the second hole transporting layer, Compound BH (host material) and Compound BD (dopant material) were vapor co-deposited to form a light emitting layer with a film thickness of 20 nm. The mass ratio of Compound BH and Compound BD (BH:BD) was 98:2. The mass ratio of Compound BH-1 and Compound BH-2 (BH-1:BH-2) in Compound BH herein was 60:40.

Subsequently, on the light emitting layer, Compound ET-1 was vapor deposited to form a first electron transporting layer with a film thickness of 6 nm.

Subsequently, on the first electron transporting layer, Compound ET-2 and Compound Liq were vapor co-deposited to form a second electron transporting layer with a film thickness of 27 nm. The mass ratio of Compound ET-2 and Liq (ET-2:Liq) was 67:33.

Subsequently, on the second electron transporting layer, LiF and Yb were vapor co-deposited to form an electron injecting electrode with a film thickness of 1 nm. The mass ratio of LiF and Yb (LiF:Yb) was 50:50.

Then, on the electron injecting electrode, metal Al was vapor deposited to form a metal cathode with a film thickness of 50 nm.

The layer configuration of the thus obtained organic EL device of Example 3 is shown below.
ITO (130)/HT-1:HA=97:3 (10)/HT-1 (75)/HT-2 (7.5)/BH:BD=98:2 (20)/ET-1 (6)/ET-2:Liq=67:33 (27)/LiF:Yb=50:50 (1)/Al (50)

In the layer configuration, the numerals in parentheses each represent a film thickness (nm) and the ratios are each a mass ratio.

### Example 4

An organic EL device was produced in the same manner as in Example 3 except for changing the material for the second hole transporting layer from Compound 3 to Compound 4 as shown in Table 2 below.

### Comparative Example 2

An organic EL device was produced in the same manner as in Example 3 except for changing the material for the second hole transporting layer from Compound 3 to Comparative Compound 2 as shown in Table 2 below.

### Comparative Example 3

An organic EL device was produced in the same manner as in Example 3 except for changing the material for the second hole transporting layer from Compound 3 to Comparative Compound 3 as shown in Table 2 below.

### Evaluation of Organic EL Devices

### Measurement of Lifetime

For each of the obtained organic EL devices, a voltage was applied on the organic EL device so that the current density was 50 mA/cm², and the 95% lifetime (LT95) was evaluated. The 95% lifetime (LT95) herein means the time period (hr) until the luminance was reduced to 95% of the initial luminance in driving at a constant current.

The results are shown in Table 2.

TABLE 2

| | Material for second | |
|---|---|---|
| | hole transporting | LT95 [hr] |
| | layer (HT-2) | @50 mA/cm² |
| Example 3 | Compound 3 | 104 |
| Example 4 | Compound 4 | 110 |
| Comparative | Comparative | 82 |
| Example 2 | Compound 2 | |
| Comparative | Comparative | 85 |
| Example 3 | Compound 3 | |

### <Synthesis of Compound>

### Synthetic Example 1: Synthesis of Intermediate A

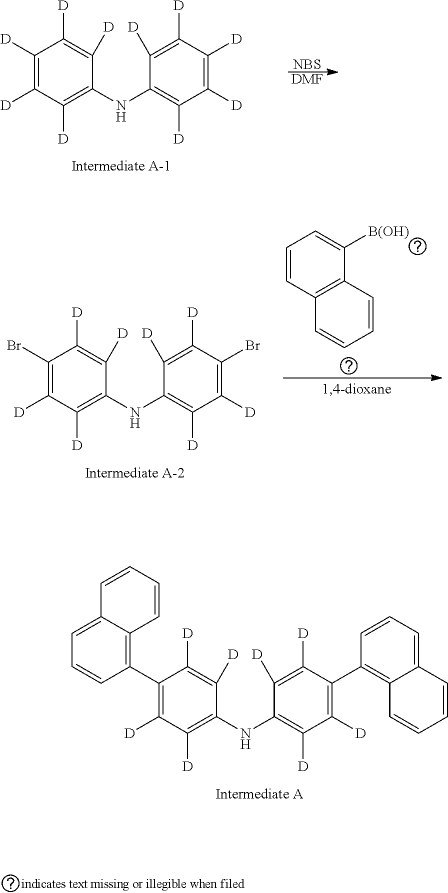

In an argon atmosphere, Intermediate A-1 (2.9 g, 16.18 mmol) and DMF (55 mL) were mixed and N-bromosuccinimide (5.76 g, 32.4 mmol) was added thereto at 0° C. Water and ethyl acetate were added for extraction, and the resulting organic layer was distilled under a reduced pressure to obtain Intermediate A-2. Intermediate A-2 was used in the next reaction without purification.

In an argon atmosphere, Intermediate A-2 (6.41 g, 19.12 mmol), 1-naphthyl boronic acid (8.22 g, 47.8 mmol), bis(di-t-butyl(4-dimethylaminophenyl)phosphine) dichloropalladium(II) (406 mg, 0.574 mmol), and 1,4-dioxane (100 mL) were mixed and an aqueous potassium phosphate solution was added thereto. After the mixture was heated with stirring at 110° C. for 7 hours and was then allowed to cool, the mixture was filtered, followed by purification through column chromatography and recrystallization, to obtain Intermediate A (4.9 g). The yield was 71% (two steps).

### Synthetic Example 2: Synthesis of Intermediate B

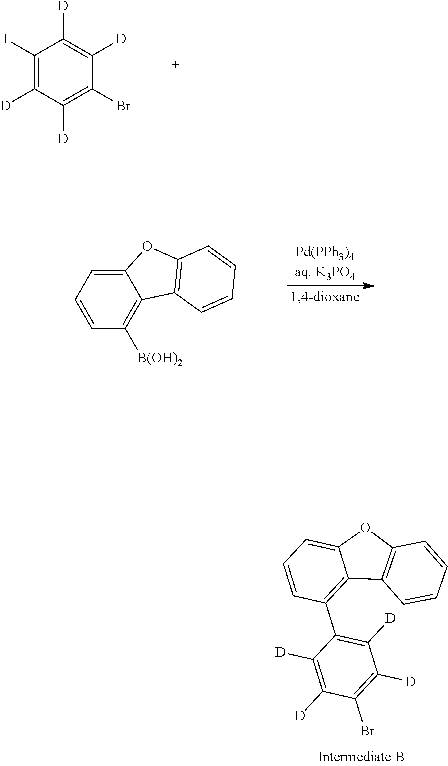 [SMILES: [2H]c1c([2H])c(I)c([2H])c([2H])c1Br ; OB(O)c1cccc2oc3ccccc3c12 ; [2H]c1c([2H])c(-c2cccc3oc4ccccc4c23)c([2H])c([2H])c1Br]

In an argon atmosphere, a mixture of 21.5 g (74.9 mmol) of 3-bromo-6-iodobenzene-1,2,4,5-d4, 25.9 g (75.0 mmol) of dibenzofuran-1-boronic acid, 2.60 g (2.25 mmol) of tetrakis(triphenylphosphine)palladium(0), 93.7 mL of a 2 M aqueous potassium phosphate solution, and 430 mL of 1,4-dioxane was stirred at 80° C. for 9 hours. The reaction solution was cooled to room temperature, and then, water was added thereto, followed by extraction with toluene. The organic layer was concentrated under a reduced pressure, and the resulting residue was purified by silica gel column chromatography to obtain 15.5 g of a white solid. The yield was 63%.

### Synthetic Example 3: Synthesis of Compound 1

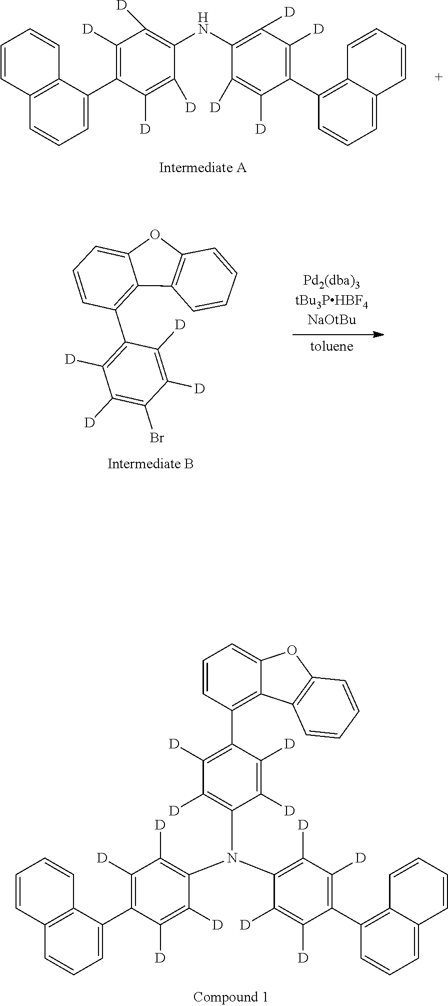 [SMILES: [2H]c1c([2H])c(-c2cccc3ccccc23)c([2H])c([2H])c1Nc1c([2H])c([2H])c(-c2cccc3ccccc23)c([2H])c1[2H] ; [2H]c1c([2H])c(-c2cccc3oc4ccccc4c23)c([2H])c([2H])c1Br ; [2H]c1c([2H])c(N(c2c([2H])c([2H])c(-c3cccc4ccccc34)c([2H])c2[2H])c2c([2H])c([2H])c(-c3cccc4oc5ccccc5c34)c([2H])c2[2H])c([2H])c([2H])c1-c1cccc2ccccc12]

In an argon atmosphere, a mixture of 2.58 g (6.00 mmol) of Intermediate A, 2.06 g (6.30 mmol) of Intermediate B, 0.110 g (0.120 mmol) of tris(dibenzylideneacetone)dipalladium(0), 0.139 g (0.480 mmol) of tri-t-butylphosphonium tetrafluoroborate, 0.807 g (8.40 mmol) of sodium t-butoxide, and 60 mL of toluene was stirred at 100° C. for 7 hours. The reaction solution was cooled to room temperature, and then was concentrated under a reduced pressure. The resulting residue was purified through silica gel column chromatography and recrystallization to obtain 3.48 g of a white solid. The yield was 86%.

As a result of mass spectroscopy, the resulting product was found to be Compound 1 with m/e=676 with respect to the molecular weight of 675.89.

### Synthetic Example 4: Synthesis of Compound 2

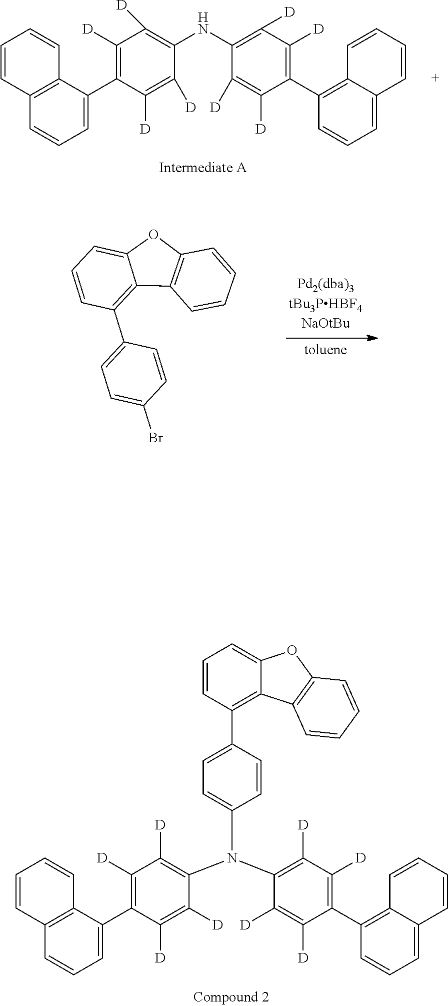 [SMILES: [2H]c1c([2H])c(-c2cccc3ccccc23)c([2H])c([2H])c1Nc1c([2H])c([2H])c(-c2cccc3ccccc23)c([2H])c1[2H] ; Brc1ccc(-c2cccc3oc4ccccc4c23)cc1 ; [2H]c1c([2H])c(N(c2ccc(-c3cccc4oc5ccccc5c34)cc2)c2c([2H])c([2H])c(-c3cccc4ccccc34)c([2H])c2[2H])c([2H])c([2H])c1-c1cccc2ccccc12]

The same operation as in Synthetic Example 1 was performed except for using 1-(4-bromophenyl)dibenzofuran in place of Intermediate B to produce a white solid. The yield was 64%.

As a result of mass spectroscopy, the resulting product was found to be Compound 2 with m/e=672 with respect to the molecular weight of 671.87.

### Synthetic Example 5: Synthesis of Compound 3

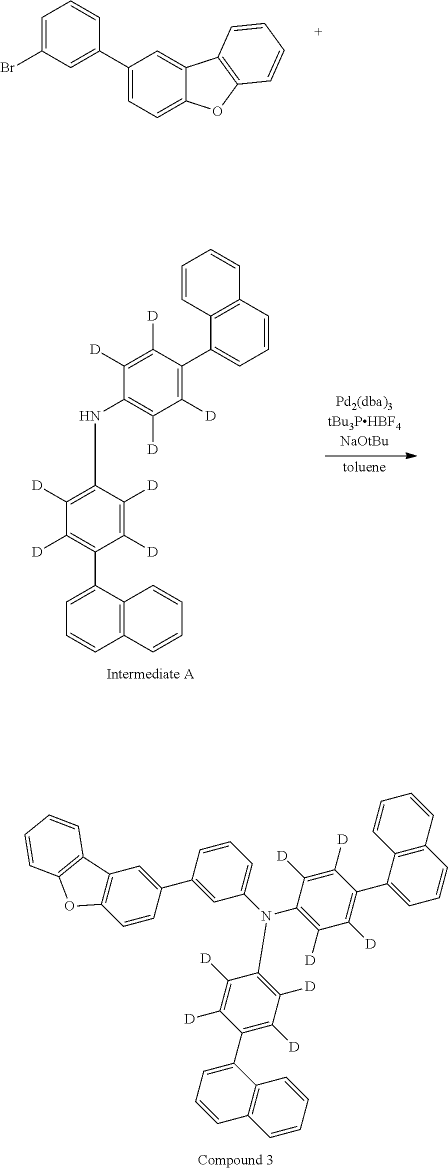 [SMILES: Brc1cccc(-c2ccc3oc4ccccc4c3c2)c1 ; [2H]c1c([2H])c(-c2cccc3ccccc23)c([2H])c([2H])c1Nc1c([2H])c([2H])c(-c2cccc3ccccc23)c([2H])c1[2H] ; [2H]c1c([2H])c(N(c2cccc(-c3ccc4oc5ccccc5c4c3)c2)c2c([2H])c([2H])c(-c3cccc4ccccc34)c([2H])c2[2H])c([2H])c([2H])c1-c1cccc2ccccc12]

The same operation as in Synthetic Example 3 was performed except for using 2-(3-bromophenyl)dibenzofuran in place of Intermediate B to produce a white solid. The yield was 58%.

As a result of mass spectroscopy, the resulting product was found to be Compound 3 with m/e=672 with respect to the molecular weight of 671.87.

### Synthetic Example 6: Synthesis of Compound 4

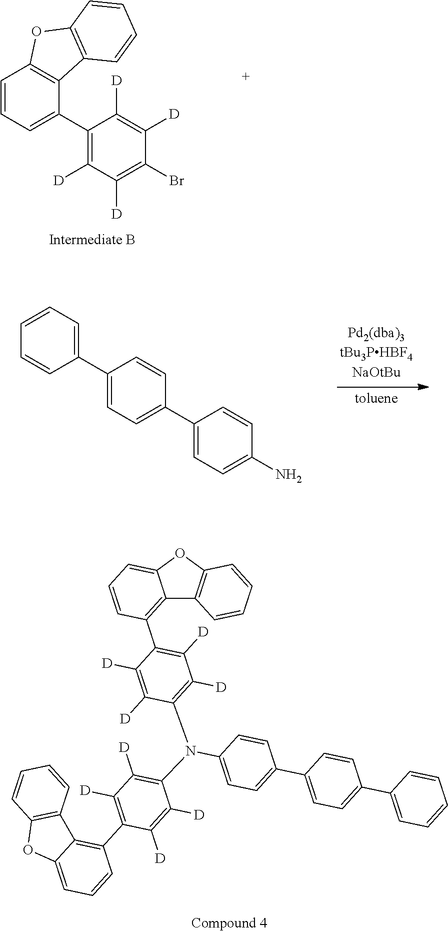 [SMILES: [2H]c1c([2H])c(-c2cccc3oc4ccccc4c23)c([2H])c([2H])c1Br ; Nc1ccc(-c2ccc(-c3ccccc3)cc2)cc1 ; [2H]c1c([2H])c(N(c2ccc(-c3ccc(-c4ccccc4)cc3)cc2)c2c([2H])c([2H])c(-c3cccc4oc5ccccc5c34)c([2H])c2[2H])c([2H])c([2H])c1-c1cccc2oc3ccccc3c12]

The same operation as in Synthetic Example 3 was performed except for using terphenyl-4-amine in place of Intermediate A to produce a white solid. The yield was 46%.

As a result of mass spectroscopy, the resulting product was found to be Compound 4 with m/e=738 with respect to the molecular weight of 737.93.

### REFERENCE SIGNS LIST

1, 11: Organic EL device
2: Substrate
3: Anode
4: Cathode
5: Light emitting layer
6: Hole transporting zone (hole transporting layer)
6a: Hole injecting layer
6b: First hole transporting layer
6c: Second hole transporting layer
6d: Third hole transporting layer
7: Electron transporting zone (electron transporting layer)
7a: First electron transporting layer
7b: Second electron transporting layer
10, 20: Light emitting unit

1: A compound represented by the following formula (1): 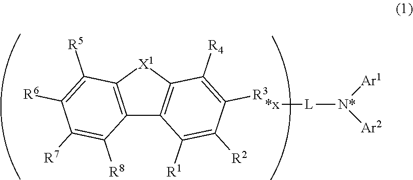
wherein
N* is a central nitrogen atom;
X¹ is an oxygen atom or a sulfur atom;
one selected from R¹ to R³ and R⁶ to R⁸ is a single bond bonded to *x;
R⁴ and R⁵, and R¹ to R³ and R⁶ to R⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R⁴ and R⁵, and R¹ to R³ and R⁶ to R⁸ that are not a single bond each are not bonded to each other and form no ring;
L is an unsubstituted phenylene group;
Ar¹ and Ar² are each independently represented by any of the following formulae (1-a) to (1-f); 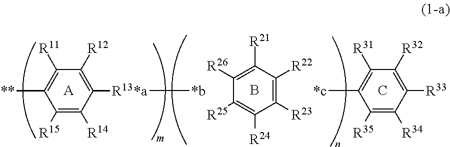
wherein
** represents a binding position to the central nitrogen atom N*;
one selected from R¹¹ to R¹⁵ is a single bond bonded to *a, one selected from R²¹ to R²⁶ is a single bond bonded to *b, and another one selected from R²¹ to R²⁶ is a single bond bonded to *c;
R¹¹ to R¹⁵ that are not a single bond are each independently a hydrogen atom, an unsubstituted alkyl group having 1 to 6 carbon atoms, or an unsubstituted aryl group having 6 to 12 ring carbon atoms;
R²¹ to R²⁶ that are not a single bond are each independently a hydrogen atom, an unsubstituted alkyl group having 1 to 6 carbon atoms, or an unsubstituted aryl group having 6 to 12 ring carbon atoms;
R³¹ to R³⁵ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R¹¹ to R¹⁵ that are not a single bond and of R²¹ to R²⁶ that are not a single bond each are bonded to each other to form a substituted or unsubstituted monocyclic ring having 3 or more and 6 or less ring atoms, or each are not bonded to each other and form no ring;
one or more combinations of combinations each including adjacent two or more of R³¹ to R³⁵ that are not a single bond each are not bonded to each other and form no ring;
m is 0 or 1,
n is 0 or 1, and
m≥n, provided that
when m and n is 0, *c is bonded to the central nitrogen atom N*, and
when m is 1 and n is 0, one selected from R¹¹ to R¹⁵ is a single bond bonded to *c; 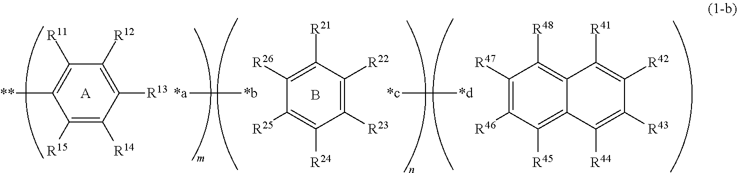
wherein
**, *a, *b, *c, R¹¹ to R¹⁵, R²¹ to R²⁶, m, and n are as defined in the formula (1-a);
one selected from R⁴¹ to R⁴⁸ is a single bond bonded to *d, and R⁴¹ to R⁴⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
when R⁴¹ to R⁴⁸ that are not a single bond are each a hydrogen atom, all the hydrogen atoms are each a protium atom;
one or more combinations of combinations each including adjacent two or more of R⁴¹ to R⁴⁸ that are not a single bond each are not bonded to each other and form no ring; 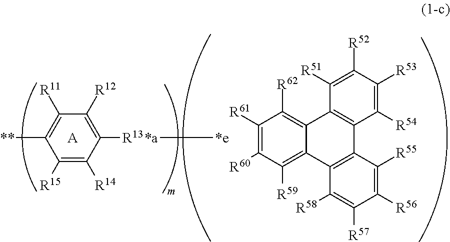
wherein
**, *a, R¹¹ to R¹⁵, and m are as defined in the formula (1-a);
one selected from R⁵¹ to R⁶² is a single bond bonded to *e, and R⁵¹ to R⁶² that are not a single bond are each independently a hydrogen atom or a substituted or unsubstituted alkyl group having 1 to 6 carbon atoms;
one or more combinations of combinations each including adjacent two or more of R⁵¹ to R⁶² that are not a single bond each are not bonded to each other and form no ring;
provided that when m is 0, *a is bonded to the central nitrogen atom N*; 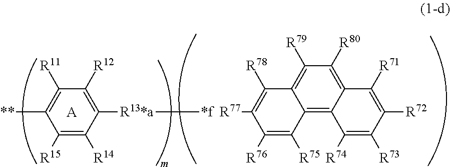
wherein
**, *a, R¹¹ to R¹⁵, and m are as defined in the formula (1-a);
one selected from R⁷¹ to R⁷⁸ is a single bond bonded to *f, and R⁷⁹ and R⁸⁰, and R⁷¹ to R⁷⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R⁷⁹ and R⁸⁰, and R⁷¹ to R⁷⁸ that are not a single bond each are not bonded to each other and form no ring;
provided that when m is 0, *a is bonded to the central nitrogen atom N*; 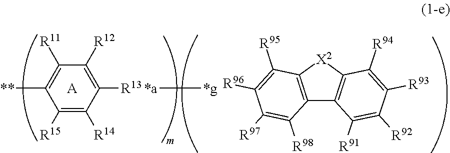
wherein
**, *a, R¹¹ to R¹⁵, and m are as defined in the formula (1-a);
X² is an oxygen atom, a sulfur atom, or CR^{A}R^{B};
R^{A} and R^{B} are each independently a substituted or unsubstituted alkyl group having 1 to 6 carbon atoms, or a substituted or unsubstituted aryl group having 6 to 12 ring carbon atoms;
R^{A} and R^{B} are bonded to each other to form a substituted or unsubstituted monocyclic ring, are bonded to each other to form a substituted or unsubstituted condensed ring, or are not bonded to each other and form no ring;
one selected from R⁹¹ to R⁹⁸ is a single bond bonded to *g, and R⁹¹ to R⁹⁸ that are not a single bond are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
one or more combinations of combinations each including adjacent two or more of R⁹¹ to R⁹⁸ that are not a single bond each are bonded to each other to form a substituted or unsubstituted monocyclic ring having 3 or more and 6 or less ring atoms, or each are not bonded to each other and form no ring;
provided that when m is 0 and X² is an oxygen atom, one selected from R⁹² to R⁹⁷ is a single bond bonded to *g, and *a is bonded to the central nitrogen atom N*; 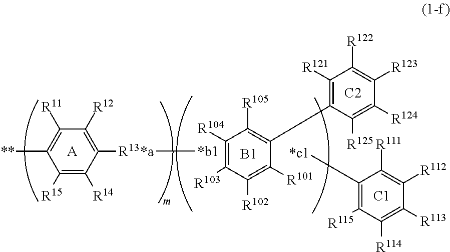
wherein
**, *a, R¹¹ to R¹⁵, and m are as defined in the formula (1-a);
one selected from R¹⁰¹ to R¹⁰⁵ is a single bond bonded to *b1, and another one selected from R¹⁰¹ to R¹⁰⁵ is a single bond bonded to *c1;
R¹⁰¹ to R¹¹⁵ and R¹²¹ to R¹²⁵ are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R¹⁰¹ to R¹⁰⁵ that are not a single bond are each independently a hydrogen atom, an unsubstituted alkyl group having 1 to 6 carbon atoms, or an unsubstituted aryl group having 6 to 12 ring carbon atoms;
one or more combinations of combinations each including adjacent two or more of R¹⁰¹ to R¹⁰⁵ that are not a single bond each are not bonded to each other and form no ring;
one or more combinations of combinations each including adjacent two or more of R¹¹¹ to R¹¹⁵ and R¹²¹ to R¹²⁵ each are not bonded to each other and form no ring;
provided that when m is 0, *a is bonded to the central nitrogen atom N*; and
wherein in the formula (1), at least one of hydrogen atoms contained in L, R¹¹ to R¹⁵ that are not a single bond, R²¹ to R²⁶ that are not a single bond, R⁵¹ to R⁶² that are not a single bond, R⁷¹ to R⁸⁰ that are not a single bond, R⁹¹ to R⁹⁸ that are not a single bond, R^{A} to R^{B}, and R¹⁰¹ to R¹⁰⁵ that are not a single bond is a deuterium atom.

2: The compound according to claim 1, which is represented by one of the following formulae (1-1) to (1-8): 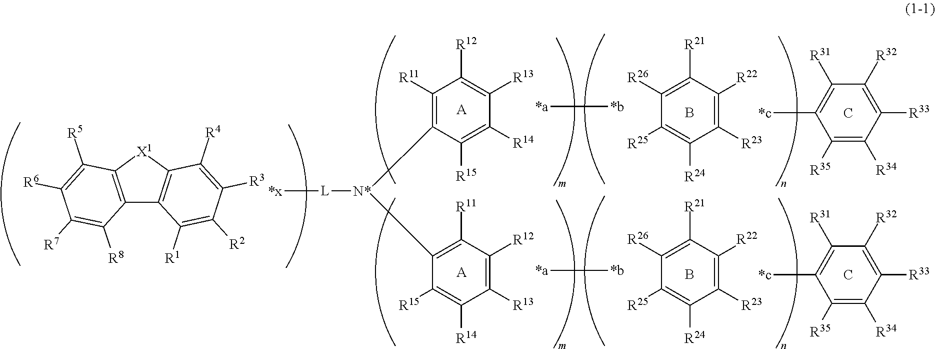
wherein
N*, X¹, R¹ to R⁸, *x, L, *a, *b, *c, R¹¹ to R¹⁵, R²¹ to R²⁶, R³¹ to R³⁵, m, and n are as defined in the formula (1) and the formula (1-a); 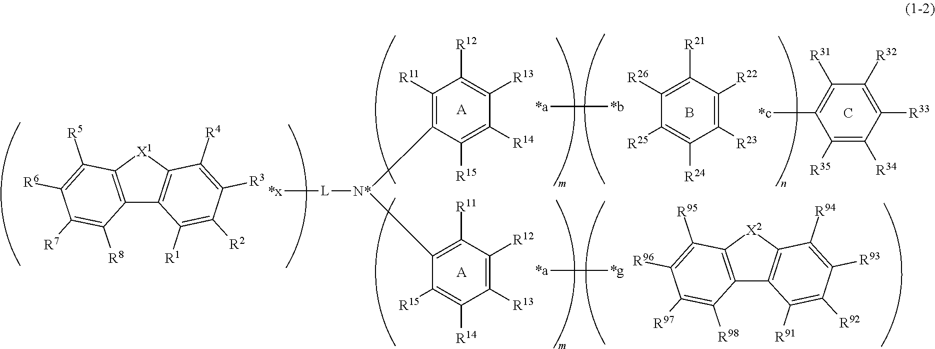
wherein
N*, X¹, R¹ to R⁸, *x, L, *a, *b, *c, *g, R¹¹ to R¹⁵, R²¹ to R²⁶, R³¹ to R³⁵, R⁹¹ to R⁹⁸, X², m, and n are as defined in the formulae (1), (1-a), and (1-e); 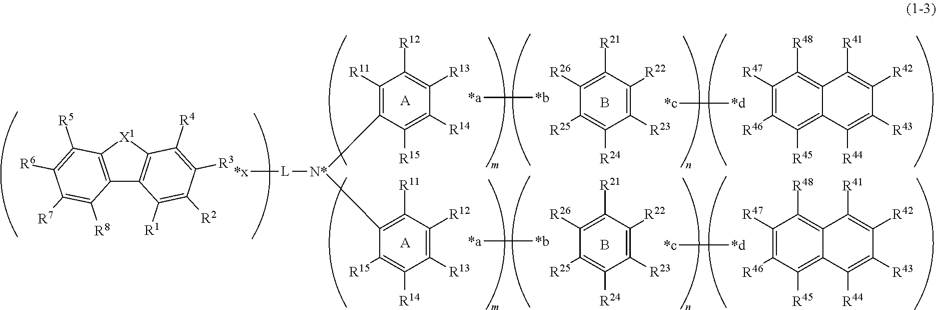
wherein
N*, X¹, R¹ to R⁸, *x, L, *a, *b, *c, *d, R¹¹ to R¹⁵, R²¹ to R²⁶, R⁴¹ to R⁴⁸, m, and n are as defined in the formulae (1) and (1-b); 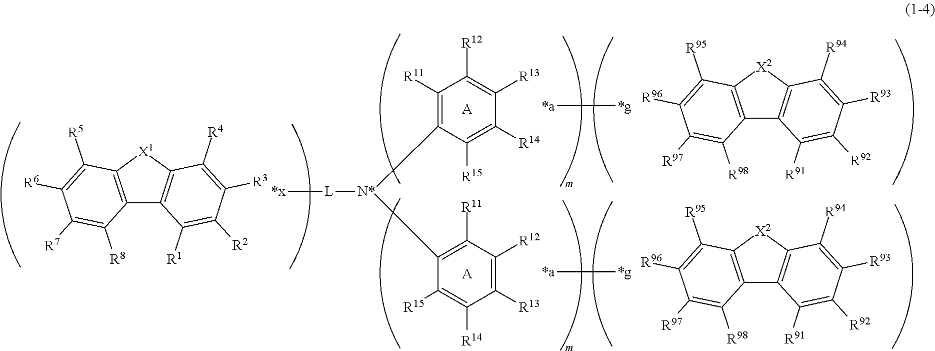
wherein
N*, X¹, R¹ to R⁸, *x, L, *a, *g, R¹¹ to R¹⁵, R⁹¹ to R⁹⁸, X², and m are as defined in the formulae (1) and (1-e); 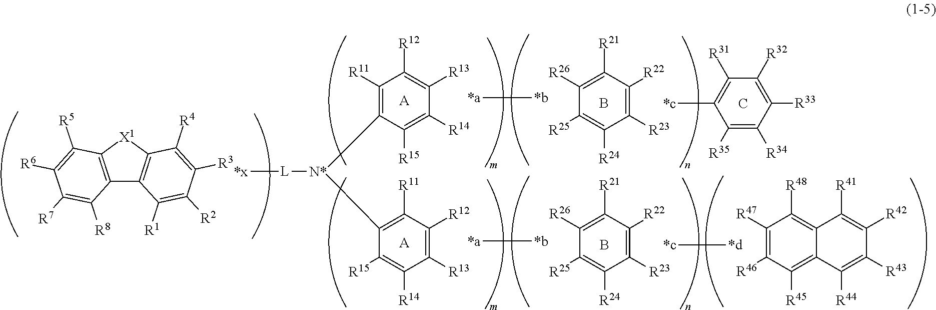
wherein
N*, X¹, R¹ to R, *x L, *a, *b, *c *d, R¹¹ to R¹⁵, R²¹ to R²⁶, R³¹ to R³⁵, R⁴¹ to R⁴⁸, m and n are as defined in the formulae (1), (1-a), and (1-b); 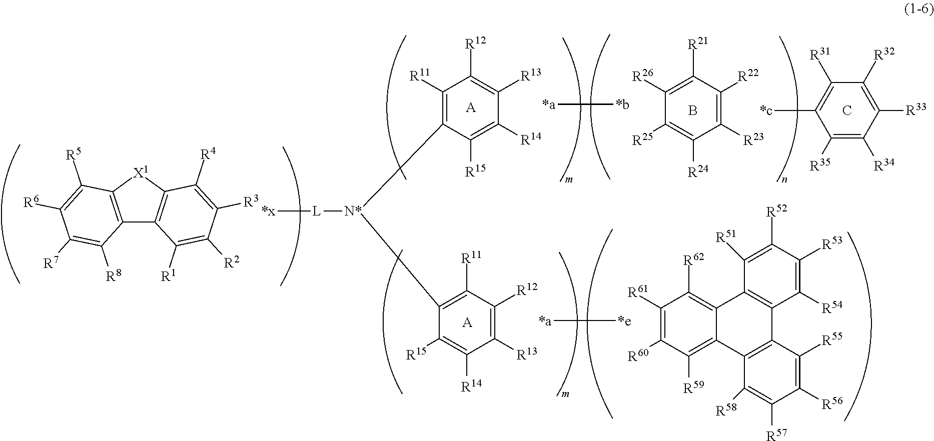
wherein
N*, X¹, R¹ to R, *x L, *a, *b, *c *e, R¹¹ to R¹⁵, R²¹ to R²⁶, R³¹ to R³⁵, R⁵¹ to R⁶², m, and n are as defined in the formulae (1), (1-a), and (1-c). 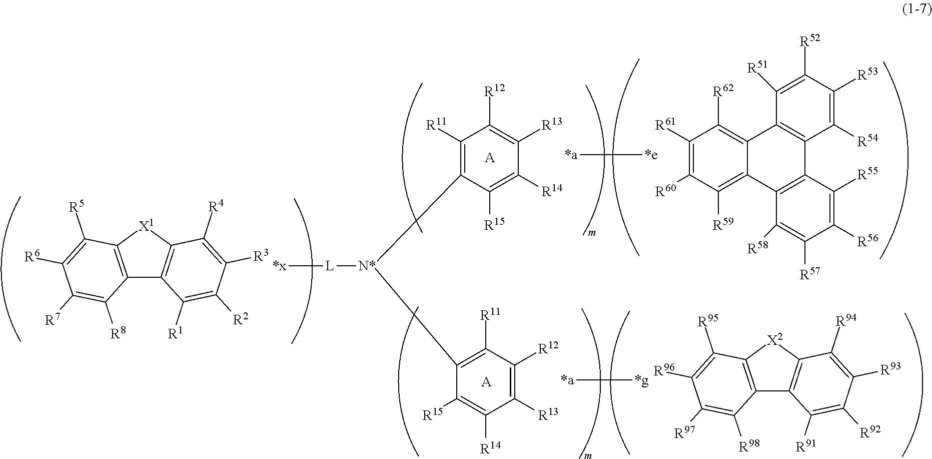
wherein
N*, X¹, R¹ to R⁸, *x, L, *a, *e, *g, R¹¹ to R¹⁵, R⁵¹ to R⁶², R⁹¹ to R⁹⁸, X², and m are as defined in the formulae (1), (1-c), and (1-e); 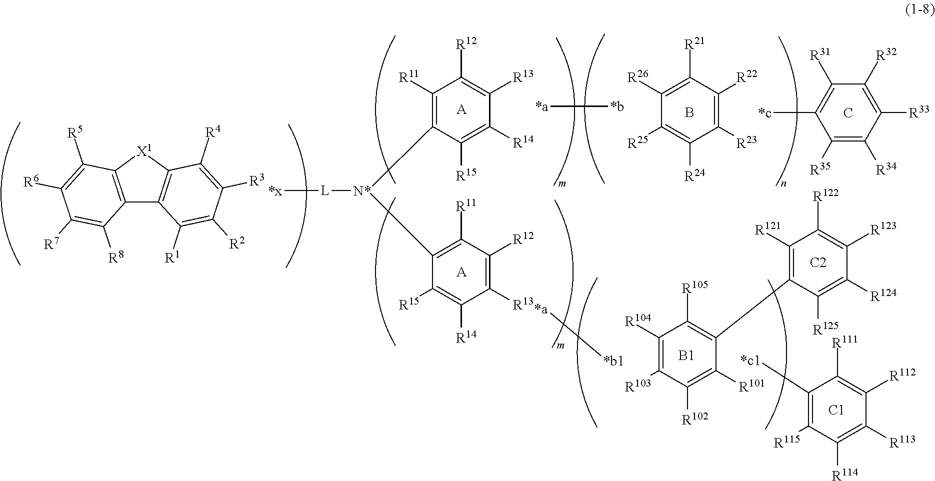
wherein
N*, X¹, R¹ to R⁸, *x, L, *a, *b, *c, *b1, *c1, R¹¹ to R¹⁵, R²¹ to R²⁶, R³¹ to R³⁵, R¹⁰¹ to R¹⁰⁵, R¹¹¹ to R¹⁵, R¹²¹ to R¹²⁵, m, and n are as defined in the formulae (1), (1-a), and (1-f).

3: The compound according to claim 2, which is represented by one of the formulae (1-1) to (1-5).

4: The compound according to claim 1, wherein R⁴ and R⁵, and R¹ to R³ and R⁶ to R⁸ that are not a single bond are each independently a hydrogen atom.

5: The compound according to claim 2, wherein m is 1.

6: The compound according to claim 5, wherein n is 0.

7: The compound according to claim 1, wherein X¹ is an oxygen atom.

8: The compound according to claim 1, wherein at least one of the following (1) to (15) is a deuterium atom:
(1) a hydrogen atom directly bonded to the phenylene group represented by L;
(2) a hydrogen atom represented by R¹¹ to R¹⁵ that are not a single bond, a hydrogen atom represented by R²¹ to R²⁶ that are not a single bond, a hydrogen atom represented by R⁵¹ to R⁶² that are not a single bond, a hydrogen atom represented by R⁷¹ to R⁸⁰ that are not a single bond, a hydrogen atom represented by R⁹¹ to R⁹⁸ that are not a single bond, and a hydrogen atom represented by R¹⁰¹ to R¹⁰⁵ that are not a single bond, which are contained in Ar¹;
(3) a hydrogen atom directly bonded to an alkyl group represented by R¹¹ to R¹⁵ that are not a single bond, a hydrogen atom directly bonded to an alkyl group represented by R²¹ to R²⁶ that are not a single bond, a hydrogen atom directly bonded to an alkyl group represented by R⁵¹ to R⁶² that are not a single bond, a hydrogen atom directly bonded to an alkyl group represented by R⁷¹ to R⁸⁰ that are not a single bond, a hydrogen atom directly bonded to an alkyl group represented by R⁹¹ to R⁹⁸ that are not a single bond, a hydrogen atom directly bonded to an alkyl group represented by R^{A} to R^{B}, and a hydrogen atom directly bonded to an alkyl group represented by R¹⁰¹ to R¹⁰⁵ that are not a single bond, which are contained in Ar¹;
(4) a hydrogen atom directly bonded to a substituent on an alkyl group represented by R¹¹ to R¹⁵ that are not a single bond, a hydrogen atom directly bonded to a substituent on an alkyl group represented by R²¹ to R²⁶ that are not a single bond, a hydrogen atom directly bonded to a substituent on an alkyl group represented by R⁵¹ to R⁶² that are not a single bond, a hydrogen atom directly bonded to a substituent on an alkyl group represented by R⁷¹ to R⁸⁰ that are not a single bond, a hydrogen atom directly bonded to a substituent on an alkyl group represented by R⁹¹ to R⁹⁸ that are not a single bond, a hydrogen atom directly bonded to a substituent on an alkyl group represented by R^{A} to R^{B}, and a hydrogen atom directly bonded to a substituent on an alkyl group represented by R¹⁰¹ to R¹⁰⁵ that are not a single bond, which are contained in Ar¹;
(5) a hydrogen atom directly bonded to an aryl group represented by R¹¹ to R¹⁵ that are not a single bond, a hydrogen atom directly bonded to an aryl group represented by R²¹ to R²⁶ that are not a single bond, a hydrogen atom directly bonded to an aryl group represented by R⁵¹ to R⁶² that are not a single bond, a hydrogen atom directly bonded to an aryl group represented by R⁷¹ to R⁸⁰ that are not a single bond, a hydrogen atom directly bonded to an aryl group represented by R⁹¹ to R⁹⁸ that are not a single bond, a hydrogen atom directly bonded to an aryl group represented by R^{A} to R^{B}, and a hydrogen atom directly bonded to an aryl group represented by R¹⁰¹ to R¹⁰⁵ that are not a single bond, which are contained in Ar¹;
(6) a hydrogen atom directly bonded to a substituent on an aryl group represented by R¹¹ to R¹⁵ that are not a single bond, a hydrogen atom directly bonded to a substituent on an aryl group represented by R²¹ to R²⁶ that are not a single bond, a hydrogen atom directly bonded to a substituent on an aryl group represented by R⁵¹ to R⁶² that are not a single bond, a hydrogen atom directly bonded to a substituent on an aryl group represented by R⁷¹ to R⁸⁰ that are not a single bond, a hydrogen atom directly bonded to a substituent on an aryl group represented by R⁹¹ to R⁹⁸ that are not a single bond, a hydrogen atom directly bonded to a substituent on an aryl group represented by R^{A} to R^{B}, and a hydrogen atom directly bonded to a substituent on an aryl group represented by R¹⁰¹ to R¹⁰⁵ that are not a single bond, which are contained in Ar¹;
(7) a hydrogen atom directly bonded to a heterocyclic group represented by R⁵¹ to R⁶² that are not a single bond, a hydrogen atom directly bonded to a heterocyclic group represented by R⁷¹ to R⁸⁰ that are not a single bond, and a hydrogen atom directly bonded to a heterocyclic group represented by R⁹¹ to R⁹⁸ that are not a single bond, which are contained in Ar¹;
(8) a hydrogen atom directly bonded to a substituent on a heterocyclic group represented by R⁵¹ to R⁶² that are not a single bond, a hydrogen atom directly bonded to a substituent on a heterocyclic group represented by R⁷¹ to R⁸⁰ that are not a single bond, and a hydrogen atom directly bonded to a substituent on a heterocyclic group represented by R⁹¹ to R⁹⁸ that are not a single bond, which are contained in Ar¹;
(9) a hydrogen atom represented by R¹¹ to R¹⁵ that are not a single bond, a hydrogen atom represented by R²¹ to R²⁶ that are not a single bond, a hydrogen atom represented by R⁵¹ to R⁶² that are not a single bond, a hydrogen atom represented by R⁷¹ to R⁸⁰ that are not a single bond, a hydrogen atom represented by R⁹¹ to R⁹⁸ that are not a single bond, and a hydrogen atom represented by R¹⁰¹ to R¹⁰⁵ that are not a single bond, which are contained in Ar²;
(10) a hydrogen atom directly bonded to an alkyl group represented by R¹¹ to R¹⁵ that are not a single bond, a hydrogen atom directly bonded to an alkyl group represented by R²¹ to R²⁶ that are not a single bond, a hydrogen atom directly bonded to an alkyl group represented by R⁵¹ to R⁶² that are not a single bond, a hydrogen atom directly bonded to an alkyl group represented by R⁷¹ to R⁸⁰ that are not a single bond, a hydrogen atom directly bonded to an alkyl group represented by R⁹¹ to R⁹⁸ that are not a single bond, a hydrogen atom directly bonded to an alkyl group represented by R^{A} to R^{B}, and a hydrogen atom directly bonded to an alkyl group represented by R¹⁰¹ to R¹⁰⁵ that are not a single bond, which are contained in Ar²;
(11) a hydrogen atom directly bonded to a substituent on an alkyl group represented by R¹¹ to R¹⁵ that are not a single bond, a hydrogen atom directly bonded to a substituent on an alkyl group represented by R²¹ to R²⁶ that are not a single bond, a hydrogen atom directly bonded to a substituent on an alkyl group represented by R⁵¹ to R⁶² that are not a single bond, a hydrogen atom directly bonded to a substituent on an alkyl group represented by R⁷¹ to R⁸⁰ that are not a single bond, a hydrogen atom directly bonded to a substituent on an alkyl group represented by R⁹¹ to R⁹⁸ that are not a single bond, a hydrogen atom directly bonded to a substituent on an alkyl group represented by R^{A} to R^{B}, and a hydrogen atom directly bonded to a substituent on an alkyl group represented by R¹⁰¹ to R¹⁰⁵ that are not a single bond, which are contained in Ar²;
(12) a hydrogen atom directly bonded to an aryl group represented by R¹¹ to R¹⁵ that are not a single bond, a hydrogen atom directly bonded to an aryl group represented by R²¹ to R²⁶ that are not a single bond, a hydrogen atom directly bonded to an aryl group represented by R⁵¹ to R⁶² that are not a single bond, a hydrogen atom directly bonded to an aryl group represented by R⁷¹ to R⁸⁰ that are not a single bond, a hydrogen atom directly bonded to an aryl group represented by R⁹¹ to R⁹⁸ that are not a single bond, a hydrogen atom directly bonded to an aryl group represented by R^{A} to R^{B}, and a hydrogen atom directly bonded to an aryl group represented by R¹⁰¹ to R¹⁰⁵ that are not a single bond, which are contained in Ar²;
(13) a hydrogen atom directly bonded to a substituent on an aryl group represented by R¹¹ to R¹⁵ that are not a single bond, a hydrogen atom directly bonded to a substituent on an aryl group represented by R²¹ to R²⁶ that are not a single bond, a hydrogen atom directly bonded to a substituent on an aryl group represented by R⁵¹ to R⁶² that are not a single bond, a hydrogen atom directly bonded to a substituent on an aryl group represented by R⁷¹ to R⁸⁰ that are not a single bond, a hydrogen atom directly bonded to a substituent on an aryl group represented by R⁹¹ to R⁹⁸ that are not a single bond, a hydrogen atom directly bonded to a substituent on an aryl group represented by R^{A} to R^{B}, and a hydrogen atom directly bonded to a substituent on an aryl group represented by R¹⁰¹ to R¹⁰⁵ that are not a single bond, which are contained in Ar²;
(14) a hydrogen atom directly bonded to a heterocyclic group represented by R⁵¹ to R⁶² that are not a single bond, a hydrogen atom directly bonded to a heterocyclic group represented by R⁷¹ to R⁸⁰ that are not a single bond, and a hydrogen atom directly bonded to a heterocyclic group represented by R⁹¹ to R⁹⁸ that are not a single bond, which are contained in Ar²;
(15) a hydrogen atom directly bonded to a substituent on a heterocyclic group represented by R⁵¹ to R⁶² that are not a single bond, a hydrogen atom directly bonded to a substituent on a heterocyclic group represented by R⁷¹ to R⁸⁰ that are not a single bond, and a hydrogen atom directly bonded to a substituent on a heterocyclic group represented by R⁹¹ to R⁹⁸ that are not a single bond, which are contained in Ar².

9: The compound according to claim 5, wherein all hydrogen atoms represented by R¹¹ to R¹⁵ that are not a single bond are each a deuterium atom.

10: The compound according tom claim 1, wherein all hydrogen atoms directly bonded to the phenylene group represented by L are each a deuterium atom.

11: The compound according to claim 1, wherein the compound represented by the formula (1) has a deuteration ratio of 10% or more.

12: A material for organic electroluminescent devices, the material comprising the compound according to claim 1.

13: The material for organic electroluminescent devices according to claim 12, wherein the material further comprises a protium form of the compound, the protium form being a compound in which all hydrogen atoms are each a protium atom.

14: The material for organic electroluminescent devices according to claim 12, wherein the material is a material for a hole transporting layer.

15: An organic electroluminescent device comprising a cathode, an anode, and organic layers between the cathode and the anode, the organic layers including a light emitting layer, at least one layer of the organic layers containing the compound according to claim 1.

16: The organic electroluminescent device according to claim 15, wherein the organic layers comprise a hole transporting zone between the anode and the light emitting layer, the hole transporting zone containing the compound.

17: The organic electroluminescent device according to claim 16, wherein the hole transporting zone comprises a first hole transporting layer on the side of the anode and a second hole transporting layer on the side of the cathode,
at least one of the first hole transporting layer and the second hole transporting layer containing the compound.

18: The organic electroluminescent device according to claim 15, wherein the light emitting layer comprises a fluorescent dopant material.

19: The organic electroluminescent device according to claim 15, wherein the light emitting layer comprises a phosphorescent dopant material.

20: An electronic appliance comprising the organic electroluminescent device according to claim 15.
